(19) United States
(12) Patent Application Publication
(10) Pub. No.: US 2025/0188098 A1
(43) Pub. Date: Jun. 12, 2025

(54) CARBORANE COMPOUNDS AND METHODS OF USE THEREOF

(71) Applicant: Ohio State Innovation Foundation, Columbus, OH (US)

(72) Inventors: Christopher Charles COSS, Upper Arlington, OH (US); Chad BENNETT, Columbus, OH (US); Tyler WILSON, Columbus, OH (US); Sandip VIBHUTE, Columbus, OH (US); Hanna RADOMSKA, Columbus, OH (US); Samuel KULP, Columbus, OH (US)

(21) Appl. No.: 18/971,255

(22) Filed: Dec. 6, 2024

Related U.S. Application Data
(60) Provisional application No. 63/607,837, filed on Dec. 8, 2023.

(51) Int. Cl. C07F 5/02; A61K 31/69; A61K 49/00

(52) U.S. Cl. CPC C07F 5/027; A61K 31/69; A61K 49/0008

(57) ABSTRACT

Disclosed herein are compounds comprising dicarba-closo-dodecaborane. The compounds can be, for example, estrogen receptor beta (ERβ) agonists. In some examples, the compounds can be selective ERβ agonists. Also provided herein are methods of treating, preventing, or ameliorating cancer in a subject, suppressing tumor growth in a subject, treating an inflammatory disease in a subject, treating a neurodegenerative disease in a subject, treating a psychotropic disorder in a subject, or a combination thereof, by administering to a subject a therapeutically effective amount of one or more of the compounds or compositions described herein, or a pharmaceutically acceptable salt thereof.

### CROSS-REFERENCE TO RELATED APPLICATIONS

This application claims the benefit of priority to U.S. Provisional Application No. 63/607,837, filed Dec. 8, 2023, which is hereby incorporated herein by reference in its entirety.

### BACKGROUND

Estrogen can influence the growth, differentiation, and functioning of many tissues. For example, estrogens play an important role in the female and male reproductive systems, and also in bone maintenance, the central nervous system, and the cardiovascular system. Because of their beneficial actions in non-reproductive tissues, such as bone, brain, and urogenital tract, estrogens would be ideal drugs if they did not have serious adverse effects, such as increasing the risk of breast cancer, endometrial cancer, thromboembolisms, and strokes.

The physiological functions of estrogenic compounds are modulated largely by the estrogen receptor subtypes alpha (ERα) and beta (ERβ). The activity of the two ER subtypes is controlled by the binding of the endogenous hormone 17β-estradiol or of synthetic nonhormonal compounds to the ligand-binding domain.

In humans, both receptor subtypes are expressed in many cells and tissues, and they can control physiological functions in various organ systems, such as reproductive, skeletal, cardiovascular, and central nervous systems, as well as in specific tissues (such as breast and subcompartments of prostate and ovary). ERα is present mainly in mammary glands, uterus, ovary (thecal cells, bone, male reproductive organs (testes and epididumis), prostate (stroma), liver, and adipose tissue. By contrast, ERβ is found mainly in the prostate (epithelium), bladder, ovary (granulosa cells), colon, adipose tissue, and immune system. Both subtypes are markedly expressed in the cardiovascular and central nervous systems, There are some common physiological roles for both estrogen receptor subtypes, such as in the development and function of the ovaries, and in the protection of the cardiovascular system. The alpha subtypes has a more prominent roles on the mammary gland and uterus, as well as on the preservation of skeletal homeostasis and the regulation of metabolism, The beta subtype seems to have a more pronounced effect on the central nervous and immune systems, and it general counteracts the ERα-promoted cell hyperproliferation in tissues such as breast and uterus.

Compounds that either induce or inhibit cellular estrogen responses have potential value as biochemical tools and candidates for drug development. Most estrogen receptor modulators are non-selective for the ER subtypes, but is has been proposed that compounds with ER subtype selectivity would be useful. However, the development of compounds possessing ER subtype specificity still constitutes a major challenge, as the ligand binding domains of the two subtypes are very similar in structure and amino acid sequence.

### SUMMARY

Disclosed herein are compounds comprising dicarba-closo-dodecaborane. The compounds can be, for example, estrogen receptor beta (ERβ) agonists. In some examples, the compounds can be selective ERβ agonists. Also provided herein are methods of treating, preventing, or ameliorating cancer in a subject, suppressing tumor growth in a subject, treating an inflammatory disease in a subject, treating a neurodegenerative disease in a subject, treating a psychotropic disorder in a subject, or a combination thereof, by administering to a subject a therapeutically effective amount of one or more of the compounds or compositions described herein, or a pharmaceutically acceptable salt thereof.

For example, disclosed herein are compounds defined by Formula I, or a pharmaceutically acceptable salt thereof:

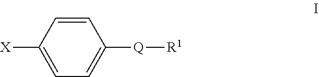

wherein
Q is a substituted or unsubstituted dicarba-closo-dodecaborane cluster, and

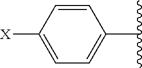

and R¹ are attached to Q in a meta configuration;
X is OH, NHR², SH, or S(O)(O)NHR²;
R¹ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)NR³R⁴, or NR³R⁴;
R² is H, OH, halogen, or substituted or unsubstituted C₁-C₄ alkyl;
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl.

In some examples, Q is

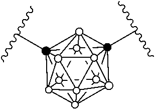

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples, the compound is defined by Formula II, or a pharmaceutically acceptable salt thereof:

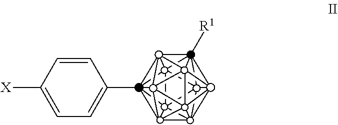

wherein
● is a carbon atom;
◯ is B—H, B-halogen, B-alkyl, B—OH, or B—NH₂;
X is OH, NHR², SH, or S(O)(O)NHR²;
R¹ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)NR³R⁴, or NR³R⁴;
R² is H, OH, halogen, or substituted or unsubstituted C₁-C₄ alkyl;
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl.

In some examples, X is OH.

In some examples, when X is OH, R¹ is not (CH₂)₅CH(CH₃)₂ or NH₂.

In some examples, R¹ is substituted or unsubstituted C₁-C₂₀ alkyl.

In some examples, X is OH and R¹ is substituted or unsubstituted C₁-C₂₀ alkyl.

In some examples, R¹ is C₁-C₂₀ alkyl, optionally substituted with OH, sulfonyl, thiol, or a combination thereof.

In some examples, R¹ is an unsubstituted C₁-C₂₀ alkyl.

In some examples, R¹ is a substituted or unsubstituted C₁-C₁₀ alkyl.

In some examples, R¹ is C₁-C₁₀ alkyl, optionally substituted with OH, sulfonyl, thiol, or a combination thereof.

In some examples, R¹ is an unsubstituted C₁-C₁₀ alkyl, such as an unsubstituted C₁-C₆ alkyl.

In some examples, R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)NR³R⁴, or NR³R⁴.

In some examples, R¹ is substituted or unsubstituted C₄-C₂₀ alkyl.

In some examples, X is OH and R¹ is substituted or unsubstituted C₄-C₂₀ alkyl.

In some examples, R¹ is C₄-C₂₀ alkyl, optionally substituted with OH, sulfonyl, thiol, or a combination thereof.

In some examples, R¹ is an unsubstituted C₄-C₂₀ alkyl.

In some examples, R¹ is a substituted or unsubstituted C₄-C₁₀ alkyl.

In some examples, R¹ is C₄-C₁₀ alkyl, optionally substituted with OH, sulfonyl, thiol, or a combination thereof.

In some examples, R¹ is an unsubstituted C₄-C₁₀ alkyl, such as an unsubstituted C₄-C₆ alkyl.

In some examples, R¹ is

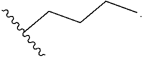 [SMILES: *CCCC]

In some examples, ● is a carbon atom and ◯ is B—H.

Also disclosed herein are compounds defined by Formula XI, or a pharmaceutically acceptable salt thereof

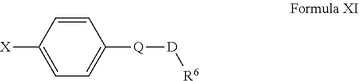

wherein
Q is a substituted or unsubstituted dicarba-closo-dodecaborane cluster, and

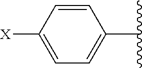

and D are attached to Q in a meta configuration;
D is —S—, —S(O)—, —S(O)(O)—, —S(O)(NH)—, —P(O)(OH)O—, —P(O)(OH)NH—, or —O—;
X is OH, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, or substituted or unsubstituted C₄-C₂₀ alkylheterocycloalkyl; and
R² is H, OH, halogen, or substituted or unsubstituted C₁-C₄ alkyl.

In some examples, Q is

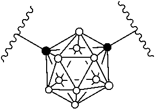

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples, the compound is defined by Formula XIA, or a pharmaceutically acceptable salt thereof:

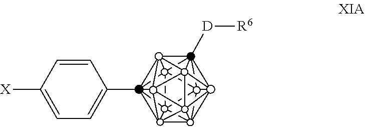

wherein
● is a carbon atom or a boron atom; and
◯ is C—H, C-halogen, C-alkyl, C—OH, C—NH₂, B—H, B-halogen, B-alkyl, B—OH, or B—NH₂
D is —S—, —S(O)—, —S(O)(O)—, —S(O)(NH)—, —P(O)(OH)O—, —P(O)(OH)NH—, or —O—;
X is OH, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, or substituted or unsubstituted C₄-C₂₀ alkylheterocycloalkyl; and
R² is H, OH, halogen, or substituted or unsubstituted C₁-C₄ alkyl.

In some examples, X is OH.

In some examples, D is —S—, or —S(O)(O)—.

In some examples, R⁶ is substituted or unsubstituted C₁-C₂₀ alkyl.

In some examples, R⁶ is C₁-C₂₀ alkyl optionally substituted with OH.

In some examples, R⁶ is substituted or unsubstituted C₁-C₆ alkyl.

In some examples, R⁶ is substituted or unsubstituted C₁-C₃ alkyl.

In some examples, R⁶ is unsubstituted C₁-C₃ alkyl.

In some examples, R⁶ is C₁-C₃ alkyl substituted with OH.

In some examples, R⁶ is selected from the group consisting of: OH

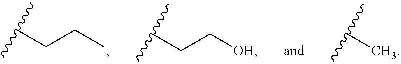

In some examples, R⁶ is selected from the group consisting of:

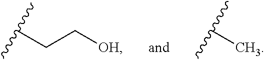

In some examples, R⁶ is

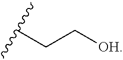 [SMILES: *CCO]

In some examples, D-R⁶ is selected from the group consisting of:

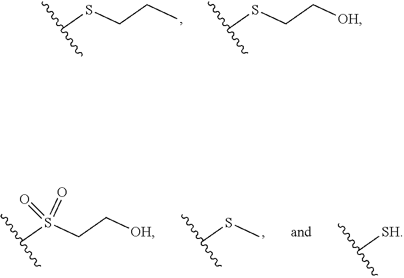

In some examples, D-R⁶ is selected from the group consisting of:

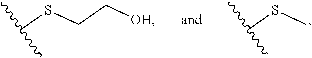

In some examples, D-R⁶ is

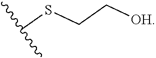 [SMILES: *SCCO]

In some examples, ● is a carbon atom and ◯ is B—H.

Also disclosed herein are compounds comprising:

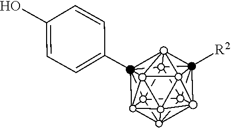

wherein ● is a carbon atom; ◯ is B—H; and R² is selected from the group consisting of:

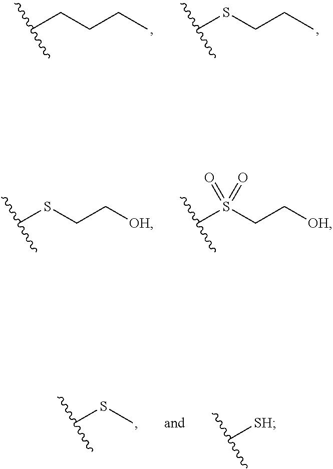

and pharmaceutically acceptable salts thereof.

In some examples, the compound is selected from the group consisting of:

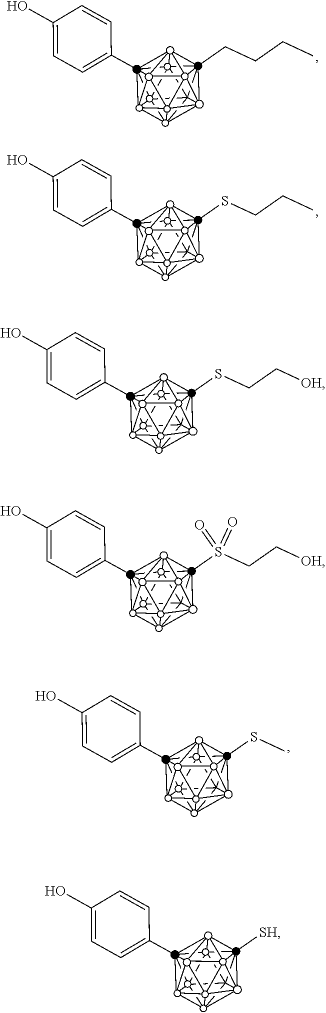

and pharmaceutically acceptable salts thereof, where ● is a carbon atom, and ◯ is B—H.

In some examples, the compound is selected from the group consisting of:

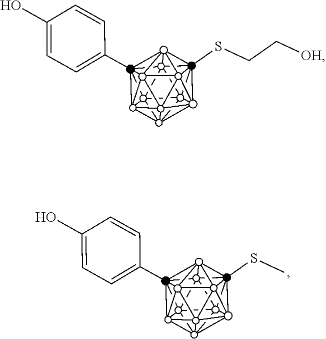

and pharmaceutically acceptable salts thereof, where ● is a carbon atom, and ◯ is B—H.

In some examples, the compound is:

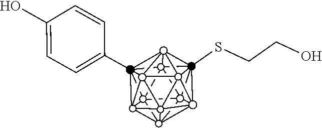

or a pharmaceutically acceptable salt thereof, where ● is a carbon atom, and ◯ is B—H.

In some examples, the compound is a selective ERβ agonist.

In some examples, the compound is a selective ERβ agonist in both a human model and a mouse model.

In some examples, the compound has an EC₅₀ of 800 nM or less at estrogen receptor beta (ERβ).

In some examples, the compound has an EC₅₀ of 200 nM or less at estrogen receptor beta (ERβ).

In some examples, the compound has an EC₅₀ of 200 nM or less at estrogen receptor beta (ERβ) in both a human model and a mouse model.

In some examples, the compound has an ERβ-to-ERα agonist ratio of 8 or more.

In some examples, the compound has an ERβ-to-ERα agonist ratio of 8 or more in both a human model and a mouse model.

In some examples, the carborane cluster includes a heteroatom.

In some examples, the carborane cluster includes an isotopically labeled atom. In some examples, the isotopically labeled atom includes ¹⁰B. In some examples, the isotopically labeled atom includes a radiohalogen bound to the carborane cluster.

Also disclosed herein are pharmaceutical compositions comprising any of the compounds disclosed herein and a pharmaceutically acceptable excipient.

Also disclosed herein are methods of evaluating the clinical efficacy of an ERβ agonist in a human patient, the method comprising administering the ERβ agonist to a non-human preclinical species model; wherein the ERβ agonist has an ERβ-to-ERα ratio of 8 or more in both a human model and the non-human preclinical species model. In some examples, the ERβ agonist comprises any of the compounds disclosed herein. In some examples, the preclinical species model comprises a mouse model. In some examples, the method is in vitro or in vivo.

Also disclosed herein are methods of treating cancer in a subject comprising administering to the subject a therapeutically effective amount of any of the compounds or compositions disclosed herein. In some examples, the cancer is selected from the group consisting of breast cancer, colorectal cancer, endometrial cancer, ovarian cancer, and prostate cancer. In some examples, the method further comprises co-administering an anticancer agent to the subject.

Also disclosed herein are methods of suppressing tumor growth in a subject, comprising contacting at least a portion of the tumor with a therapeutically effective amount of any of the compounds or compositions disclosed herein.

Also disclosed herein are methods of treating an inflammatory disease in a subject comprising administering to the subject a therapeutically effective amount of any of the compounds or compositions disclosed herein. In some examples, the inflammatory disease is selected from the group consisting of arthritis and inflammatory bowel disease. In some examples, the method further comprises co-administering an anti-inflammatory agent to the subject.

Also disclosed herein are methods of treating a neurodegenerative disease in a subject comprising administering to the subject a therapeutically effective amount of any of the compounds or compositions disclosed herein.

Also disclosed herein are methods of treating a psychotropic disorder in a subject comprising administering to the subject a therapeutically effective amount of any of the compounds or compositions disclosed herein.

Also disclosed herein are methods of imaging a cell or a population of cells expressing ERβ within or about a subject, the methods comprising: administering to the subject an amount of any of the compounds or compositions disclosed herein; and detecting said compound or composition. In some examples, the cell or population of cells is indicative of cancer, an inflammatory disease, a neurodegenerative disease, a psychotropic disorder, or a combination thereof. In some examples, the cancer is selected from the group consisting of breast cancer, colorectal cancer, and prostate cancer. In some examples, the inflammatory disease is selected from the group consisting of arthritis and inflammatory bowel disease.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. Chemical structure of OSU-ERβ-12, para- and meta-series of carborane analogs FIG. 2A. Human ERα and ERβ in vitro transcriptional activation by OSU-ERβ-12, representing triplicate experiments performed in three separate passages of cells. Data for the remaining para-substituted compounds are found in FIG. 36A-FIG. 36E. EC₅₀ are reported in nM and Error bars represent mean±SEM.

FIG. 2B. Human ERα and ERβ in vitro transcriptional activation by Compound 10, representing triplicate experiments performed in three separate passages of cells. Data for the remaining meta-substituted compounds are found in FIG. 37A-FIG. 37E. EC₅₀ are reported in nM and Error bars represent mean±SEM.

FIG. 2C. Concentration time profile for OSU-ERβ-12. Black and gray lines represent plasma concentration after oral and intravenous administration, respectively. Pharmacokinetic data for the remaining para-substituted compounds are found in FIG. 38A-FIG. 38F. Error bars represent mean±STD.

FIG. 2D. Concentration time profile for compound 10. Black and gray lines represent plasma concentration after oral and intravenous administration, respectively. Pharmacokinetic data for the remaining meta-substituted compounds are found in FIG. 39A-FIG. 39F. Error bars represent mean±STD.

FIG. 2E. Uterotrophic stimulation by OSU-ERβ-12 in estrogen-naïve female mice (All data [left] and curve fits [right]). Separate vehicle controls were performed for the sub-cutaneous and oral vehicles in the OSU-ERβ-12 experiment. Data are presented as mean±SD and were analyzed by One-way ANOVA in comparison with vehicle control. ****, ***, **, * correspond to p<0.0001, 0.001, 0.01 and 0.05, respectively. Uterotrophic data for the remaining para-substituted compounds can be found in FIG. 40A-FIG. 40E.

FIG. 2F. Uterotrophic stimulation by compound 10 in estrogen-naïve female mice (All data [left] and curve fits [right]). Data are presented as mean±SD and were analyzed by One-way ANOVA in comparison with vehicle control. ****, ***, **, * correspond to p<0.0001, 0.001, 0.01 and 0.05, respectively. Uterotrophic data for the remaining meta-substituted compounds can be found in FIG. 41A-FIG. 41E.

FIG. 2G. Mouse ERα and ERβ in vitro transcriptional activation by OSU-ERβ-12, representing triplicate experiments performed in three separate passages of cells. Data for the remaining para-substituted compounds are found in FIG. 42A-FIG. 42E. EC₅₀ are reported in nM and Error bars represent mean±SEM.

FIG. 2H. Mouse ERα and ERβ in vitro transcriptional activation by Compound 10, representing triplicate experiments performed in three separate passages of cells. Data for the remaining meta-substituted compounds are found in FIG. 43A-FIG. 43E. EC₅₀ are reported in nM and Error bars represent mean±SEM.

FIG. 3A. Uterotrophic stimulation by OSU-ERβ-12 and para-substituted analogs in estrogen-naïve female mice. Analogs were tested following 4 consecutive oral daily doses at dose levels ranging from (3-300) mg/kg. Regression lines are presented as a percentage of uterotrophic stimulation by estradiol (E2). The results show that Compound 5 was the most potent analog within the para-series when compared with E2, while OSU-ERβ-12 was the least potent. In the case of Compound 1 and Compound 4 it was not possible to fit a dose-response curve because they demonstrated a 100% uterotrophic effect at the lowest doses tested.

FIG. 3B. Uterotrophic stimulation by OSU-ERβ-12 and meta-substituted analogs in estrogen-naïve female mice. Analogs were tested following 4 consecutive oral daily doses at dose levels ranging from (3-300) mg/kg. Regression lines are presented as a percentage of uterotrophic stimulation by estradiol (E2). The results show that Compound 8 was the least potent analog within the meta-series to induce a uterotrophic effect when compared with E2, while other analogs tested had similar potencies as OSU-ERβ-12 in inducing a uterotrophic effect. Compound 9 did not display any uterotrophic activity even at the highest dose tested.

FIG. 4. Scatter dot plot of human TA selectivity vs Mouse TA selectivity. The population of points above the unity line indicates that the selectivity of the analogs for ERβ over ERα is greater for the human receptors when compared to the mouse receptors.

FIG. 5. ¹H NMR (CDCl₃, 400 MHz) of 1-(4-Methoxyphenyl)-12-heptyl-1,12-dicarba-closo-dodecaborane (Intermediate 1a).

FIG. 6. ¹H NMR (CDCl₃, 400 MHz) of 1-(4-methoxyphenyl)-12-(3-phenylpropyl)-1,12-dicarba-closododecaborane (intermediate 4a).

FIG. 7. ¹H NMR (CDCl₃, 400 MHz) of 1-(4-Hydroxyphenyl)-12-heptyl-1,12-dicarba-closo-dodecaborane (Compound 1).

FIG. 8. MS (APCI-) of 1-(4-Hydroxyphenyl)-12-heptyl-1,12-dicarba-closododecaborane (Compound 1).

FIG. 9. ¹H NMR (CDCl₃, 400 MHz) of 1-(4-hydroxyphenyl)-12-(3-phenylpropyl)-1,12-dicarba-closododecaborane (Compound 4).

FIG. 10. MS (APCI-) of 1-(4-hydroxyphenyl)-12-(3-phenylpropyl)-1,12-dicarba-closododecaborane (Compound 4).

FIG. 11. ¹H NMR (CDCl₃, 300 MHz) of 1-mercapto-12-(4-methoxyphenyl)-1,12-dicarba-closododecaborane.

FIG. 12. MS (APCI-) of 1-mercapto-12-(4-methoxyphenyl)-1,12-dicarba-closododecaborane.

FIG. 13. ¹H NMR (CDCl₃, 400 MHz) of 1-mercapto-12-(4-hydroxyphenyl)-1,12-dicarba-closododecaborane.

FIG. 14. MS (APCI-) of 1-mercapto-12-(4-hydroxyphenyl)-1,12-dicarba-closododecaborane.

FIG. 15. ¹H NMR (CDCl₃, 400 MHz) of 1-(4-hydroxyphenyl)-12-(hexylthio)-1,12-dicarba-closododecaborane (Compound 2).

FIG. 16. MS (APCI-) of 1-(4-hydroxyphenyl)-12-(hexylthio)-1,12-dicarba-closododecaborane (Compound 2).

FIG. 17. ¹H NMR (CDCl₃, 400 MHz) of 1-(4-hydroxyphenyl)-12-((2-(2-hydroxyethoxy)ethyl)thio)-1,12-dicarba-closododecaborane (Compound 3).

FIG. 18. MS (APCI-) of 1-(4-hydroxyphenyl)-12-((2-(2-hydroxyethoxy)ethyl)thio)-1,12-dicarba-closododecaborane (Compound 3).

FIG. 19. ¹H NMR (CDCl₃, 400 MHz) of 1-(4-hydroxyphenyl)-12-(phenethylthio)-1,12-dicarba-closododecaborane (Compound 5).

FIG. 20. MS (APCI-) of 1-(4-hydroxyphenyl)-12-(phenethylthio)-1,12-dicarba-closododecaborane (Compound 5).

FIG. 21. ¹H NMR (CDCl₃, 400 MHz) of 1-mercapto-7-(4-methoxyphenyl)-1,7-dicarba-closododecaborane.

FIG. 22. MS (APCI-) of 1-mercapto-7-(4-methoxyphenyl)-1,7-dicarba-closododecaborane.

FIG. 23. ¹H NMR (CDCl₃, 400 MHz) of 1-mercapto-7-(4-hydroxyphenyl)-1,7-dicarba-closododecaborane (Compound 11).

FIG. 24. MS (APCI-) of 1-mercapto-7-(4-hydroxyphenyl)-1,7-dicarba-closododecaborane (Compound 11).

FIG. 25. ¹H NMR (CDCl₃, 400 MHz) of 1-(4-hydroxyphenyl)-7-(propylthio)-1,7-dicarba-closododecaborane (Compound 7).

FIG. 26. MS (APCI-) of 1-(4-hydroxyphenyl)-7-(propylthio)-1,7-dicarba-closododecaborane (Compound 7).

FIG. 27. ¹H NMR (CDCl₃, 400 MHz) of 1-(4-hydroxyphenyl)-7-((2-hydroxyethyl)thio)-1,7-dicarba-closododecaborane (Compound 8).

FIG. 28. MS (APCI-) of 1-(4-hydroxyphenyl)-7-((2-hydroxyethyl)thio)-1,7-dicarba-closododecaborane (Compound 8).

FIG. 29. ¹H NMR (CDCl₃, 400 MHz) of 1-(4-hydroxyphenyl)-7-(methylthio)-1,7-dicarba-closododecaborane (Compound 10).

FIG. 30. MS (APCI-) of 1-(4-hydroxyphenyl)-7-(methylthio)-1,7-dicarba-closododecaborane (Compound 10).

FIG. 31. ¹H NMR (CDCl₃, 400 MHz) of 1-(4-hydroxyphenyl)-7-((2-hydroxyethyl)sulfonyl)-1,7-dicarba-closododecaborane (Compound 9).

FIG. 32. MS (APCI-) of 1-(4-hydroxyphenyl)-7-((2-hydroxyethyl)sulfonyl)-1,7-dicarba-closododecaborane (Compound 9).

FIG. 33. ¹H NMR (CDCl₃, 400 MHz) of 1-(4-methoxyphenyl)-7-(butyl)-1,7-dicarba-closododecaborane.

FIG. 34. ¹H NMR (CDCl₃, 400 MHz) of 1-(4-hydroxyphenyl)-7-(butyl)-1,7-dicarba-closododecaborane (Compound 6).

FIG. 35. MS (APCI-) of 1-(4-hydroxyphenyl)-7-(butyl)-1,7-dicarba-closododecaborane (Compound 6).

FIG. 36A-FIG. 36E. Curve fits for human ERα and ERβ transcriptional activation by para-substituted carborane analogs. The analogs were tested at several concentrations ranging from (0.1-30000 nm) on ERα or ERβ transfected HEK293 cells. Data are presented as mean±SEM. The EC₅₀ value is the ligand concentration of each analog at which half maximal receptor activity was observed. EC₅₀ values were derived from the curve fits using GraphPad Prism.

FIG. 37A-FIG. 37E. Curve fits for human ERα and ERβ transcriptional activation by meta-substituted carborane analogs. The analogs were tested at several concentrations ranging from (0.1-30000 nm) on ERα or ERβ overexpressing HEK293 cell lines. Data are presented as mean±SEM. The EC₅₀ value is the ligand concentration of each analog at which half maximal receptor activity was observed. EC₅₀ values were derived from the curve fits using GraphPad Prism.

FIG. 38A. Concentration time profile and NCA analysis for OSU-ERβ-12. Red and blue lines represent plasma concentration after oral and intravenous administration, respectively. Error bars represent mean±standard deviation.

FIG. 38B. Concentration time profile and NCA analysis for Compound 1. Red and blue lines represent plasma concentration after oral and intravenous administration, respectively. Error bars represent mean±standard deviation.

FIG. 38C. Concentration time profile and NCA analysis for Compound 2. Red and blue lines represent plasma concentration after oral and intravenous administration, respectively. Error bars represent mean±standard deviation.

FIG. 38D. Concentration time profile and NCA analysis for Compound 3. Red and blue lines represent plasma concentration after oral and intravenous administration, respectively. Error bars represent mean±standard deviation.

FIG. 38E. Concentration time profile and NCA analysis for Compound 4. Red and blue lines represent plasma concentration after oral and intravenous administration, respectively. Error bars represent mean±standard deviation.

FIG. 38F. Concentration time profile and NCA analysis for Compound 5. Red and blue lines represent plasma concentration after oral and intravenous administration, respectively. Error bars represent mean±standard deviation.

FIG. 39A. Concentration time profile and NCA analysis for Compound 6. Red and blue lines represent plasma concentration after oral and intravenous administration, respectively. Error bars represent mean±standard deviation.

FIG. 39B. Concentration time profile and NCA analysis for Compound 7. Red and blue lines represent plasma concentration after oral and intravenous administration, respectively. Error bars represent mean±standard deviation.

FIG. 39C. Concentration time profile and NCA analysis for Compound 8. Red and blue lines represent plasma concentration after oral and intravenous administration, respectively. Error bars represent mean±standard deviation.

FIG. 39D. Concentration time profile and NCA analysis for Compound 9. Red and blue lines represent plasma concentration after oral and intravenous administration, respectively. The pharmacokinetic profile of compound 9 after oral administration could not be fully determined due to plasma concentrations below the limit of quantification. Error bars represent mean standard deviation.

FIG. 39E. Concentration time profile and NCA analysis for Compound 10. Red and blue lines represent plasma concentration after oral and intravenous administration, respectively. Error bars represent mean±standard deviation.

FIG. 39F. Concentration time profile and NCA analysis for Compound 11. Red and blue lines represent plasma concentration after oral and intravenous administration, respectively. Error bars represent mean±standard deviation.

FIG. 40A-FIG. 40E. Scatter dot plots of blotted uterine weight as a percentage of body weight after administration of Estradiol (s.c 50 ug/kg) and para-substituted carborane analogs administered orally. The analogs were tested at several doses ranging from (3-300 mg/kg) in estrogen naïve female mice. Data are presented as mean±SD and were analyzed by One-way ANOVA in comparison with vehicle control. ****, ***, **, * correspond to p<0.0001, 0.001, 0.01 and 0.05, respectively.

FIG. 41A-FIG. 41E. Scatter dot plots of blotted uterine weight as a percentage of body weight after administration of Estradiol (s.c 50 ug/kg) and increasing doses of meta-substituted carborane analogs administered orally. The analogs were tested at several doses ranging from (3-300 mg/kg) in estrogen naïve female mice. Data are presented as mean SD and were analyzed by One-way ANOVA in comparison with vehicle control. ****, ***, ** correspond to p<0.0001, 0.001, 0.01 and 0.05, respectively.

FIG. 42A-FIG. 42E. Curve fits for mouse ERα and ERβ transcriptional activation by para-substituted carborane analogs. The analogs were tested at several concentrations ranging from (0.1-30000 nM) on ERα or ERβ transfected HEK293 cells. Data are presented as mean±SEM. The EC₅₀ value is the ligand concentration of each analog at which half maximal receptor activity was observed. EC₅₀ values were derived from the curve fits using GraphPad Prism as outlined in the materials and methods section.

FIG. 43A-FIG. 43E. Curve fits for mouse ERα and ERβ transcriptional activation by meta-substituted carborane analogs. The analogs were tested at several concentrations ranging from (0.1-30000 nM) on ERα on ERβ transfected HEK293 cells. Data are presented as mean±SEM. The EC₅₀ value is the ligand concentration of each analog at which half maximal receptor activity was observed. EC₅₀ values were derived from the curve fits using GraphPad Prism as outlined in the materials and methods section.

### DETAILED DESCRIPTION

The compounds, compositions, and methods described herein may be understood more readily by reference to the following detailed description of specific aspects of the disclosed subject matter and the Examples included therein.

Before the present compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “anticancer” refers to the ability to treat or control cellular proliferation and/or tumor growth at any concentration.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₅, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-phenoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

### Compounds

Disclosed herein are compounds comprising dicarba-closo-dodecaborane. Dicarba-closo-dodecaborane (also referred to herein as “carborane”) is an icosahedral cluster containing two carbon atoms and ten boron atoms in which both atoms are hexacoordinated. In carboranes, depending on the position of the carbon atoms in the cluster, 3 kinds of isomers exist, i.e., 1,2-dicarba-closo-dodecaborane (ortho-carborane), 1,7-dicarba-closo-dodecaborane (meta-carborane), and 1,12-dicarba-closo-dodecaborane (para-carborane). These structures are unique among boron compounds, as they can have high thermal stabilities and hydrophobicities, for example, comparable to hydrocarbons.

Carboranes can be used, for example, in ¹⁰Boron-Neutron Capture Therapy (BNCT). BNCT has been developed as a therapy for glioma and melanoma. When ¹⁰B is irradiated with thermal neutron (slow neutron), and a ray with 2.4 MeV energy is emitted and the atom decomposed to ⁷Li and ⁴He. The range of a ray is about 10 μm, which corresponds to the diameter of cells Therefore, effects are expected that only cells in which ¹⁰B atoms are uptaken are destroyed and other cells are not damaged. For the development of BNCT, it is important to have cancer cells selectively uptake ¹⁰B atoms in a concentration capable of destroying cells with neutron radiation. For that purpose, other-carborane skeleton has been utilized which has been utilized which has low toxicity and a high ¹⁰B content, and is easy to be synthesized. Moreover, nucleic acid precursors, amino acids, and porphyrins which contain ortho-carboranes have been synthesized and subjected to evaluation.

Carborane-based ERβ agonists and carborane analogs are described, for example, in U.S. Pat. No. 6,838,574 to Endo, U.S. Patent Application Publication No. 2018/0264017 to Tjarks et al., and PCT/US2019/064228 to Coss et al., each of which is hereby incorporated by reference in its entirety.

Disclosed herein are compounds comprising dicarba-closo-dodecaborane. The compounds can be, for example, estrogen receptor beta (ERβ) agonists. In some examples, the compounds can be selective ERβ agonists.

In some examples, the compounds are defined by Formula I, or a pharmaceutically acceptable salt thereof:

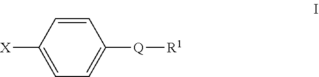

wherein
Q is a substituted or unsubstituted dicarba-closo-dodecaborane cluster, and

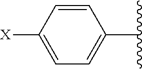

and R¹ are attached to Q in a meta configuration;
X is OH, NHR², SH, or S(O)(O)NHR²;
R¹ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)NR³R⁴, or NR³R⁴;
R² is H, OH, halogen, or substituted or unsubstituted C₁-C₄ alkyl;
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl.

In some examples of Formula I, Q is

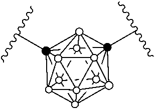

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples, the compound is defined by Formula II, or a pharmaceutically acceptable salt thereof:

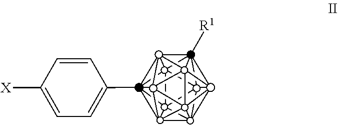

wherein
● is a carbon atom;
◯ is B—H, B-halogen, B-alkyl, B—OH, or B—NH₂;
X is OH, NHR², SH, or S(O)(O)NHR²;
R¹ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)NR³R⁴, or NR³R⁴;
R² is H, OH, halogen, or substituted or unsubstituted C₁-C₄ alkyl;
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl.

In some examples of Formula I and/or Formula II, X is OH.

In some examples of Formula I and/or Formula II, when X is OH, R¹ is not (CH₂)₅CH(CH₃)₂ or NH₂.

In some examples of Formula I and/or Formula II, R¹ is substituted or unsubstituted C₁-C₂₀ alkyl. In some examples of Formula I and/or Formula II, X is OH and R¹ is substituted or unsubstituted C₁-C₂₀ alkyl. In some examples of Formula II, X is OH; R¹ is substituted or unsubstituted C₁-C₂₀ alkyl; ● is a carbon atom and ◯ is B—H.

In some examples of Formula I and/or Formula II, R¹ is C₁-C₂₀ alkyl, optionally substituted with OH, sulfonyl, thiol, or a combination thereof. In some examples of Formula I and/or Formula II, X is OH and R¹ is C₁-C₂₀ alkyl, optionally substituted with OH, sulfonyl, thiol, or a combination thereof. In some examples of Formula II, X is OH; R¹ is C₁-C₂₀ alkyl, optionally substituted with OH, sulfonyl, thiol, or a combination thereof, ● is a carbon atom and
◯ is B—H.

In some examples of Formula I and/or Formula II, R¹ is an unsubstituted C₁-C₂₀ alkyl. In some examples of Formula I and/or Formula II, X is OH and R¹ is an unsubstituted C₁-C₂₀ alkyl. In some examples of Formula II, X is OH; R¹ is an unsubstituted C₁-C₂₀ alkyl; ● is a carbon atom and ◯ is B—H.

In some examples of Formula I and/or Formula II, R¹ is a substituted or unsubstituted C₁-C₁₀ alkyl. In some examples of Formula I and/or Formula II, X is OH and R¹ is a substituted or unsubstituted C₁-C₁₀ alkyl. In some examples of Formula II, X is OH; R¹ is a substituted or unsubstituted C₁-C₁₀ alkyl; ● is a carbon atom and ◯ is B—H.

In some examples of Formula I and/or Formula II, R¹ is C₁-C₁₀ alkyl, optionally substituted with OH, sulfonyl, thiol, or a combination thereof. In some examples of Formula I and/or Formula II, X is OH and R¹ is C₁-C₁₀ alkyl, optionally substituted with OH, sulfonyl, thiol, or a combination thereof. In some examples of Formula II, X is OH; R¹ is C₁-C₁₀ alkyl, optionally substituted with OH, sulfonyl, thiol, or a combination thereof, ● is a carbon atom and ◯ is B—H.

In some examples of Formula I and/or Formula II, R¹ is an unsubstituted C₁-C₁₀ alkyl, such as an unsubstituted C₁-C₆ alkyl. In some examples of Formula I and/or Formula II, X is OH and R¹ is an unsubstituted C₁-C₁₀ alkyl, such as an unsubstituted C₁-C₆ alkyl. In some examples of Formula II, X is OH; R¹ is an unsubstituted C₁-C₁₀ alkyl, such as an unsubstituted C₁-C₆ alkyl; ● is a carbon atom and ◯ is B—H.

In some examples of Formula I and/or Formula II, R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₄-C₂₀ alkylcycloalkyl, substituted or unsubstituted C₁-C₂₀ acyl, C₁-C₂₀ acyl, —C(O)NR³R⁴, or NR³R⁴.

In some examples of Formula I and/or Formula II, R¹ is substituted or unsubstituted C₄-C₂₀ alkyl. In some examples of Formula I and/or Formula II, X is OH and R¹ is substituted or unsubstituted C₄-C₂₀ alkyl. In some examples of Formula II, X is OH; R¹ is substituted or unsubstituted C₄-C₂₀ alkyl; ● is a carbon atom and ◯ is B—H.

In some examples of Formula I and/or Formula II, R¹ is C₄-C₂₀ alkyl, optionally substituted with OH, sulfonyl, thiol, or a combination thereof. In some examples of Formula I and/or Formula II, X is OH and R¹ is C₄-C₂₀ alkyl, optionally substituted with OH, sulfonyl, thiol, or a combination thereof. In some examples of Formula II, X is OH; R¹ is C₄-C₂₀ alkyl, optionally substituted with OH, sulfonyl, thiol, or a combination thereof, ● is a carbon atom and
◯ is B—H.

In some examples of Formula I and/or Formula II, R¹ is an unsubstituted C₄-C₂₀ alkyl. In some examples of Formula I and/or Formula II, X is OH and R¹ is an unsubstituted C₄-C₂₀ alkyl. In some examples of Formula II, X is OH; R¹ is an unsubstituted C₄-C₂₀ alkyl; ● is a carbon atom and ◯ is B—H.

In some examples of Formula I and/or Formula II, R¹ is a substituted or unsubstituted C₄-C₁₀ alkyl. In some examples of Formula I and/or Formula II, X is OH and R¹ is a substituted or unsubstituted C₄-C₁₀ alkyl. In some examples of Formula II, X is OH; R¹ is a substituted or unsubstituted C₄-C₁₀ alkyl; ● is a carbon atom and ◯ is B—H.

In some examples of Formula I and/or Formula II, R¹ is C₄-C₁₀ alkyl, optionally substituted with OH, sulfonyl, thiol, or a combination thereof. In some examples of Formula I and/or Formula II, X is OH and R¹ is C₄-C₁₀ alkyl, optionally substituted with OH, sulfonyl, thiol, or a combination thereof. In some examples of Formula II, X is OH; R¹ is C₄-C₁₀ alkyl, optionally substituted with OH, sulfonyl, thiol, or a combination thereof, ● is a carbon atom and ◯ is B—H.

In some examples of Formula I and/or Formula II, R¹ is an unsubstituted C₄-C₁₀ alkyl, such as an unsubstituted C₄-C₆ alkyl. In some examples of Formula I and/or Formula II, X is OH and R¹ is an unsubstituted C₄-C₁₀ alkyl, such as an unsubstituted C₄-C₆ alkyl. In some examples of Formula II, X is OH; R¹ is an unsubstituted C₄-C₁₀ alkyl, such as an unsubstituted C₄-C₆ alkyl; ● is a carbon atom and ◯ is B—H.

In some examples of Formula I and/or Formula II, R¹ is

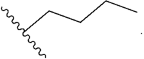 [SMILES: *CCCC]

In some examples of Formula I and/or Formula II, X is OH and R¹ is

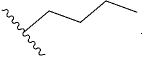 [SMILES: *CCCC]

In some examples of Formula II, X is OH; R¹ is

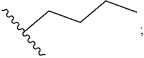 [SMILES: *CCCC]

● is a carbon atom and ◯ is B—H.

In some examples of Formula I and/or Formula II, R¹ is a substituted or unsubstituted C₆-C₁₀ alkyl. In some examples of Formula I and/or Formula II, R¹ is a C₆-C₁₀ hydroxyalkyl.

In some examples of Formula I and/or Formula II, R¹ is a substituted or unsubstituted C₃-C₁₆ alkylaryl. In some examples of Formula I and/or Formula II, R¹ is a C₃-C₁₆ hydroxyalkylaryl.

In some examples of Formula I and/or Formula II, R¹ is a substituted or unsubstituted C₈-C₂₀ alkylaryl. In some examples of Formula I and/or Formula II, R¹ is a C₈-C₂₀ hydroxyalkylaryl.

In some examples of Formula I and/or Formula II, R¹ is a substituted or unsubstituted C₅-C₁₀ acyl.

In some examples of Formula I and/or Formula II, R¹ is a substituted or unsubstituted branched C₄-C₁₀ alkyl. In some examples of Formula I and/or Formula II, R¹ is a branched C₄-C₁₀ hydroxyalkyl.

In some examples, the compound is defined by Formula III, or a pharmaceutically acceptable salt thereof:

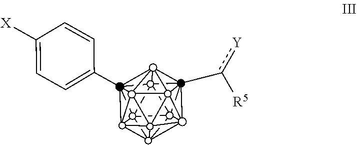

wherein
● is a carbon atom;
◯ is B—H, B-halogen, B-alkyl, B—OH, or B—NH₂;
the dotted line to Y indicates that the bond can be a single bond or a double bond, as valence permits;
X is OH, NHR², SH, or S(O)(O)NHR²;
Y is O, OR^{2′}, NHR², SH, or S(O)(O)NHR²;
R⁵ is substituted or unsubstituted C₂-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, or substituted or unsubstituted C₃-C₁₉ alkylcycloalkyl, or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula III, X is OH.

In some examples of Formula III, Y is OH.

In some examples of Formula III, Y is O.

In some examples of Formula III, R⁵ is a substituted or unsubstituted C₃-C₉ alkyl.

In some examples of Formula III, R⁵ is a substituted or unsubstituted C₆-C₉ alkyl.

In some examples of Formula III, R⁵ is a substituted or unsubstituted C₂-C₁₅ alkylaryl.

In some examples of Formula III, R⁵ is a substituted or unsubstituted branched C₂-C₉ alkyl.

In some examples, the compound is defined by Formula IV, or a pharmaceutically acceptable salt thereof:

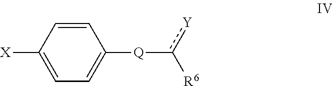

wherein
Q is a substituted or unsubstituted dicarba-closo-dodecaborane cluster, and

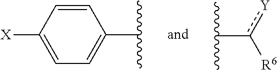

are attached to Q in a meta configuration;
the dotted line to Y indicates that the bond can be a single bond or a double bond, as valence permits;
X is OH, NHR², SH, or S(O)(O)NHR²;
Y is O, OR^{2′}, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl, or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula IV, Q is

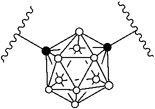

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples, the compound is defined by Formula V, or a pharmaceutically acceptable salt thereof:

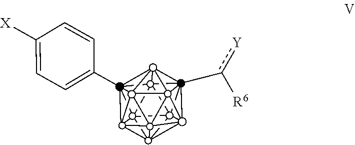

wherein
● is a carbon atom;
◯ is B—H, B-halogen, B-alkyl, B—OH, or B—NH₂;
the dotted line to Y indicates that the bond can be a single bond or a double bond, as valence permits;
X is OH, NHR², SH, or S(O)(O)NHR²;
Y is O, OR^{2′}, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl, or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula IV and/or Formula V, X is OH.

In some examples of Formula IV and/or Formula V, when X is OH, R⁶ is not CH₂OH, CH(CH₃)OH, CH₂CH₂OH, CH₂CH₂CH₂OH, (CH₂)₅CH(CH₃)₂, or NH₂.

In some examples of Formula IV and/or Formula V, Y is OH.

In some examples of Formula IV and/or Formula V, Y is O.

In some examples of Formula IV and/or Formula V, R⁶ is a substituted or unsubstituted C₆-C₁₀ alkyl.

In some examples of Formula IV and/or Formula V, R⁶ is a substituted or unsubstituted C₂-C₁₅ alkylaryl.

In some examples of Formula IV and/or Formula V, R⁶ is a substituted or unsubstituted C₈-C₂₀ alkylarylcycloalkyl.

In some examples of Formula IV and/or Formula V, R⁶ is a substituted or unsubstituted branched C₃-C₁₀ alkyl.

In some examples, the compound is defined by Formula VI, or a pharmaceutically acceptable salt thereof:

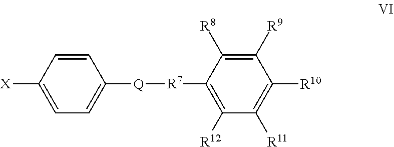

wherein
Q is a substituted or unsubstituted dicarba-closo-dodecaborane cluster, and

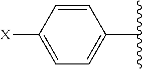

and R⁷ are attached to Q in a meta configuration;
X is OH, NHR², SH, or S(O)(O)NHR²;

R⁷ is substituted or unsubstituted C₁-C₁₄ alkyl, substituted or unsubstituted C₂-C₁₄ alkenyl, substituted or unsubstituted C₂-C₁₄ alkynyl, substituted or unsubstituted C₁-C₁₄ acyl, or NR³R⁴;
R¹, R⁹, R¹⁰, R¹¹, and R¹² are independently H, OH, halogen, substituted or unsubstituted C₁-C₂₀ alkyl, sub substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, substituted or unsubstituted C₁-C₂₀ acyl, or NR³R⁴, or wherein, as valence permits, R¹ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula VI, Q is

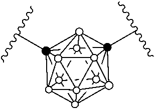

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples, the compound is defined by Formula VII, or a pharmaceutically acceptable salt thereof:

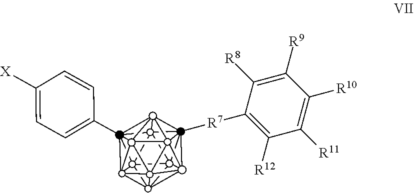

wherein
● is a carbon atom;
◯ is B—H, B-halogen, B-alkyl, B—OH, or B—NH₂;
X is OH, NHR², SH, or S(O)(O)NHR²;
R⁷ is substituted or unsubstituted C₁-C₁₄ alkyl, substituted or unsubstituted C₂-C₁₄ alkenyl, substituted or unsubstituted C₂-C₁₄ alkynyl, substituted or unsubstituted C₁-C₁₄ acyl, or NR³R⁴;
R¹, R⁹, R¹⁰, R¹¹, and R¹² are independently H, OH, halogen, substituted or unsubstituted C₁-C₂₀ alkyl, sub substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, substituted or unsubstituted C₁-C₂₀ acyl, or NR³R⁴, or wherein, as valence permits, R¹ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula VI and/or Formula VII, X is OH.

In some examples of Formula VI and/or Formula VII, R⁷ is a substituted or unsubstituted C₁-C₇ alkyl.

In some examples of Formula VI and/or Formula VII, R⁷ is a C₁-C₇ hydroxyalkyl.

In some examples of Formula VI and/or Formula VII, R⁸-R¹² are independently H, OH, halogen, or substituted or unsubstituted C₁-C₄ alkyl, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms.

In some examples of Formula VI and/or Formula VII, R⁸-R¹² are each H.

In some examples of Formula VI and/or Formula VII, R⁸, R¹⁰, and R¹² are each H, and R⁹ and R¹⁰, together with the atoms to which they are attached, form a substituted or unsubstituted 5-7 membered cyclic moiety.

In some examples, the compound is of Formula VIII, or a pharmaceutically acceptable salt thereof:

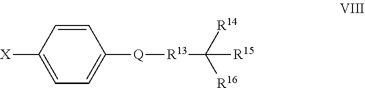

wherein
Q is a substituted or unsubstituted dicarba-closo-dodecaborane cluster, and

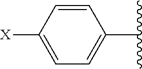

and R¹³ are attached to Q in a meta configuration;
X is OH, NHR², SH, or S(O)(O)NHR²;
R¹³ is substituted or unsubstituted C₁-C₁₉ alkyl, substituted or unsubstituted C₂-C₁₉ alkenyl, substituted or unsubstituted C₂-C₁₉ alkynyl, or substituted or unsubstituted C₁-C₂₀ acyl; and
R¹⁴, R¹⁵, and R¹⁶ are independently hydrogen, halogen, hydroxyl, substituted or unsubstituted C₁-C₁₈ alkyl, substituted or unsubstituted C₂-C₁₈ alkenyl, substituted or unsubstituted C₁-C₁₈ alkynyl, substituted or unsubstituted C₂-C₁₈ aryl, substituted or unsubstituted C₃-C₁₈ cycloalkyl, substituted or unsubstituted C₁-C₂₀ acyl, or NR³R⁴, or wherein, as valence permits, R¹⁴ and R¹⁵, R¹⁴ and R¹⁶, or R¹⁵ and R¹⁶, together with the atoms to which they are attached, for a 3-10 membered substituted or unsubstituted cyclic moiety optionally including from 1 to 3 heteroatoms,
with the proviso that at least two of R¹⁴, R¹⁵ and R¹⁶ are not hydrogen, halogen, or hydroxyl; and
with the proviso that when X is OH and R¹³ is a C₅ alkyl, R¹⁴, R¹⁵, and R¹⁶ are not H, methyl, and methyl.

In some examples of Formula VIII, Q is

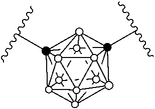

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples, the compound is defined by Formula IX, or a pharmaceutically acceptable salt thereof:

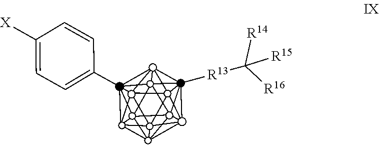

wherein
● is a carbon atom;
◯ is B—H, B-halogen, B-alkyl, B—OH, or B—NH₂;
X is OH, NHR², SH, or S(O)(O)NHR²;
R¹³ is substituted or unsubstituted C₁-C₁₉ alkyl, substituted or unsubstituted C₂-C₁₉ alkenyl, substituted or unsubstituted C₂-C₁₉ alkynyl, or substituted or unsubstituted C₁-C₂₀ acyl; and
R¹⁴, R¹⁵, and R¹⁶ are independently hydrogen, halogen, hydroxyl, substituted or unsubstituted C₁-C₁₈ alkyl, substituted or unsubstituted C₂-C₁₈ alkenyl, substituted or unsubstituted C₁-C₁₈ alkynyl, substituted or unsubstituted C₂-C₁₈ aryl, substituted or unsubstituted C₃-C₁₈ cycloalkyl, substituted or unsubstituted C₁-C₂₀ acyl, or NR³R⁴, or wherein, as valence permits, R¹⁴ and R¹⁵, R¹⁴ and R¹⁶, or R¹⁵ and R¹⁶, together with the atoms to which they are attached, for a 3-10 membered substituted or unsubstituted cyclic moiety optionally including from 1 to 3 heteroatoms,
with the proviso that at least two of R¹⁴, R¹⁵ and R¹⁶ are not hydrogen, halogen, or hydroxyl; and
with the proviso that when X is OH and R¹³ is a C₅ alkyl, R¹⁴, R¹⁵, and R¹⁶ are not H, methyl, and methyl.

In some examples of Formula VIII and/or Formula IX, X is OH.

In some examples of Formula VIII and/or Formula IX, R¹³ is a substituted or unsubstituted C₄-C₈ alkyl. In some examples of Formula VIII and/or Formula IX, R¹³ is a C₄-C₈ hydroxyalkyl.

In some examples of Formula VIII and/or Formula IX, R¹⁴-R¹⁶ are independently hydrogen, halogen, hydroxyl, substituted or unsubstituted C₁-C₄ alkyl, with the proviso that at least two of R¹⁴, R¹⁵ and R¹⁶ are not hydrogen, halogen, or hydroxyl; and with the proviso that when X is OH and R¹³ is a C₅ alkyl, R¹⁴, R¹⁵, and R¹⁶ are not H, methyl, and methyl.

In some examples, the compound defined by Formula XI, or a pharmaceutically acceptable salt thereof

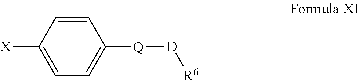

wherein
Q is a substituted or unsubstituted dicarba-closo-dodecaborane cluster, and

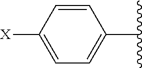

and D are attached to Q in a meta configuration;
D is —S—, —S(O)—, —S(O)(O)—, —S(O)(NH)—, —P(O)(OH)O—, —P(O)(OH)NH—, or —O—;
X is OH, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, or substituted or unsubstituted C₄-C₂₀ alkylheterocycloalkyl; and
R² is H, OH, halogen, or substituted or unsubstituted C₁-C₄ alkyl.

In some examples of Formula XI, Q is

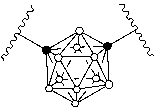

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples, the compound is defined by Formula XIA, or a pharmaceutically acceptable salt thereof:

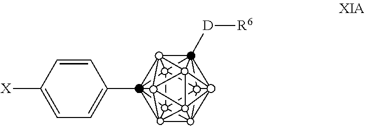

wherein
● is a carbon atom or a boron atom; and
◯ is C—H, C-halogen, C-alkyl, C—OH, C—NH₂, B—H, B-halogen, B-alkyl, B—OH, or B—NH₂
D is —S—, —S(O)—, —S(O)(O)—, —S(O)(NH)—, —P(O)(OH)O—, —P(O)(OH)NH—, or —O—;
X is OH, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, or substituted or unsubstituted C₄-C₂₀ alkylheterocycloalkyl; and
R² is H, OH, halogen, or substituted or unsubstituted C₁-C₄ alkyl.

In some examples of Formula XI and/or Formula XIA, X is OH.

In some examples of Formula XI and/or Formula XIA, D is —S—, or —S(O)(O)—. In some examples of Formula XI and/or Formula XIA, X is OH and D is —S—, or —S(O)(O)—. In some examples of Formula XIA, X is OH; D is —S—, or —S(O)(O)—; ● is a carbon atom and ◯ is B—H.

In some examples of Formula XI and/or Formula XIA, R⁶ is substituted or unsubstituted C₁-C₂₀ alkyl. In some examples of Formula XI and/or Formula XIA, X is OH and R⁶ is substituted or unsubstituted C₁-C₂₀ alkyl. In some examples of Formula XI and/or Formula XIA, X is OH; D is —S—, or —S(O)(O)—; and R⁶ is substituted or unsubstituted C₁-C₂₀ alkyl. In some examples of Formula XIA, X is OH; D is —S—, or —S(O)(O)—; R⁶ is substituted or unsubstituted C₁-C₂₀ alkyl; ● is a carbon atom and ◯ is B—H.

In some examples of Formula XI and/or Formula XIA, R⁶ is C₁-C₂₀ alkyl optionally substituted with OH. In some examples of Formula XI and/or Formula XIA, X is OH and R⁶ is C₁-C₂₀ alkyl optionally substituted with OH. In some examples of Formula XI and/or Formula XIA, X is OH; D is —S—, or —S(O)(O)—; and R⁶ is C₁-C₂₀ alkyl optionally substituted with OH. In some examples of Formula XIA, X is OH; D is —S—, or —S(O)(O)—; R⁶ is C₁-C₂₀ alkyl optionally substituted with OH; ● is a carbon atom and ◯ is B—H.

In some examples of Formula XI and/or Formula XIA, R⁶ is substituted or unsubstituted C₁-C₆ alkyl. In some examples of Formula XI and/or Formula XIA, X is OH and R⁶ is substituted or unsubstituted C₁-C₆ alkyl. In some examples of Formula XI and/or Formula XIA, X is OH; D is —S—, or —S(O)(O)—; and R⁶ is substituted or unsubstituted C₁-C₆ alkyl. In some examples of Formula XIA, X is OH; D is —S—, or —S(O)(O)—; and R⁶ is substituted or unsubstituted C₁-C₆ alkyl; ● is a carbon atom and ◯ is B—H.

In some examples of Formula XI and/or Formula XIA, R⁶ is substituted or unsubstituted C₁-C₃ alkyl. In some examples of Formula XI and/or Formula XIA, X is OH and R⁶ is substituted or unsubstituted C₁-C₃ alkyl. In some examples of Formula XI and/or Formula XIA, X is OH; D is —S—, or —S(O)(O)—; and R⁶ is substituted or unsubstituted C₁-C₃ alkyl. In some examples of Formula XIA, X is OH; D is —S—, or —S(O)(O)—; and R⁶ is substituted or unsubstituted C₁-C₃ alkyl; ● is a carbon atom and ◯ is B—H.

In some examples of Formula XI and/or Formula XIA, R⁶ is unsubstituted C₁-C₃ alkyl. In some examples of Formula XI and/or Formula XIA, X is OH and R⁶ is unsubstituted C₁-C₃ alkyl. In some examples of Formula XI and/or Formula XIA, X is OH; D is —S—, or —S(O)(O)—; and R⁶ is unsubstituted C₁-C₃ alkyl. In some examples of Formula XIA, X is OH; D is —S—, or —S(O)(O)—; R⁶ is unsubstituted C₁-C₃ alkyl; ● is a carbon atom and ◯ is B—H.

In some examples of Formula XI and/or Formula XIA, R⁶ is C₁-C₃ alkyl substituted with OH. In some examples of Formula XI and/or Formula XIA, X is OH and R⁶ is C₁-C₃ alkyl substituted with OH. In some examples of Formula XI and/or Formula XIA, X is OH; D is —S—, or —S(O)(O)—; and R⁶ is C₁-C₃ alkyl substituted with OH. In some examples of Formula XIA, X is OH; D is —S—, or —S(O)(O)—; R⁶ is C₁-C₃ alkyl substituted with OH; ● is a carbon atom and ◯ is B—H.

In some examples of Formula XI and/or Formula XIA, R⁶ is selected from the group consisting of

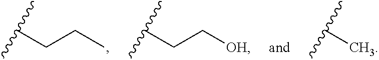

In some examples of Formula XI and/or Formula XIA, X is OH and R⁶ is selected from the group consisting of

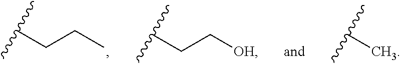

In some examples of Formula XI and/or Formula XIA, X is OH; D is —S—, or —S(O)(O)—; and R⁶ is selected from the group consisting of

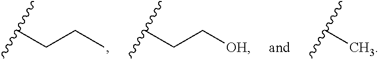

In some examples of Formula XIA, X is OH; D is —S—, or —S(O)(O)—; R⁶ is selected from the group consisting of

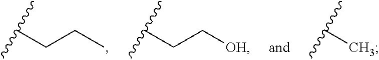

● is a carbon atom and ◯ is B—H.

In some examples of Formula XI and/or Formula XIA, R⁶ is selected from the group consisting of

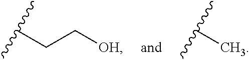

In some examples of Formula XI and/or Formula XIA, X is OH and R⁶ is selected from the group consisting of

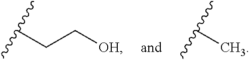

In some examples of Formula XI and/or Formula XIA, X is OH; D is —S—, or —S(O)(O)—; and R⁶ is selected from the group consisting of

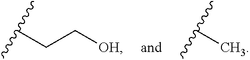

In some examples of Formula XIA, X is OH; D is —S—, or —S(O)(O)—; R⁶ is selected from the group consisting of

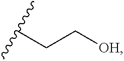 [SMILES: *CCO]

and; ● is a carbon atom and ◯ is B—H.

In some examples of Formula XI and/or Formula XIA, R⁶ is

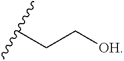 [SMILES: *CCO]

In some examples of Formula XI and/or Formula XIA, X is OH and R⁶ is

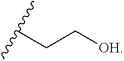 [SMILES: *CCO]

In some examples of Formula XI and/or Formula XIA, X is OH; D is —S—, or —S(O)(O)—; and R⁶ is

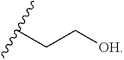 [SMILES: *CCO]

In some examples of Formula XIA, X is OH; D is —S—, or —S(O)(O)—; R⁶ is

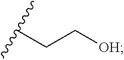 [SMILES: *CCO]

● is a carbon atom and ◯ is B—H.

In some examples of Formula XI and/or Formula XIA, D-R⁶ is selected from the group consisting of ●

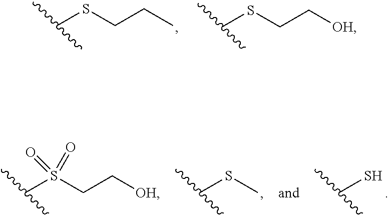

In some examples of Formula XI and/or Formula XIA, X is OH and D-R⁶ is selected from the group consisting of

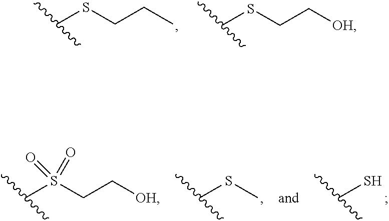

In some examples of Formula XIA, X is OH; D-R⁶ is selected from the group consisting of

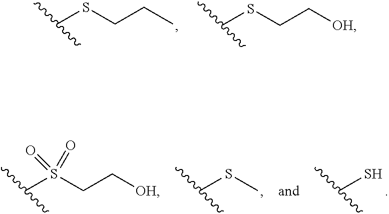

is a carbon atom and ◯ is B—H.

In some examples of Formula XI and/or Formula XIA, D-R⁶ is selected from the group consisting of

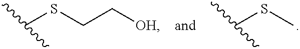

In some examples of Formula XI and/or Formula XIA, X is OH and D-R⁶ is selected from the group consisting of

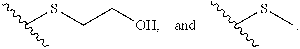

In some examples of Formula XIA, X is OH; D-R⁶ is selected from the group consisting of

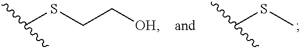

● is a carbon atom and ◯ is B—H.

In some examples of Formula XI and/or Formula XIA, D-R⁶ is

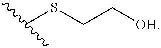 [SMILES: *SCCO]

In some examples of Formula XI and/or Formula XIA, X is OH and D-R⁶ is

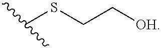 [SMILES: *SCCO]

In some examples of Formula XIA, X is OH; D-R⁶ is

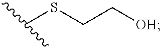 [SMILES: *SCCO]

● is a carbon atom and ◯ is B—H.

In some examples of Formula XI, R⁶ is a substituted or unsubstituted C₃-C₁₀ alkyl, such as a substituted or unsubstituted C₆-C₉ alkyl.

In some examples of Formula XI, R⁶ is a substituted or unsubstituted C₂-C₁₅ alkylaryl.

In some examples of Formula XI, R⁶ is a substituted or unsubstituted branched C₂-C₉ alkyl.

In some examples of Formula XI, R⁶ is a substituted or unsubstituted C₃-C₁₀ heteroalkyl, such as a substituted or unsubstituted C₆-C₉ heteroalkyl.

In some examples, the compound is defined by Formula XII, or a pharmaceutically acceptable salt thereof

A-Q-R¹ Formula XII

wherein
Q is a substituted or unsubstituted dicarba-closo-dodecaborane cluster, and A and R¹ are attached to Q in a meta configuration;
A is a substituted or unsubstituted heteroaryl ring;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some examples, the carborane or carborane analog comprises a compound defined by Formula XIIA, or a pharmaceutically acceptable salt thereof

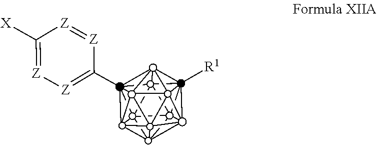

wherein
● is a carbon atom;
◯ is B—H, B-halogen, B-alkyl, B—OH, or B—NH₂;
X is OH, NHR², SH, or S(O)(O)NHR²;
Z is, individually for each occurrence, N or CH, with the proviso that at least one of Z is N;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some examples of Formula XIIA, the carborane or carborane analog comprises a compound defined by one of the formulae below, or a pharmaceutically acceptable salt thereof:

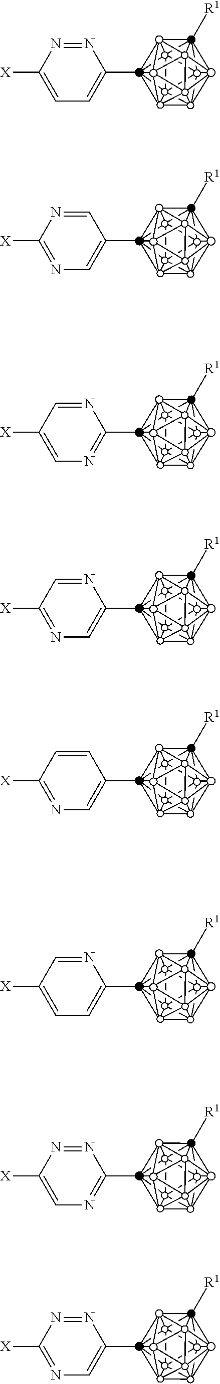

wherein
● is a carbon atom;
◯ is B—H, B-halogen, B-alkyl, B—OH, or B—NH₂;
X is OH, NHR², SH, or S(O)(O)NHR²;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some examples, the carborane or carborane analog comprises a compound defined by one of Formula XIIB-XIIF, or a pharmaceutically acceptable salt thereof:

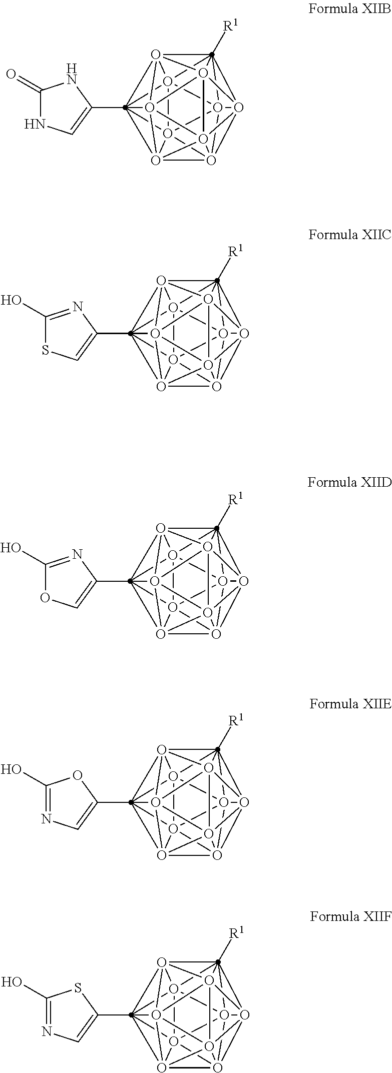

wherein
● is a carbon atom;
◯ is B—H, B-halogen, B-alkyl, B—OH, or B—NH₂;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some examples of Formula XIIA, X is OH.

In some examples of Formula XII and/or Formula XIIA-XIIF, R¹ is a substituted or unsubstituted C₆-C₁₀ alkyl. In some examples of Formula XII and/or Formula XIIA-XIIF, R¹ is a C₆-C₁₀ hydroxyalkyl.

In some examples of Formula XII and/or Formula XIIA-XIIF, R¹ is a substituted or unsubstituted C₃-C₁₆ alkylaryl. In some examples of Formula XII and/or Formula XIIA-XIIF, R¹ is a C₃-C₁₆ hydroxyalkylaryl.

In some examples of Formula XII and/or Formula XIIA-XIIF, R¹ is a substituted or unsubstituted C₈-C₂₀ alkylaryl. In some examples of Formula XII and/or Formula XIIA-XIIF, R¹ is a C₈-C₂₀ hydroxyalkylaryl.

In some examples of Formula XII and/or Formula XIIA-XIIF, R¹ is a substituted or unsubstituted C₅-C₁₀ acyl.

In some examples of Formula XII and/or Formula XIIA-XIIF, R¹ is a substituted or unsubstituted branched C₄-C₁₀ alkyl. In some examples of Formula XII and/or Formula XIIA-XIIF, R¹ is a branched C₄-C₁₀ hydroxyalkyl.

In some examples, the compound defined by one of the formulae below, or a pharmaceutically acceptable salt thereof:

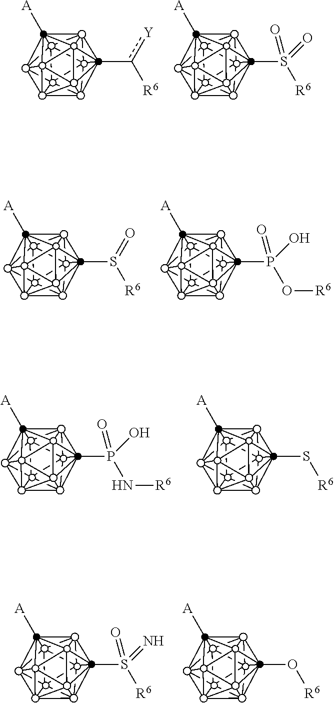

wherein
● is a carbon atom;
◯ is B—H, B-halogen, B-alkyl, B—OH, or B—NH₂;
the dotted line to Y indicates that the bond can be a single bond or a double bond, as valence permits;
A is a substituted or unsubstituted heteroaryl ring;
Y, when present, is O, halogen, OR^{2′}, NHR², SH, or S(O)(O)NHR²;
R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl. or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some examples, A is a five-membered substituted or unsubstituted heteroaryl ring, such as a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring.

In some examples, A is a six-membered substituted or unsubstituted heteroaryl ring, such as a pyridyl, pyrazinyl, pyrimidinyl, triazinyl, or pyridazinyl ring.

In some examples, R⁶ is a substituted or unsubstituted C₃-C₁₀ alkyl, such as a substituted or unsubstituted C₆-C₉ alkyl.

In some examples, R⁶ is a substituted or unsubstituted C₂-C₁₅ alkylaryl.

In some examples, R⁶ is a substituted or unsubstituted branched C₂-C₉ alkyl.

In some examples, R⁶ is a substituted or unsubstituted C₃-C₁₀ heteroalkyl, such as a substituted or unsubstituted C₆-C₉ heteroalkyl.

In some examples, the compound is selected from the group consisting of

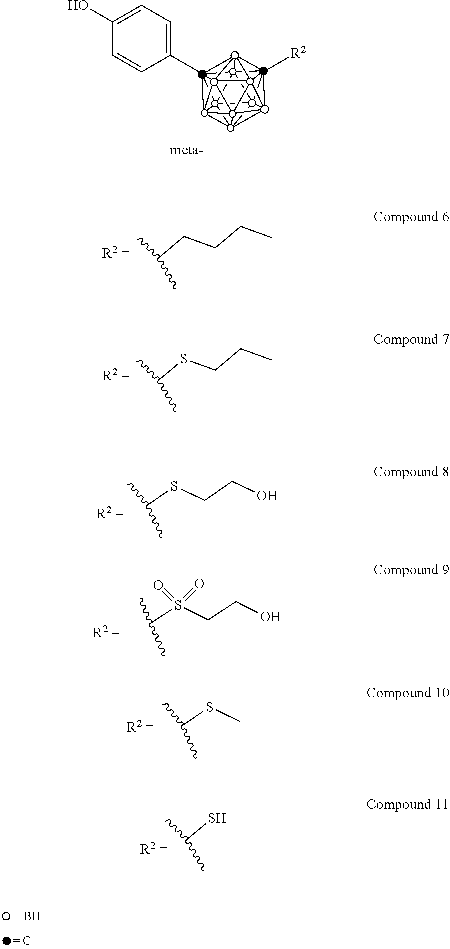

and pharmaceutically acceptable salts thereof.

In some examples, the compound comprises:

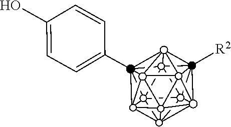

wherein
● is a carbon atom;
◯ is B—H; and
R² is selected from the group consisting of:

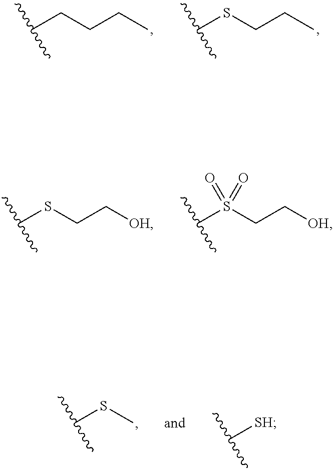

and pharmaceutically acceptable salts thereof.

In some examples, the compound is selected from the group consisting of:

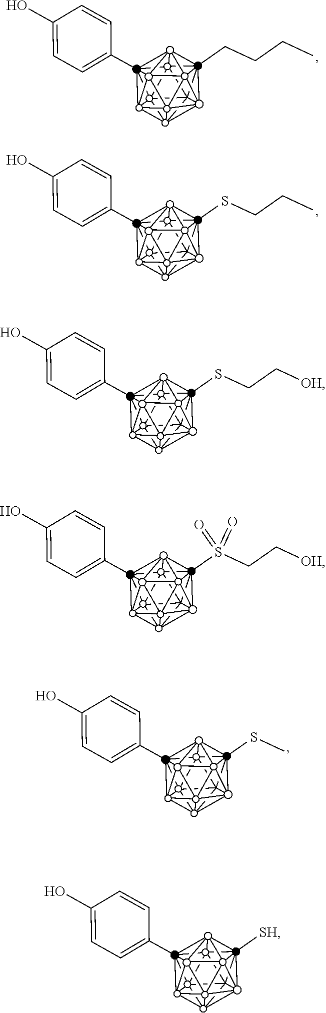

and pharmaceutically acceptable salts thereof, where ● is a carbon atom, and ◯ is B—H.

In some examples, the compound is selected from the group consisting of:

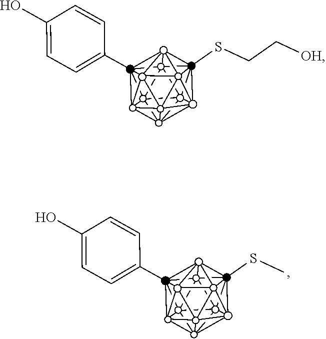

and pharmaceutically acceptable salts thereof, where ● is a carbon atom, and ◯ is B—H.

In some examples, The compound of any one of claims 32-34, wherein the compound is:

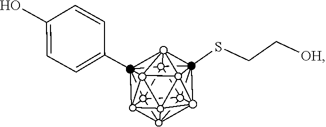

or a pharmaceutically acceptable salt thereof, where ● is a carbon atom, and ◯ is B—H.

In some examples, the compound has an EC₅₀ of 800 nM or less at estrogen receptor beta (ERβ) (e.g., 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 9 nM or less, 8 nM or less, 7 nM or less, 6 nM or less, 5 nM or less, 4.5 nM or less, 4 nM or less, 3.5 nM or less, 3 nM or less, 2.5 nM or less, 2 nM or less, 1.5 nM or less, 1 nM or less, 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less). In some examples, the compound has an EC₅₀ of 6 nM or less at estrogen receptor beta (ERβ). In some examples, the compound has an EC₅₀ of 200 nM or less at estrogen receptor beta (ERβ). In some examples, the compound has an EC₅₀ of 200 nM or less at estrogen receptor beta (ERβ) in both a human model and a mouse model.

In some examples, the compounds disclosed herein can have an EC₅₀ of 1 pM or more at ERβ (e.g., 0.1 nM or more, 0.2 nM or more, 0.3 nM or more, 0.4 nM or more, 0.5 nM or more, 0.6 nM or more, 0.7 nM or more, 0.8 nM or more, 0.9 nM or more, 1 nM or more, 1.5 nM or more, 2 nM or more, 2.5 nM or more, 3 nM or more, 3.5 nM or more, 4 nM or more, 4.5 nM or more, 5 nM or more, 6 nM or more, 7 nM or more, 8 nM or more, 9 nM or more, 10 nM or more, 20 nM or more, 30 nM or more, 40 nM or more, 50 nM or more, 60 nM or more, 70 nM or more, 80 nM or more, 90 nM or more, 100 nM or more, 200 nM or more, 300 nM or more, 400 nM or more, 500 nM or more, 600 nM or more, or 700 nM or more).

The EC₅₀ of the compound at ERβ can range from any of the minimum values described above to any of the maximum values described above. For example, the compounds disclosed herein can have an EC₅₀ of from 1 pM to 800 nM at ERβ (e.g., from 1 pM to 400 nM, from 400 nM to 800 nM, from 1 pM to 300 nM, from 1 pM to 200 nM, from 1 pM to 100 nM, from 1 pM to 50 nM, from 1 pM to 20 nM, from 1 pM to 10 nM, from 1 pM to 6 nM, from 1 pM to 5 nM, from 1 pM to 2 nM, from 1 pM to 1 nM, from 1 pM to 0.7 nM, from 1 pM to 0.5 nM, from 1 pM to 0.2 pM, or from 1 pM to 0.1 nM).

In some examples, the compounds disclosed herein are selective ERβ agonist. In some examples, the compound is a selective ERβ agonist in both a human model and a mouse model. In some examples, a selective ERβ agonist is a compound that has a lower EC₅₀ at ERβ than at estrogen receptor α (ERα). The selectivity of the compounds can, in some examples, be expressed as an ERβ-to-ERα agonist ratio, which is the EC₅₀ of the compound at ERα divided by the EC₅₀ of the compound at ERβ. In some examples, the compounds disclosed herein can have an ERβ-to-ERα agonist ratio of 8 or more (e.g., 10 or more, 20 or more, 30 or more, 40 or more, 50 or more, 60 or more, 70 or more, 80 or more, 90 or more, 100 or more, 150 or more, 200 or more, 250 or more, 300 or more, 350 or more, 400 or more, 450 or more, 500 or more, 600 or more, 700 or more, 800 or more, 900 or more, 1000 or more, 1100 or more, 1200 or more, 1300 or more, 1400 or more, 1500 or more, 2000 or more, 2500 or more). In some examples, the compound has an ERβ-to-ERα agonist ratio of 8 or more in both a human model and a mouse model. In some examples, the compound has an ERβ-to-ERα agonist ratio of 400 or more.

In some examples, the compounds can have an ERβ-to-ERα agonist ratio of 3000 or less (e.g., 2500 or less, 2000 or less, 1500 or less, 1400 or less, 1300 or less, 1200 or less, 1100 or less, 1000 or less, 900 or less, 800 or less, 700 or less, 600 or less, 500 or less, 450 or less, 400 or less, 350 or less, 300 or less, 250 or less, 200 or less, 150 or less, 100 or less, 90 or less, 80 or less, 70 or less, 60 or less, 50 or less, 40 or less, 30 or less, 20 or less, or 10 or less).

The ERβ-to-ERα agonist ratio of the compounds at ERβ can range from any of the minimum values described above to any of the maximum values described above. For example, the compounds can have an ERβ-to-ERα agonist ratio of from 8 to 3000 (e.g., from 8 to 1500, from 1500 to 3000, from 400 to 3000, from 500 to 3000, from 600 to 3000, from 700 to 3000, from 800 to 3000, from 900 to 3000, from 1000 to 3000, or from 2000 to 3000).

In some examples, the carborane cluster includes a heteroatom.

In some examples, the carborane cluster includes an isotopically labeled atom (i.e., a radiolabeled atom). In some examples, the isotopically labeled atom includes ¹⁰B. In some examples, the isotopically labeled atom includes a radiohalogen bound to the carborane cluster.

Also disclosed herein are pharmaceutical compositions comprising the compounds described herein, and a pharmaceutically acceptable excipient.

Also disclosed herein are pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Katchem (Prague, Czech Republic), Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical excipients disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

### Methods of Use

Also provided herein are methods of use of the compounds or compositions described herein. Also provided herein are methods for treating a disease or pathology in a subject in need thereof comprising administering to the subject a therapeutically effective amount of any of the compounds or compositions described herein.

Also provided herein are methods of evaluating the clinical efficacy of an ERβ agonis in a human patient. The methods can, for example, comprise administering the ERβ agonist to a non-human preclinical species model, wherein the ERβ agonist has an ERβ-to-ERα ratio of 8 or more in both a human model and the non-human preclinical species model. In some examples, the ERβ agonist comprises any of the compounds disclosed herein. In some examples, the preclinical species model comprises a mouse model. In some examples, the method is in vitro or in vivo.

Also provided herein are methods of treating, preventing, or ameliorating cancer in a subject, suppressing tumor growth in a subject, treating an inflammatory disease in a subject, treating a neurodegenerative disease in a subject, treating a psychotropic disorder in a subject, or a combination thereof, by administering to a subject a therapeutically effective amount of one or more of the compounds or compositions described herein, or a pharmaceutically acceptable salt thereof.

For example, also disclosed herein are methods of treating cancer in a subject. The methods include administering to the subject a therapeutically effective amount of any of the compounds or compositions disclosed herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma. In some examples, the cancer is selected from the group consisting of breast cancer, colorectal cancer, endometrial cancer, ovarian cancer, and prostate cancer.

The methods of treatment or prevention of cancer described herein can further include treatment with one or more additional agents (e.g., an anti-cancer agent or ionizing radiation). In some examples, the methods further comprise co-administering an anticancer agent to the subject. The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

For example, the compounds or compositions or pharmaceutically acceptable salts thereof as described herein can be combined into a pharmaceutical composition with an additional anti-cancer agent, such as 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also disclosed herein are methods of suppressing tumor growth in a subject. The methods comprise contacting at least a portion of the tumor with a therapeutically effective amount of any of the compounds or compositions disclosed herein, and optionally includes the step of irradiating at least a portion of the tumor with a therapeutically effective amount of ionizing radiation. As used herein, the term ionizing radiation refers to radiation comprising particles or photons that have sufficient energy or can produce sufficient energy via nuclear interactions to produce ionization. An example of ionizing radiation is x-radiation. A therapeutically effective amount of ionizing radiation refers to a dose of ionizing radiation that produces an increase in cell damage or death when administered in combination with the compounds described herein. The ionizing radiation can be delivered according to methods as known in the art, including administering radiolabeled compounds and radioisotopes.

Also disclosed herein are methods of treating an inflammatory disease in a subject. The methods comprise administering to the subject a therapeutically effective amount of any of the compounds or compositions disclosed herein. Inflammatory diseases include, but are not limited to, acne vulgaris, ankylosing spondylitis, asthma, autoimmune diseases, Celiac disease, chronic prostatitis, Crohn's disease, glomerulonephritis, hidradenitis suppurativa, inflammatory bowel diseases, pelvic inflammatory disease, psoriasis, reperfusion injury, rheumatoid arthritis, sarcoidosis, vasculitis, interstitial cystitis, type 1 hypersensitivities, systemic sclerosis, dermatomyositis, polymyositis, and inclusion body myositis. In some examples, the inflammatory disease is selected from the group consisting of arthritis and inflammatory bowel disease. In some examples, the methods further comprise co-administering an anti-inflammatory agent to the subject.

The methods of treatment of inflammatory diseases described herein can further include treatment with one or more additional agents (e.g., an anti-inflammatory agent). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

Also disclosed herein are methods of treating a neurodegenerative disease in a subject. The methods comprise administering to the subject a therapeutically effective amount of any of the compounds or compositions disclosed herein. Neurodegenerative diseases include, but are not limited to, Alzheimer's disease, amyotrophic lateral sclerosis (ALS), Alpers' disease, batten disease, Benson's syndrome, Cerebro-oculo-facio-skeletal (COFS) syndrome, corticobasal degeneration, Creutzfeldt-Jakob disease, dementias, Friedreich's ataxia, Gerstmann-Strussler-Scheinker disease, Huntington's disease, Lewy body syndrome, Leigh's disease, monomelic amyotrophy, motor neuron diseases, multiple system atrophy, opsoclonus myoclonus, progressive multifocal leukoencephalopathy, Parkinson's disease, Prion diseases, primary progressive aphasia, progressive supranuclear palsy, spinocerebellar ataxia, spinal muscular atrophy, kuru, and Shy-Drager syndrome.

Also disclosed herein are methods of treating a psychotropic disorder in a subject. The methods comprise administering to the subject a therapeutically effective amount of any of the compounds or compositions disclosed herein. Psychotropic disorders include, but are not limited to, attention deficit disorder (ADD), attention deficit hyperactive disorder (ADHD), anorexia nervosa, anxiety, dipolar disorder, bulimia, depression, insomnia, neuropathic pain, mania, obsessive compulsive disorder (OCD), panic disorder, premenstrual dysphoric disorder (PMDD), mood disorder, serotonin syndrome, schizophrenia, and seasonal affective disorder.

The compounds described herein can also be used to treat other ERβ-related (ERβ-mediated) diseases, including cardiovascular diseases (e.g., heart attack, heart failure, ischemic stroke, arrhythmia), benign prostatic hyperplasia, and osteoporosis.

Also disclosed herein are methods of imaging a cell or a population of cells expressing ERβ within or about a subject. The methods comprise administering to the subject an amount of any of the compounds or compositions disclosed herein; and detecting said compound or composition. The detecting can involve methods known in the art, for example, positron emission tomography (PET), single-photon emission computed tomography (SPECT), magnetic resonance imaging (MRI), X-ray, microscopy, computed tomography (CT). In some examples, the compound or composition can further comprise a detectable label, such as a radiolabel, fluorescent label, enzymatic label, and the like. In some examples, the detectable label can comprise a radiolabel, such as ¹⁰B. Such imaging methods can be used, for example, for assessing the extent of a disease and/or the target of a therapeutic agent.

In some examples, the cell or population of cells is indicative of cancer, an inflammatory disease, a neurodegenerative disease, a psychotropic disorder, or a combination thereof. In some examples, the cell or population of cells is indicative of cancer. In some examples, the cancer is selected from the group consisting of breast cancer, colorectal cancer, and prostate cancer. In some examples, the cell or population of cells is indicative of an inflammatory disease. In some examples, the inflammatory disease is selected from the group consisting of arthritis and inflammatory bowel disease.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of the disease or disorder), during early onset (e.g., upon initial signs and symptoms of the disease or disorder), or after an established development of the disease or disorder. Prophylactic administration can occur for several days to years prior to the manifestation of symptoms of a disease or disorder. Therapeutic treatment involves administering to a subject a therapeutically effective amount of the compounds and compositions or pharmaceutically acceptable salts thereof as described herein after the disease or disorder is diagnosed.

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that a therapeutically effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise any of the compounds disclosed herein in combination with a pharmaceutically acceptable excipient. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

A number of embodiments of the invention have been described. Nevertheless, it will be understood that various modifications may be made without departing from the spirit and scope of the invention. Accordingly, other embodiments are within the scope of the following claims.

### EXAMPLES

The following examples are set forth to illustrate the methods and results according to the disclosed subject matter. These examples are not intended to be inclusive of all aspects of the subject matter disclosed herein, but rather to illustrate representative methods and results. These examples are not intended to exclude equivalents and variations which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

### Example 1—the Elucidation of Species-Specific Receptor Pharmacology: A Case Study Using Subtype Selective Para- and Meta-Carborane Estrogen Receptor Agonists

Abstract. Estrogen receptors are essential pharmacological targets for treating hormonal disorders and estrogen-dependent malignancies. Selective activation of estrogen receptor (ER) R is hypothesized to provide therapeutic benefit with reduced risk of unwanted estrogenic side-effects associated with ERα activity. However, activating ERβ without activating a is challenging due to the high sequence and structural homology between the receptor subtypes. The impact of structural modifications to compound OSU-ERβ-12 on receptor subtype binding selectivity was assessed using cell-free binding assays. Functional selectivity was evaluated by transactivation in HEK-293 cells overexpressing human or murine estrogen receptors. In vivo selectivity was examined through the uterotrophic effects of the analogs after oral administration in estrogen-naïve female mice. Furthermore, the in vivo pharmacokinetics of the analogs following single dose IV and oral administration were evaluated. Regarding selectivity, a single compound exhibited greater functional selectivity than OSU-ERβ-12 for human ERβ. However, like others in the meta-carborane series, its poor in vivo pharmacokinetics limit its suitability for further development. Surprisingly, and at odds with their pharmacokinetic and in vitro human activity data, most analogs potently induced uterotrophic effects in estrogen-naïve female mice. Further investigation of activity in HEK293 cells expressing murine estrogen receptors revealed species-specific differences in the ER-subtype selectivity of these analogs. These findings highlight species-specific receptor pharmacology and the challenges it poses to characterizing developmental therapeutics in preclinical species.

This study investigates para- and meta-substituted carborane analogs targeting estrogen receptors, revealing the greater selectivity of carborane analogs for human ERβ compared to the mouse homolog. These findings shed light on the intricacies of using preclinical species in drug development to predict human pharmacology. The report also provides insights for the refinement and optimization of carborane analogs as potential therapeutic agents for estrogen-related disease states.

Introduction. Since the development of tamoxifen in the 1970s, selective estrogen receptor modulators (SERMS) have been used clinically for the management of breast cancer and as hormone replacement therapies for osteoporosis in postmenopausal women (An, 2016; Archer, 2011). SERMs are “selective” in that they have been shown to elicit ER agonist or antagonist activity depending on the tissue context. For example, in bone tissue, SERMs act as ER agonists to support bone density in postmenopausal women, but they can also act as antagonists in breast tissue making them an effective, widely prescribed, tool for the management of ER+ breast cancer (An, 2016; Jordan & Brodie, 2007; Swaby et al., 2007). However, first-generation SERMs such as tamoxifen have trophic effects on endometrial tissue, which may increase the risk of endometrial cancer (Archer, 2011). Many factors have been shown to contribute to the nature of SERM pharmacology, including the differential expression of estrogen receptors α and β in a given tissue, differences in their receptor conformation upon ligand binding, and differences in the expression and binding of transcriptional co-regulator proteins (Riggs & Hartmann, 2003).

Of the two subtypes of nuclear estrogen receptors, ERα is most often associated with the activating phenotype causing proliferative effects of estrogens, especially in breast and endometrial tissues (Riggs & Hartmann, 2003), which may increase the risk of cancers in these tissues (Heldring et al., 2007). ERα activation is also strongly associated with control of sex hormone biogenesis through negative feedback within the hypothalamic-pituitary-gonadal axis (Dupont et al., 2000) and hepatic expression of clotting factors (Cleuren et al., 2010). On the other hand, estrogen signaling ascribable to ERβ is less clear, but several mouse models of genetically ablated ERβ suggest a critical role for ERβ in immune and metabolic homeostasis (Hewitt & Korach, 2018; Warner et al., 2020; Zidon et al., 2020). When taken together, these findings support the development of ERβ-selective agonists to take advantage of estrogens' beneficial effects mediated by ERβ (Heldring et al., 2007; Paterni et al., 2014). By selectively targeting ERβ over ERα, beneficial estrogen pharmacology could be retained in tissues of the CNS, cardiovascular, immune, and skeletal systems while avoiding many of the negative estrogenic effects limiting broader use of estrogen therapy (Jordan, 2001; Mohler et al., 2010). This therapeutic premise has resulted in several ERβ drug development programs, with two ligands, ERB-041 (prinaberel, (Roman-Blas et al., 2010)) and LY500307 (erteberel, (Roehrborn et al., 2015)) reaching Phase II clinical trials.

A major challenge to the synthesis of selective ER agonists is the high sequence homology and similar structural conformation in the ligand-binding domains of the two receptor subtypes. In most ER crystal structures, only two amino acid residues differ in their interactions with bound ER ligands between the subtypes (Paterni et al., 2014). Despite this, ERβ-selective ligands reported thus far represent considerable structural diversity, including carbon boron clusters known as carboranes (Farzaneh & Zarghi, 2016; Ohta et al., 2017). Recently, significant selectivity for ERβ over ERα was reported within a series of para-carborane ER agonists (Sedlák et al., 2021). Within this series, the carborane core and the phenol moiety were important for ensuring ER activity while the elongation of the alkyl side chain reduced potency generally but increased ERβ selectivity to more than 200-fold. The lead compound from this series (OSU-ERβ-12) has shown promising efficacy in an array of different disease contexts including, ER+ breast cancer, ovarian cancer, chronic heart failure, neuroinflammation, and nonalcoholic steatohepatitis to name a few (Banerjee et al., 2022; Datta et al., 2022; Grant et al., 2022; Helms et al., 2020; Kumar et al., 2023; Rosenzweig et al., 2022). Importantly, confirmed ERβ selective doses were capable of eliciting therapeutic responses.

One critical aspect of drug development is confirming the pharmacological relevance of preclinical species when considering model systems for both efficacy and toxicity. A great deal of attention is paid to identifying species differences in drug metabolism and pharmacokinetics to ensure IND enabling toxicology studies are performed in non-human species most closely reflecting the likely disposition of investigational agents in humans (Davies et al., 2020; Namdari et al., 2021; Thakur et al., 2024). Though just as important, rigorous efforts to confirm human analogous target receptor pharmacology in pre-clinical model species are seldom reported. When species differences are taken into account, comparisons are usually limited to in vitro, reconstituted assay systems (Asnake et al., 2019; Bingham et al., 2007) owing to the complexity of modelling most receptor pharmacology in vivo. In this work, the in vivo estrogen pharmacology of a series of synthesized para- and meta-carborane ERβ selective agonists were characterize in an effort to improve on the ERβ-directed potency and selectivity of OSU-ERβ-12. It is shown that OSU-ERβ-12, in addition to several other carborane subtype-selective ER agonists, has exceptional murine pharmacokinetics consistent with potential therapeutics. It is also demonstrated that in vivo murine estrogen pharmacology diverged from what human in vitro ER activity assays would predict. A re-evaluation of the analogs' activity in murine in vitro assay systems resolved the unexpected in vivo murine pharmacology data providing evidence of the importance of species-specific receptor pharmacology and highlighting the complexities of therapeutic estrogen drug development.

### Materials and Methods

¹H-NMR spectra were recorded at The Ohio State University College of Pharmacy using a Bruker AV300NMR, Bruker AVIII400HD NMR spectrometer or a Bruker DRX400 NMR spectrometer. Chemical shifts (6) are reported in ppm from chemical reference shifts for internal deuterated chloroform (CDCl₃) set to 7.26 ppm or deuterated DMSO (DMSO-d₆) set to 2.50 ppm. Coupling constants are reported in Hz. Mass spectra were obtained from the Ohio State University Comprehensive Cancer Center using an Advion Expression® Model S Compact Mass Spectrometer equipped with an APCI source and TLC plate Express™. For carborane-containing compounds, the found mass corresponding to the most intense peak of the theoretical isotopic pattern was reported. Measured patterns agreed with calculated patterns. Unless otherwise noted, all reactions were carried out under argon atmosphere using commercially available reagents and solvents. Chromatographic purification was performed using a Teledyne Isco CombiFlash® Rf⁺ lumen equipped with silica gel cartridges.

Commercially Available Chemicals and Reagents. Following previously published protocols (Sedlák et al., 2021), OSU-ERβ-12 was synthesized by the Medicinal Chemistry Shared Resource at The Ohio State University Comprehensive Cancer Center. Human ERα and ERβ expression vectors (pcDNA3-hERα and pcDNA3-hERβ, respectively), as well as the luciferase reporter vector (pGL4.26-3×ERE) were kindly provided by Dr. David Sedlák (CZ-OPENSCREEN; Prague, Czech Republic). For murine ERα and Erp expression vectors (pcDNA3-mERα and pcDNA3-mERβ, respectively), canonical sequences were synthesized and cloned between NheI and EcoRI sites of pcDNA3.1+(custom service of Bio Basic Inc., Markham, ON, Canada).

Chemical Synthesis. Synthesis of compounds 1, 4, and OSU-ERβ-12, was carried out according to the procedure outlined in (Sedlák et al., 2021). Compounds 2, 3 and 5, were synthesized using General Procedure A (Scheme 1); Compounds 7, 8, and 10 were synthesized using General Procedure B (Scheme 2); compounds 9 and 11 were synthesized according to Procedures for synthesis of compound 9 and 11 (Scheme 2), respectively. Compound 6 was synthesized using Procedure for Synthesis of compound 6 (Scheme 3).

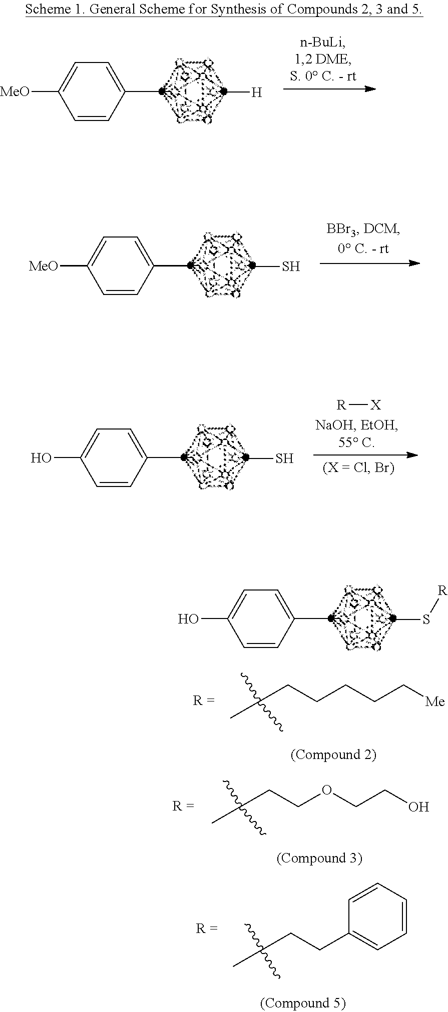

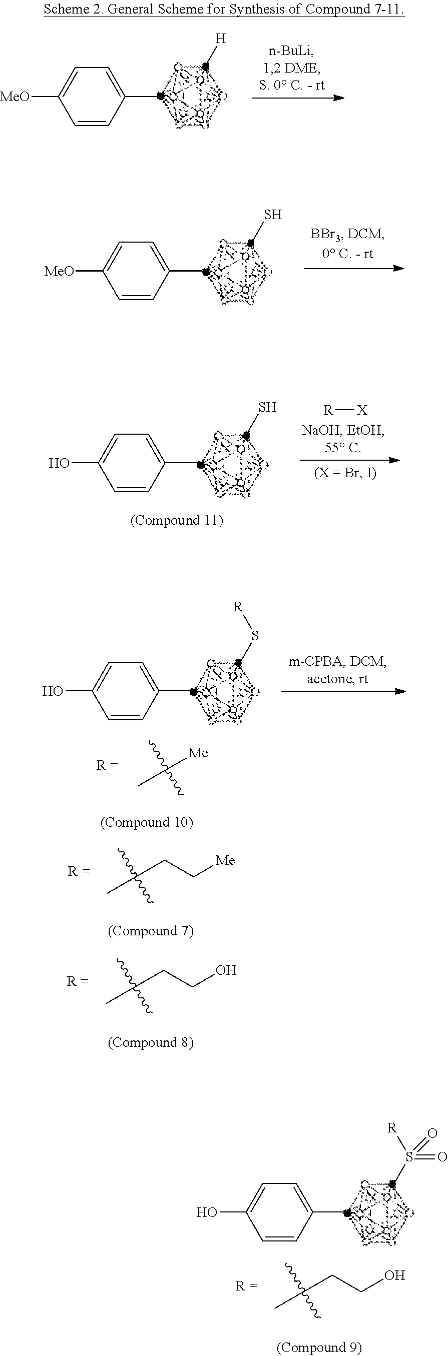

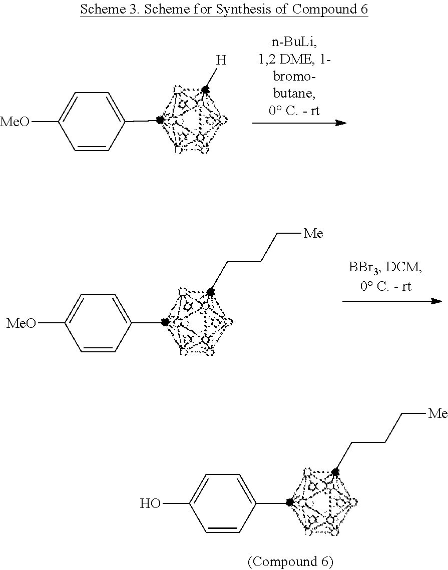

### General Procedure A for Synthesis of Compounds 2, 3 and 5:

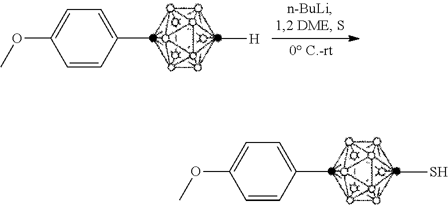

Step 1. 1-mercapto-12-(4-methoxyphenyl)-1,12-dicarba-closododecaborane. To a solution of 1-(4-methoxyphenyl)-1,12-dicarba-closo-dodecaborane (which was prepared in a manner similar to Endo Y et al. Chemistry & Biology, 2001, 8, 341-355) (5 g, 1.0 eq., 19.97 mmol) in 1,2-dimethoxyethane (166 ml) was added dropwise an n-butyllithium solution (2.5M in hexanes, 9.6 mL, 1.2 eq., 24 mmol) at 0° C. under argon. The mixture was stirred at room temperature for 1 hour. Elemental sulfur (801 mg, 1.25 eq., 24.97 mmol) was added portion-wise at room temperature. The mixture was stirred at room temperature for 10 min, quenched with H₂O, and acidified to pH 2 with 2N HCl. Ethyl acetate was added and the biphasic mixture was filtered through cotton and rinsed with additional ethyl acetate to remove the insoluble material. The organic layer was separated, and the aqueous layer was extracted two additional times with ethyl acetate. The organic layer was washed with brine, dried over Na₂SO₄, filtered, and concentrated in vacuo. The resulting residue was purified using Combiflash (SiO₂, RediSepRf 40 g column, gradient: DCM/Hex) to yield 2.93 g (52%) of a white powder. ¹H NMR (300 MHz, CDCl₃): δ 7.05 (app d, J=9.0 Hz, 2H), 6.67 (app d, J=9.0 Hz, 2H), 3.73 (s, 3H), 3.16 (br s, 1H), 1.40-3.60 (m, 10H). MS (APCI) m/z: [M-H]⁻ calcd. for C₉H₁₇B₁₀OS, 281.2; found 281.0.

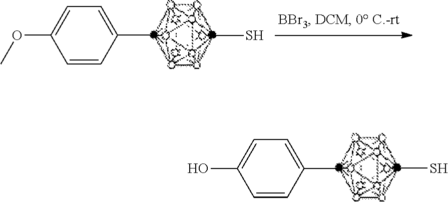

Step 2. 1-mercapto-12-(4-hydroxyphenyl)-1,12-dicarba-closododecaborane: To a solution of 1-mercapto-12-(4-methoxyphenyl)-1,12-dicarba-closododecaborane (1.18 g, 4.18 mmol) in dichloromethane (21 ml) was added dropwise a boron tribromide solution (1M in DCM, 12.5 mL, 3.0 eq, 12.5 mmol) at 0° C. under argon. The mixture was stirred at room temperature for 1.5 h, cooled to 0° C., quenched by slow addition of H₂O, and extracted 3× with dichloromethane. The organic layer was separated, washed with brine, dried over Na₂SO₄, filtered, and concentrated in vacuo. The resulting residue was purified using Combiflash (SiO₂, RediSepRf 24 g column, gradient: 0-20% EtOAc/Hex) to yield 1.01 g (90%) of a white powder. (A small aliquot was recrystallized from ethyl acetate in hexanes for an analytically pure sample). ¹H NMR (400 MHz, CDCl₃): δ 7.01 (app d, J=8.9 Hz, 2H), 6.60 (app d, J=8.9 Hz, 2H), 4.67 (br s, 1H), 3.16 (br s, 1H), 1.72-3.42 (m, 10H). MS (APCI) m/z: [M-H]⁻ calcd. for C₈H₁₅B₁₀OS, 267.2; found, 267.0.

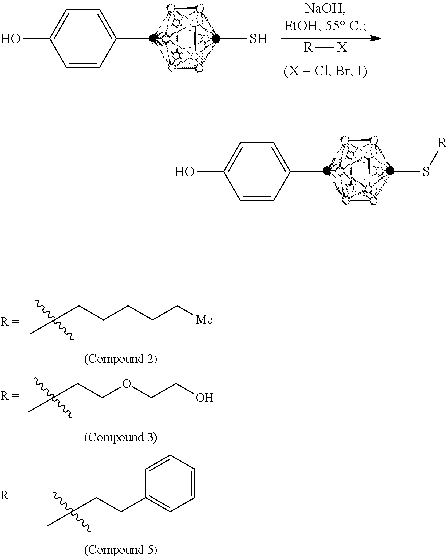

Step 3. To a solution of 1-mercapto-12-(4-hydroxyphenyl)-1,12-dicarba-closododecaborane (1.0 equivalents) in ethanol (0.042 M) was added NaOH (2.0-2.1 equivalents), the reaction mixture was stirred at 55° C. for 15 minutes before iodo/bromo/chloro alkane (1.0-1.25 equivalents) was added. Note: in the case of chloroalkanes, a catalytic amount of sodium iodide (0.02 equivalents) was added to the reaction mixture. The final reaction mixture was stirred at 55° C. overnight, cooled to room temperature and adjusted to pH 1 to 3 with 2N HCl. Ethanol was removed using a rotary evaporator and the residue was partitioned between ethyl acetate, and water. The aqueous layer was extracted 3× with ethyl acetate, and the combined organic layers were washed with saturated aqueous sodium bicarbonate, washed with 1000 aqueous sodium thiosulfate solution, and washed with brine. The organic layer was dried over Na₂SO₄, concentrated in vacuo and the residue was purified by CombiFlash Teledyne Isco (RediSepRf column) to afford product.

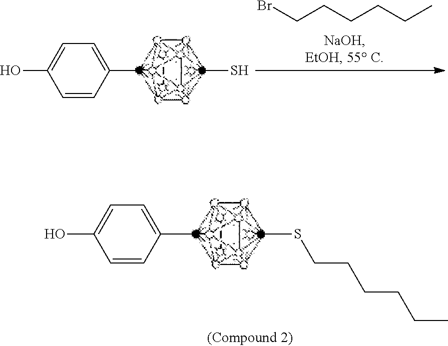

1-(4-hydroxyphenyl)-12-(hexylthio)-1,12-dicarba-closododecaborane (Compound 2). Prepared according to general procedure A using 1-mercapto-12-(4-hydroxyphenyl)-1,12-dicarba-closododecaborane (400 mg, 1.0 eq, 1.49 mmol), sodium hydroxide (125 mg, 2.1 eq, 3.13 mmol), and 1-bromohexane (271 mg, 1.1 eq, 1.64 mmol). Chromatography conditions: Gradient of ethyl acetate in hexanes (0-10%). Yield: 429 mg, white powder. ¹H NMR (400 MHz, CDCl₃): δ 7.04 (app d, J=8.9 Hz, 2H), 6.60 (app d, J=8.9 Hz, 2H), 4.74 (br s, 1H), 2.58 (t, J=7.3 Hz, 2H), 1.66-3.40 (m, 10H), 1.40-1.52 (m, 2H), 1.15-1.36 (m, 6H), 0.87 (t, 3H). MS (APCI) m/z: [M-H]⁻ calcd. for C₁₄H₂₇B₁₀OS, 351.3; found 351.0.

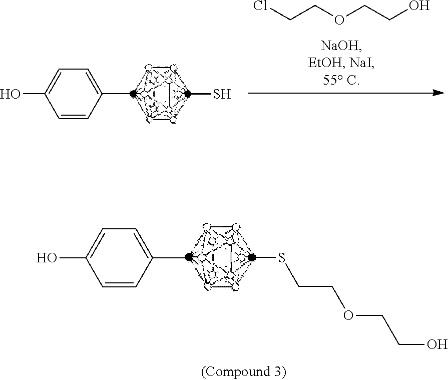

1-(4-hydroxyphenyl)-12-((2-(2-hydroxyethoxy)ethyl)thio)-1,12-dicarbaclosododecaborane (Compound 3). Prepared according to general procedure A using 1-mercapto-12-(4-hydroxyphenyl)-1,12-dicarba-closododecaborane (600 mg, 1.0 eq., 2.24 mmol), sodium hydroxide (179 mg, 2.0 eq, 4.47 mmol), 2-(2-chloroethoxy)ethanol (306 mg, 1.1 equivalents, 2.46 mmol), and sodium iodide (0.02 equivalents). Chromatography conditions: Gradient of ethyl acetate in hexanes. Yield: 545 mg, white crystalline powder. ¹H NMR (400 MHz, DMSO-d₆): δ 9.70 (s, 1H), 6.96 (app d, J=8.9 Hz, 2H), 6.59 (app d, J=8.9 Hz, 2H), 4.58 (br t, J=5.2 Hz, 1H), 3.41-3.50 (m, 4H), 3.32-3.39 (m, 2H), 2.80 (t, J=6.4 Hz, 2H), 1.60-3.54 (m, 10H). MS (APCI) m/z: [M-H]⁻ calcd. for C₁₂H₂₃B₁₀O₃S, 355.2; found, 355.0.

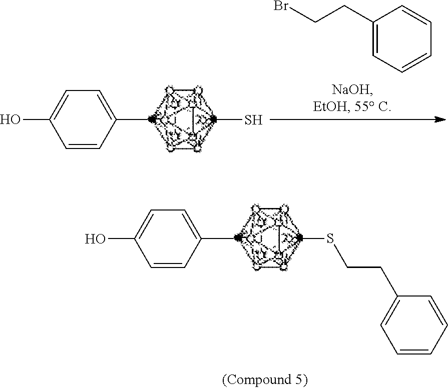

1-(4-hydroxyphenyl)-12-(phenethylthio)-1,12-dicarba-closododecaborane (Compound 5). Prepared according to general procedure A using 1-mercapto-12-(4-hydroxyphenyl)-1,12-dicarba-closododecaborane (400 mg, 1.0 eq., 1.49 mmol), sodium hydroxide (119 mg, 2.0 eq., 2.98 mmol), and (2-bromoethyl)benzene (303 mg, 1.1 eq., 1.64 mmol). Chromatography conditions: Gradient of ethyl acetate in hexanes. Yield: 320 mg, white powder. ¹H NMR (400 MHz, CDCl₃): δ 7.19-7.33 (m, 3H), 7.12-7.18 (m, 2H), 7.04 (app d, J=8.9 Hz, 2H), 6.60 (app d, J=8.9 Hz, 2H), 4.69 (br s, 1H), 2.80-2.86 (m, 2H), 2.72-2.79 (m, 2H), 1.70-3.40 (m, 10H). MS (APCI) m/z: [M-H]⁻ calcd. for C₁₆H₂₃B₁₀OS, 371.2; found, 371.0.

### Procedure for Synthesis of Compound 11:

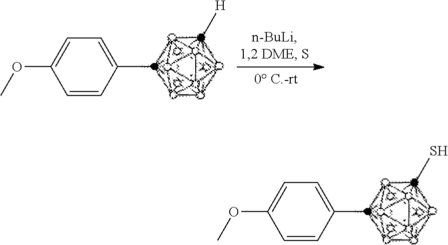

Step 1. 1-mercapto-7-(4-methoxyphenyl)-1,7-dicarba-closododecaborane. A solution of 1-(4-methoxyphenyl)-1,7-dicarba-closo-dodecaborane (which was prepared in a manner similar to Li et al. Journal of Organometallic Chemistry 2015, 798, 189-195) (1 g, 1.0 eq., 3.99 mmol) in 1,2 dimethoxyethane (33 ml) was vigorously degassed with argon for 5 mins and an n-BuLi solution (2.5 M in hexanes, 1.92 ml, 1.2 eq., 4.8 mmol) was added thereafter at 0° C. under argon. The mixture was stirred at room temperature for 1 hour followed by portion-wise addition of elemental sulfur (160 mg, 1.25 eq., 4.99 mmol) at room temperature. The mixture was stirred at room temperature for 15 min, quenched with H₂O (degassed with argon), and acidified to pH 2 with 2N HCl (degassed with argon). The reaction mixture was extracted 2× with ethyl acetate. The organic layer was washed with brine, dried over Na₂SO₄, filtered, and concentrated in vacuo to remove most of the dimethoxyethane. The resulting residue was re-suspended in hexanes, filtered through a small plug of cotton, and 10% aqueous KOH solution was added. The aqueous layer was washed 3× with hexanes and acidified to pH 2 with 2N HCl at which point the desired product formed a precipitate. The resulting product was filtered on a Buchner funnel under vacuum, washed with H₂O, and dried under vacuum at 70° C. to yield 0.69 g (61%) of an off-white powder. ¹H NMR (400 MHz, CDCl₃): δ 7.32 (app d, J=9.0 Hz, 2H), 6.77 (app d, J=9.0 Hz, 2H), 3.78 (s, 3H), 3.44 (br s, 1H), 1.46-4.04 (m, 10H). MS (APCI) m/z: [M-H]⁻ calcd. for C₉H₁₇B₁₀OS, 281.2; found, 281.0.

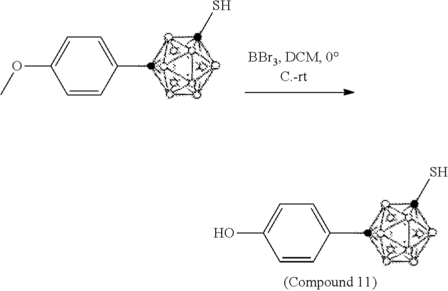

Step 2. 1-mercapto-7-(4-hydroxyphenyl)-1,7-dicarba-closododecaborane (Compound 11). To a solution of 1-mercapto-7-(4-methoxyphenyl)-1,7-dicarba-closododecaborane (650 mg, 1.0 eq., 2.30 mmol) in dichloromethane (11.5 ml) was added dropwise a boron tribromide solution (1M in DCM, 6.9 mL, 3.0 eq., 6.9 mmol) at 0° C. under argon. The mixture was stirred at room temperature for 3 h, cooled to 0° C., quenched by slow addition of H₂O, and extracted 3× with dichloromethane. The organic layer was separated, washed with brine, dried over Na₂SO₄, filtered, and concentrated in vacuo. The resulting residue was purified using Combiflash (SiO₂, RediSepRf 12 g column, gradient: EtOAc/Hex) to yield 536 mg (87%) of an off-white powder. ¹H NMR (400 MHz, CDCl₃): δ 7.28 (app d, J=8.9 Hz, 2H), 6.70 (app d, J=8.9 Hz, 2H), 4.77 (br s, 1H), 3.44 (br s, 1H), 1.50-4.00 (m, 10H). MS (APCI) m/z: [M-H]⁻ calcd. for C₈H₁₅B₁₀OS, 267.2; found, 267.0.

### General Procedure B for Synthesis of Compounds 7, 8, and 10:

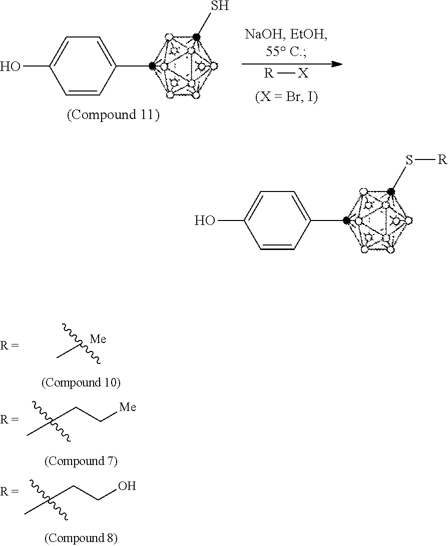

To a solution of 1-mercapto-7-(4-hydroxyphenyl)-1,7-dicarba-closododecaborane (Compound 11) (1.0 equivalents) in ethanol (0.042 M) was added NaOH (2.0 equivalents), the reaction mixture was stirred at 55° C. for 15 minutes before the corresponding iodo/bromo-alkane (1.0-1.25 equivalents) was added. (In select cases, catalytic sodium iodide (0.02 eq) was added.) The final reaction mixture was stirred at 55° C. for 6 hours to overnight, cooled to room temperature and adjusted to pH 1 to 3 with 2N HCl. Ethanol was removed using a rotary evaporator and the residue was partitioned between ethyl acetate, and water. The aqueous layer was extracted 3× with ethyl acetate, and the combined organic layers were washed with saturated aqueous sodium bicarbonate, washed with 10% aqueous sodium thiosulfate solution, and washed with brine. The organic layer was dried over Na₂SO₄ concentrated in vacuo and the residue was purified by CombiFlash Teledyne Isco (RediSepRf column) to afford products.

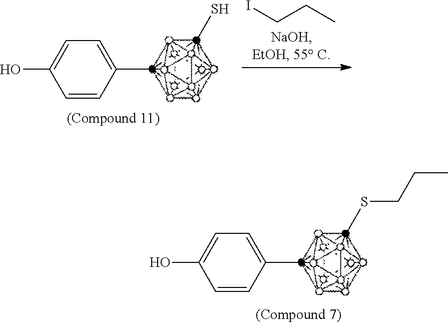

1-(4-hydroxyphenyl)-7-(propylthio)-1,7-dicarba-closododecaborane (Compound 7). Prepared according to general procedure B using 1-mercapto-7-(4-hydroxyphenyl)-1,7-dicarba-closododecaborane (Compound 11) (400 mg, 1.0 eq., 1.49 mmol), and 1-iodopropane (279 mg, 1.1 eq., 1.64 mmol). Time: overnight. Chromatography conditions: Gradient of ethyl acetate in hexanes. Yield: 398 mg, white waxy solid. ¹H NMR (400 MHz, CDCl₃): δ 7.28 (app d, J=8.8 Hz, 2H), 6.70 (app d, J=8.8 Hz, 2H), 4.76 (br s, 1H), 2.74 (t, J=7.4 Hz, 2H), 1.58 (sext, J=7.4 Hz, 2H), 1.40-4.00 (m, 10H), 0.97 (t, J=7.4 Hz, 3H). MS (APCI) m/z: [M-H]⁻ calcd. for C₁₁H₂₁B₁₀OS, 309.2; found, 309.0.

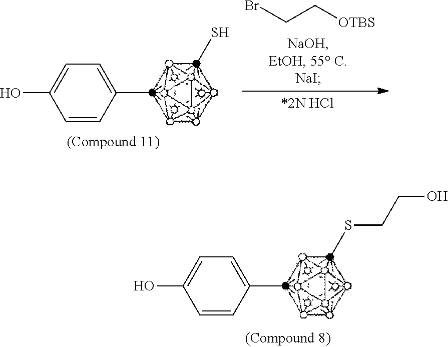

1-(4-hydroxyphenyl)-7-((2-hydroxyethyl)thio)-1,7-dicarba-closododecaborane (Compound 8). Prepared according to general procedure B using 1-mercapto-7-(4-hydroxyphenyl)-1,7-dicarba-closododecaborane (Compound 11) (385 mg, 1.0 eq., 1.44 mmol), (2-bromoethoxy)(tert-butyl)dimethylsilane (378 mg, 1.1 eq., 1.58 mmol) and catalytic sodium iodide. Time: 6 hours. (Additionally, after following the workup procedure, 7 mL of 2N HCl was added to ethanol and concentrated in vacuo at 50° C.) Chromatography conditions: Gradient of ethyl acetate in hexanes. Yield: 370 mg, white crystalline powder. ¹H NMR (400 MHz, DMSO-d₆): δ 9.81 (br s, 1H), 7.23 (app d, J=8.9 Hz, 2H), 6.69 (app d, J=8.9 Hz, 2H), 5.00 (br t, J=5.5 Hz, 1H), 3.50-3.58 (m, 2H), 2.92 (t, J=6.5 Hz, 2H), 1.02-3.80 (m, 10H). MS (APCI) m/z: [M-H]⁻ calcd. for C₁₀H₁₉B₁₀O₂S, 311.2; found, 311.0.

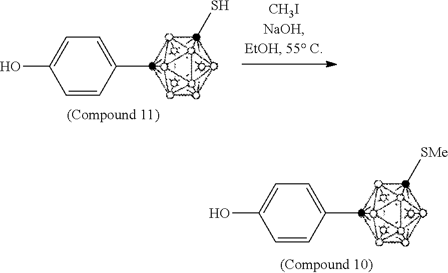

1-(4-hydroxyphenyl)-7-(methylthio)-1,7-dicarba-closododecaborane (Compound 10). Prepared according to general procedure B using 1-mercapto-7-(4-hydroxyphenyl)-1,7-dicarba-closododecaborane (Compound 11) (400 mg, 1.0 eq., 1.49 mmol), and iodomethane (233 mg, 1.1 eq., 1.64 mmol). Time: overnight. Chromatography conditions: Gradient of ethyl acetate in hexanes. Yield: 306 mg, white crystalline fluffy powder (recrystallization from hexanes). ¹H NMR (400 MHz, CDCl₃): δ 7.28 (app d, J=8.8 Hz, 2H), 6.70 (app d, J=8.8 Hz, 2H), 4.73 (br s, 1H), 2.30 (s, 3H), 1.40-4.00 (m, 10H). MS (APCI) m/z: [M-H]⁻ calcd. for C₉H₁₇B₁₀OS, 281.2; found, 281.0.

### Procedure for Synthesis of Compound 9:

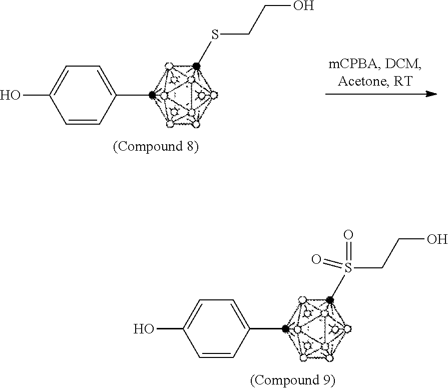

1-(4-hydroxyphenyl)-7-((2-hydroxyethyl)sulfonyl)-1,7-dicarba-closododecaborane (Compound 9). To a solution of 1-(4-hydroxyphenyl)-7-((2-hydroxyethyl)thio)-1,7-dicarba-closododecaborane (Compound 8) (350 mg, 1.0 eq., 1.12 mmol) in dichloromethane (22.4 mL, 0.05M) and acetone (5.33 mL, 0.21M) was added 3-chloroperoxybenzoic acid (77%, 527 mg, 2.1 eq., 2.35 mmol). The reaction mixture was stirred overnight at room temperature. The reaction mixture was carefully concentrated under a gentle stream of argon and quenched with 10% aqueous Na₂S₂O₃. The aqueous layer was extracted 3× with ethyl acetate, and the combined organic layers were washed with sat. aqueous NaHCO₃ (2×), washed with brine, dried over Na₂SO₄, and concentrated in vacuo. The residue was purified using Combiflash (SiO₂, RediSepRf 12 g column, gradient: EtOAc/Hex) to afford 329 mg of product as a white crystalline solid. ¹H NMR (400 MHz, DMSO-d₆): δ 9.88 (br s, 1H), 7.27 (app d, J=8.8 Hz, 2H), 6.71 (app d, J=8.8 Hz, 2H), 5.22 (br t, J=5.6 Hz, 1H), 3.84-3.90 (m, 2H), 3.63 (t, J=6.0 Hz, 2H), 1.30-3.80 (m, 10H). MS (APCI) m/z: [M-H]⁻ calcd. for C₁₀H₁₉B₁₀O₄S, 343.2; found, 343.0.

### Procedure for Synthesis of Compound 6:

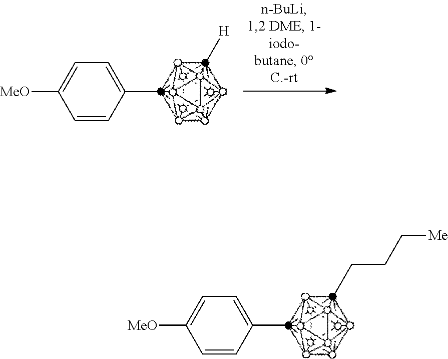

Step 1. 1-(4-methoxyphenyl)-7-(butyl)-1,7-dicarba-closododecaborane. A solution of 1-(4-methoxyphenyl)-1,7-dicarba-closo-dodecaborane (1.00 g, 3.99 mmol) in 1,2-dimethoxyethane (40.0 mL) was cooled to 0° C. and n-butyllithium (2.5M in hexanes, 2.00 mL, 1.3 eq., 4.99 mmol) was added slowly. The reaction mixture was warmed to rt and stirred for 1 hour. Iodobutane (681 μL, 1.5 eq., 5.99 mmol) was added to the reaction mixture and the reaction mixture was stirred overnight at rt. The reaction mixture was carefully quenched with water at 0° C. and poured into 2N HCl. The aqueous layer was extracted with ethyl acetate (3×). The combined organic layers were washed with saturated aqueous sodium bicarbonate solution, washed with 10% aqueous sodium thiosulfate, washed with brine, dried over sodium sulfate, filtered, and concentrated in vacuo. Using the combiflash, the residue was dry loaded onto silica gel using DCM and loaded onto a 12 g column eluting with a gradient of ethyl acetate in hexanes. Fractions containing desired product were concentrated in vacuo to yield 1.07 g of product as a clear colorless oil. ¹H NMR (400 MHz, CDCl₃): δ 7.33 (app d, J=9.0 Hz, 2H), 6.76 (app d, J=9.0 Hz, 2H), 3.78 (s, 3H), 1.94-2.00 (m, 2H), 1.40-4.00 (m, 10H), 1.32-1.42 (m, 2H), 1.19-1.30 (m, 2H), 0.88 (t, J=7.2 Hz, 3H).

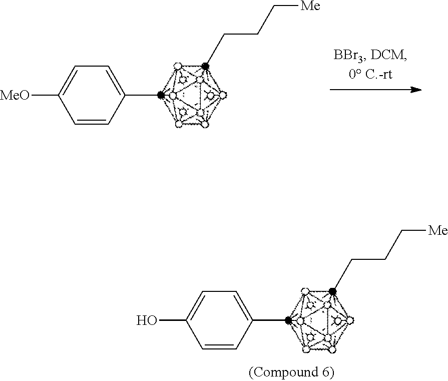

Step 2. 1-(4-hydroxyphenyl)-7-(butyl)-1,7-dicarba-closododecaborane (Compound 6). A solution of 1-(4-methoxyphenyl)-7-(butyl)-1,7-dicarba-closododecaborane (800 mg, 1.0 eq., 2.61 mmol) in DCM (65.0 mL) was cooled to 0° C. and a solution of boron tribromide (1M in DCM, 7.83 mL, 3.0 eq., 7.83 mmol) was added dropwise. The reaction mixture was stirred overnight at room temperature. The reaction mixture was quenched with 2N HCl, and the aqueous layer was extracted with DCM (3×). The combined organic layers were washed with brine, dried over sodium sulfate, filtered, and concentrated in vacuo. The residue was purified using Combiflash (SiO₂, RediSepRf 12 g column, gradient: EtOAc/Hex). Fractions containing desired product were concentrated in vacuo to afford 720 mg of product as a white solid. ¹H NMR (400 MHz, CDCl₃): δ 7.28 (app d, J=8.9 Hz, 2H), 6.69 (app d, J=8.9 Hz, 2H), 4.84 (br s, 1H), 1.94-2.00 (m, 2H), 1.40-4.00 (m, 10H), 1.32-1.42 (m, 2H), 1.19-1.30 (m, 2H), 0.88 (t, J=7.3 Hz, 3H). MS (APCI) m/z: [M-H]⁻ calcd. for C₁₂H₂₃B₁₀O, 291.3; found, 291.1.

Estrogen Receptor Transcriptional Activation Assays. HEK-293 cells (CRL-1573, ATCC, Manassas, VA, USA) were seeded on 100 mm dishes and grown in DMEM/10% FBS to ˜80% confluency. One day before transfection, the growth medium was replaced by phenol red-free DMEM/10% charcoal-stripped FBS (CS-FBS) without Penicillin-Streptomycin. DNA/Lipofectamine 2000 complexes were made by mixing 6 g of human (pcDNA3-hERα, or pcDNA3-hERβ) or murine (pcDNA3-mERα or pcDNA3-mERβ) ER expression vectors with 1.5 mg of pGL4.26-3×ERE in 1.5 mL of OPTI-MEM and adding to 60 μL Lipofectamine 2000 (Invitrogen) diluted in 1.5 mL of OPTI-MEM. The transfection procedure was performed as recommended by the manufacturer. Four to six hours later, cells were detached with 0.25% phenol red-free trypsin, resuspended in phenol red-free DMEM/10% CS-FBS to a density of 4×105 cells/mL, and aliquoted 90 μL/well on 96-well plates. Stock solutions of compounds were made in DMSO, diluted to 300 μM in phenol red-free DMEM/10% CS-FBS and further serial semi-log dilutions were prepared. Cells were stimulated with drugs by adding 10 μL of 10-fold concentrated compound to each well to achieve the final concentrations as indicated. Following an 18-hour incubation at 37° C., the culture medium was aspirated, cells were lysed in 70 μL 1× Passive Lysis Buffer (Promega, Madison, WI, USA) for 20 minutes at room temperature and 20 L of lysate was used to measure luciferase activity using Luciferase Assay System (Promega). Presented transactivation data represent biological triplicates gathered from three independent transfections performed on cells of three different passages.

Estrogen Receptor Radio Ligand Binding Assays. ER binding assays were performed by Eurofins Cerep (Celle-Levéscault, France) as previously reported (Datta et al., 2022). Briefly, ERα and ERβ were obtained from recombinant insect Sf9 cells overexpressing either human receptor. [³H] Estradiol (0.5 nM) and Diethylstilbestrol (1 μM) were used as radio-ligand and ER binding ligand positive controls, respectively. To determine the IC₅₀, each analog was tested at several concentrations depending on the receptor being challenged. The test concentrations ranged from 1 pM-10 μM for ERβ, and 10 pM-100 μM for ERα and were performed in duplicate. The IC₅₀ values were determined by non-linear least squares regression analysis using MathIQ™ (ID Business Solutions Ltd., UK). Ki values of the tested compounds were obtained by incorporating the IC₅₀, concentration of radioligand, and previously determined values for K_{D} of the ligand into the Cheng and Prusoff equation (Cheng & Prusoff, 1973). The Hill coefficients of the competition curves were calculated using MathIQ™.

Murine Pharmacokinetic Studies. Mouse pharmacokinetic studies were performed at Charles River Laboratories (Worcester, MA). Male C57/BL6 mice (approximately 30 grams, n=3 per group) were administered single doses of OSU-ERβ-12 or analogs according to the design in Table 1. Body weights measured before dose administration were used to calculate the volume (mL/kg) of the administered dose. Blood samples were collected primarily via the tail vein according to the sampling scheme in Table 2. Samples were collected in tubes with appropriate anticoagulant (K₂EDTA) which were stored on ice before centrifugation. Plasma samples were stored at −80° C. until transferred for LC-MS analysis.

TABLE 1
Agent administration information for mouse PK studies.

| | | Dose | Dose | Dose | | |
|---|---|---|---|---|---|---|
| | | Level | Concentration | Volume | | |
| Group | Analogue | (mg/kg) | (mg/mL) | (ml/kg) | Vehicle^{a} | Route |
| 1 | Compound 3 | 1 | 0.5 | 2 | 20% HPBCD in water | IV |
| 2 | Compound 3 | 3 | 0.5 | 6 | 20% HPBCD in water | PO |
| 3 | Compound 8 | 10 | 5 | 2 | 20% HPBCD in water | IV |
| 4 | Compound 8 | 100 | 5 | 20 | 20% HPBCD in water | PV |
| 5 | Compound 2 | 1 | 0.2 | 5 | 20% HPBCD in water | IV |
| 6 | Compound 2 | 10 | 1 | 10 | 20% HPBCD in water | PO |
| 7 | Compound 5 | 1 | 0.2 | 5 | 40% HPBCD in water | IV |
| 8 | Compound 5 | 10 | 1 | 10 | 40% HPBCD in water | PO |
| 9 | Compound 1 | 1 | 0.2 | 5 | 20% HPBCD in water | IV |
| 10 | Compound 1 | 10 | 1 | 10 | 20% HPBCD in water | PO |
| 11 | Compound 4 | 1 | 0.2 | 5 | 40% HPBCD in water | IV |
| 12 | Compound 4 | 10 | 1 | 10 | 40% HPBCD in water | PO |
| 13 | Compound 6 | 1 | 0.2 | 5 | 20% HPBCD in water | IV |
| 14 | Compound 6 | 10 | 1 | 10 | 20% HPBCD in water | PO |
| 15 | Compound 9 | 1 | 0.2 | 5 | 20% HPBCD in water | IV |
| 16 | Compound 9 | 10 | 1 | 10 | 20% HPBCD in water | PO |
| 17 | Compound 7 | 1 | 0.2 | 5 | 20% HPBCD in water | IV |
| 18 | Compound 7 | 10 | 1 | 10 | 20% HPBCD in water | PO |
| 19 | Compound 11 | 1 | 0.2 | 5 | 20% HPBCD in water | IV |
| 20 | Compound 11 | 10 | 1 | 10 | 20% HPBCD in water | PO |
| 21 | Compound 10 | 1 | 0.2 | 5 | 20% HPBCD in water | IV |
| 22 | Compound 10 | 10 | 1 | 10 | 20% HPBCD in water | PO |
| 23 | OSU-ERβ-12 | 10 | 1 | 10 | 5% DMSO/5% Tween20/water | IV |
| 24 | OSU-ERβ-12 | 10 | 5 | 2 | 5% DMSO/5% Tween20/water | PO |

^{a}HPBCD vehicles were prepared in water w/w; DMSO/Tween20 vehicles were prepared in water v/v/v.

TABLE 2
Serial blood collection schemes for mouse PK studies.

| | | Blood Collection Timepoints |
|---|---|---|
| Group | Route | (hours post-dose)^{a} |
| 1, 3, 15, 17, 19, and 21 | IV | 0.083, 0.25, 0.5, 1, 2, 6, 24, 48 |
| 2, 4, 16, 18, 20, and 22 | PO | 0.25, 0.5, 1, 2, 4, 8, 24, 48 |
| 5, 7, 9, 11, and 13 | IV | 0.083, 0.25, 0.5, 1, 2, 8, 24, 48 |
| 6, 8, 10, 12, and 14 | PO | 0.25, 0.5, 1, 2, 4, 6, 24, 48 |
| 23 | PO | 0.25, 0.5, 1, 2, 4, 8 |
| 24 | IV | 0.083, 0.25, 0.5, 1, 2, 6, 24 |

^{a}Anticoagulant: K₂EDTA; Volume collected per timepoint: ~35 μL

Liquid Chromatography-Mass Spectrometry Analyses of ER Subtype Selective Agonists in Mouse Plasma. LC-MS bioanalyses were performed at Charles River Labs in accordance with non-GLP RGA2 criteria (QCs within +/−20% or 25% at the LLOQ). The details of the analysis for each analog are summarized in Table 3.

TABLE 3
LC-MS analysis of SERMS in mouse plasma

| | | | | | Standard |
|---|---|---|---|---|---|
| | | | | | Curve |
| | Instrument | Internal | | Monitored | Range |
| Analogue | (LC; MS) | Standard | Mobile Phase | Ions | (ng/mL) |
| OSU-ERβ- | Waters | Glyburide | A: 5:95:0.1 (v:v:v), | Negative | 0.1-5000 |
| 12 | UPLC BEH | | Acetonitrile:Water:Formic Acid | | |
| | C18 | | B: 50:50:0.1 (v:v:v), | | |
| | API-6500+ | | Acetonitrile:Methanol:Formic | | |
| | | | Acid | | |
| Compound | Waters | Glyburide | A: 100:0.1 (v:v), | Negative | 0.5-5000 |
| 8 | UPLC BEH | | Water:Formic Acid | | |
| | C18 | | B: 100:0.1 (v:v), | | |
| | API-6500 | | Acetonitrile:Formic Acid | | |
| Compound | Waters | Glyburide | A: 100:0.1 (v:v), | Negative | 0.5-5000 |
| 3 | UPLC BEH | | Water:Formic Acid | | |
| | C18 | | B: 100:0.1 (v:v), | | |
| | API-6500 | | Acetonitrile:Formic Acid | | |
| Compound | Ace Excel | Chrysin | A: 100:0.1 (v:v), | Negative | 0.5-5000 |
| 9 | C18-AR | | Water:Formic Acid | | |
| | API-5500 | | B: 100:0.1 (v:v), | | |
| | | | Acetonitrile:Formic Acid | | |
| Compound | Acquity | Chrysin | A: 100:0.1 (v:v), | Negative | 1-10000 |
| 7 | BEH C18 | | Water:Formic Acid | | |
| | API-5500 | | B: 100:0.1 (v:v), | | |
| | | | Acetonitrile:Formic Acid | | |
| Compound | Ace C18-AR | Chrysin | A: 100:0.1 (v:v), | Negative | 0.5-5000 |
| 11 | API-5500 | | Water:Formic Acid | | |
| | | | B: 100:0.1 (v:v), | | |
| | | | Acetonitrile:Formic Acid | | |
| Compound | Acquity | Glyburide | A: 100:0.1 (v:v), | Negative | 0.5-5000 |
| 10 | BEH C18 | | Water:Formic Acid | | |
| | API-6500+ | | | | |
| Compound | ACE Excel | Glyburide | B: 100:0.1 (v:v), | Negative | 0.5-5000 |
| 2 | C18-AR | | Acetonitrile:Formic Acid | | |
| | API-5500 | | | | |
| Compound | Waters | Glyburide | A: 100:0.1 (v:v), | Negative | 0.1-5000 |
| 5 | XBridge C8 | | Water:Formic Acid | | |
| | API-5500 | | | | |
| Compound | Waters | Glyburide | B: 100:0.1 (v:v), | Negative | 0.1-5000 |
| 6 | XBridge C8 | | Acetonitrile:Formic Acid | | |
| | API-5500 | | | | |
| Compound | Waters | Glyburide | A: 100:0.1 (v:v), | Negative | 0.1-5000 |
| 1 | XBridge C8 | | Water:Formic Acid | | |
| | API-5500 | | | | |
| Compound | Waters | Chrysin | B: 100:0.1 (v:v), | Negative | 0.1-5000 |
| 1 | XBridge C8 | | Acetonitrile:Formic Acid | | |
| | API-5500 | | | | |

Uterotrophic Assays. Analogs were assessed for uterotrophic activity in immature, estrogen-naïve female C57BL/6NJ mice (Jackson Laboratory, Bar Harbor, ME, USA). Mice were housed under conditions of constant photoperiod (12-h light/12-h dark), temperature, and humidity with ad libitum access to water and food. After weaning at 19 days of age, mice were fed a defined, phytoestrogen-free pelleted chow (AIN-93G, Dyets, Inc., Bethlehem, PA, USA). Mice were randomized to treatment groups (n=5 per group) that received test agents at doses ranging from 3 to 300 mg/kg, or vehicle (5% DMSO/5% Tween-20 in water [v/v] for OSU-ERβ-12; 20 or 4000 hydroxypropyl-β-cyclodextrin in water [w/w] for all others). Compounds were administered once daily by oral gavage for 4 days starting at 21 days of age. Positive control groups were administered estradiol (Steraloids, Newport, RI, USA) once daily by s.c. injection at 50 μg/kg (vehicle, 5% DMSO in corn oil, v/v). Weights were measured daily and mice were euthanized on the day after the last dose of the test agent. At necropsy, body weights were measured, and uteri were carefully excised, blotted to remove luminal fluid, and weighed. This study was conducted according to protocols approved by The Ohio State University Institutional Animal Care and Use Committee. Animal numbers were based on established guidance for uterotrophic assays (OECD, 2007) and internal, historical data.

Data Analyses and Statistical Methodologies. Data are presented as mean±SD/SEM (as indicated in the legends). Additionally, one-way ANOVA was used to assess trends in the data, and a p-value<0.05 was considered statistically significant. Statistical tests, plots, and regression fits were performed in GraphPad Prism version 9.4.1 for Windows, GraphPad Software, www.graphpad.com.

Transcriptional Activation Data: Non-normalized RLU values (FF-LUC) across at least n=3 independent experiments were fit to a three-parameter equation Y=Bottom+X*(Top−Bottom)/(EC₅₀+X) to determine EC₅₀ values and associated confidence intervals.

Uterotrophic Data: To more effectively compare across experiments, uterine tissue weights were first normalized to body weight on a per animal basis. The mean value for untreated control tissue weights were then subtracted from each BW-normalized tissue weight and the subtracted values represented as a percentage of the mean value of the estradiol positive control group, on a per experiment basis. These data were then fit to the equation Y=100*X/(EC₅₀+X) to determine uterotrophic EC₅₀ dose levels and associated confidence intervals.

### Results

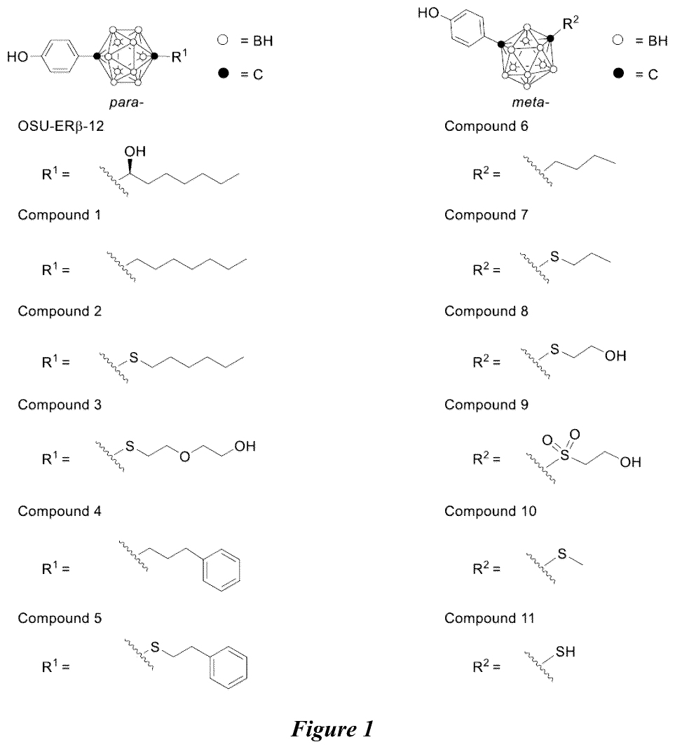

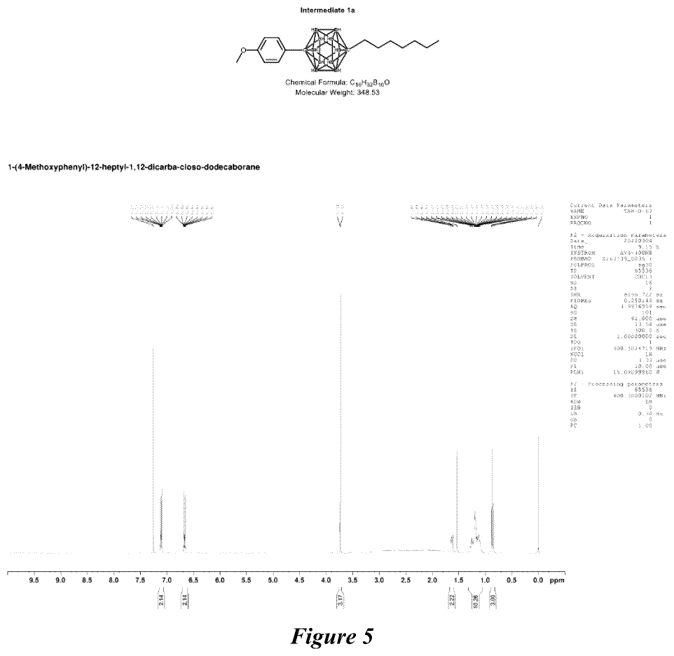

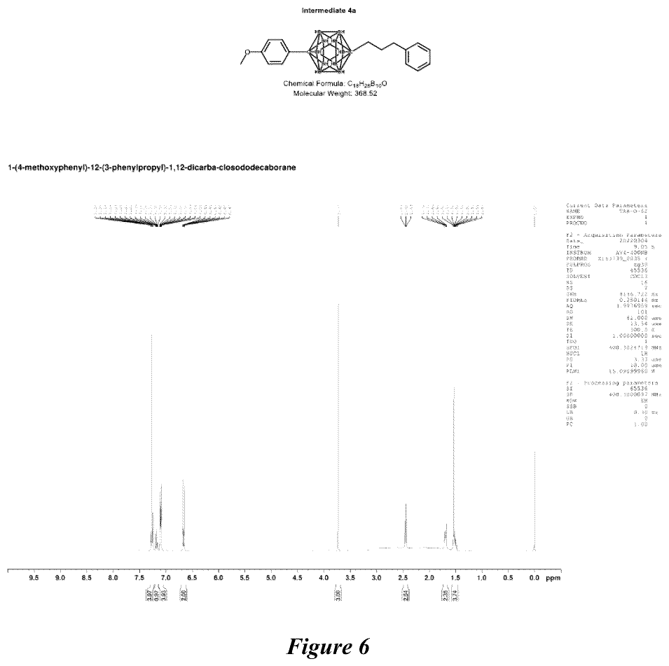

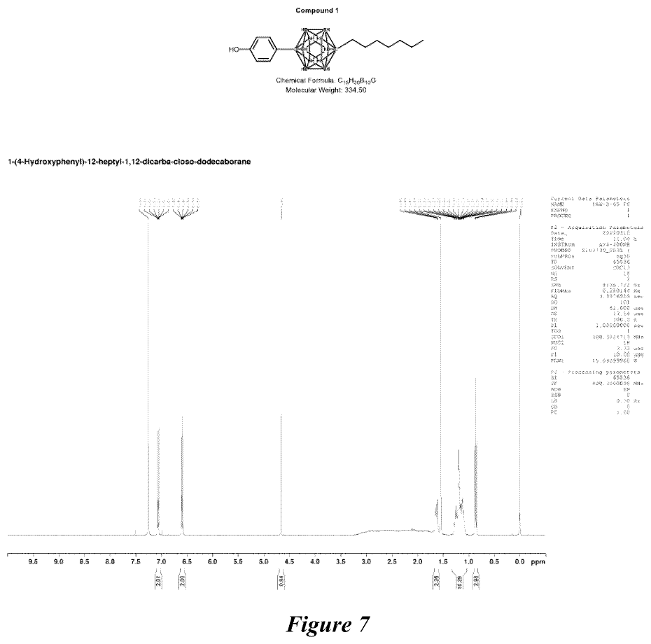

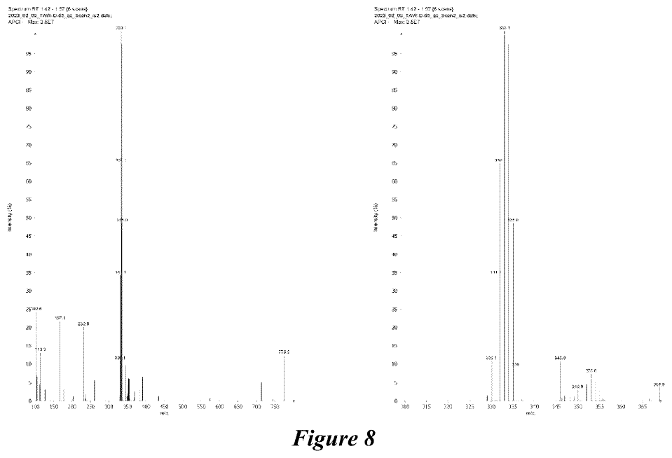

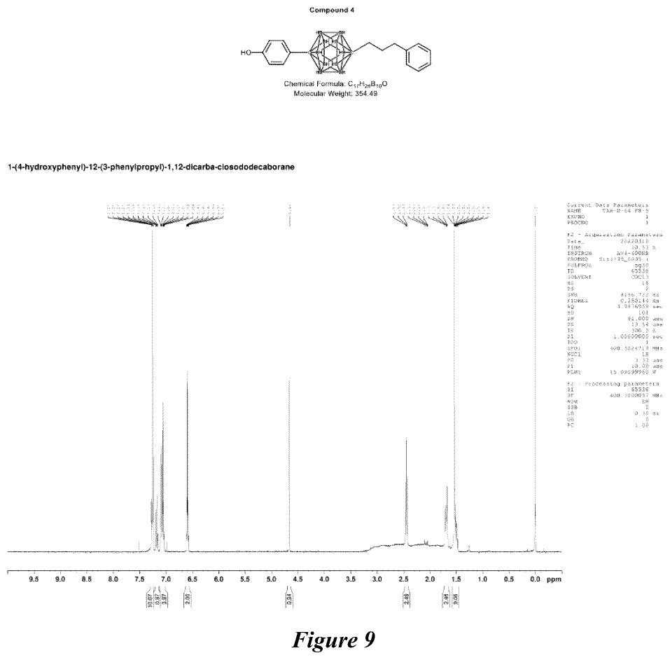

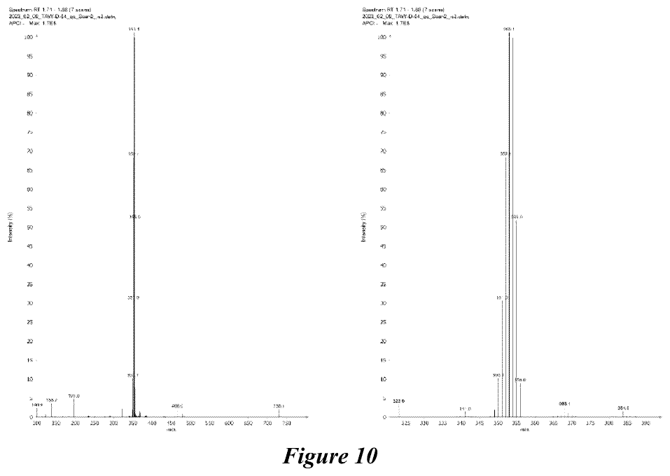

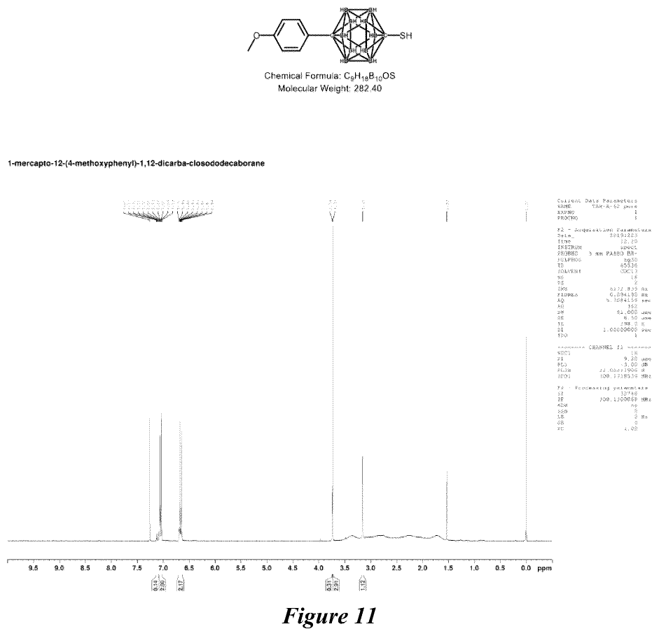

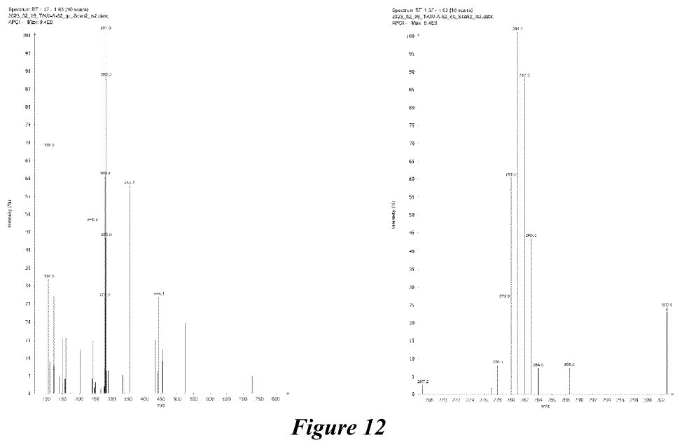

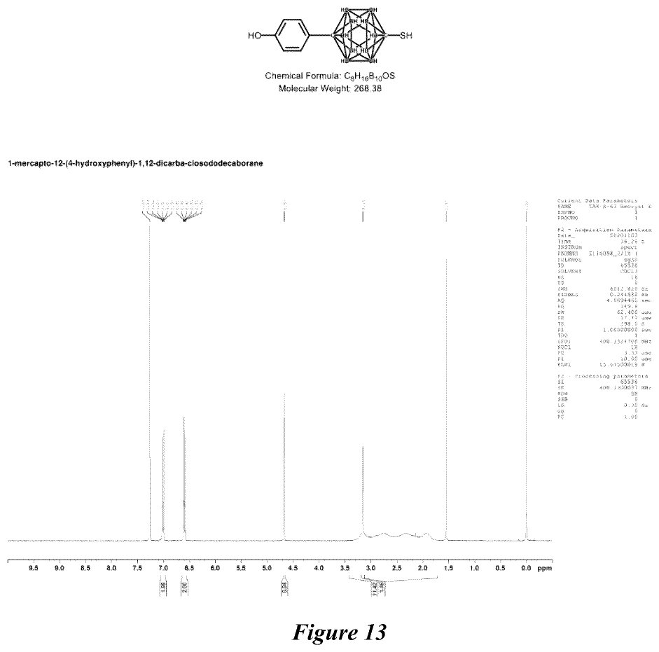

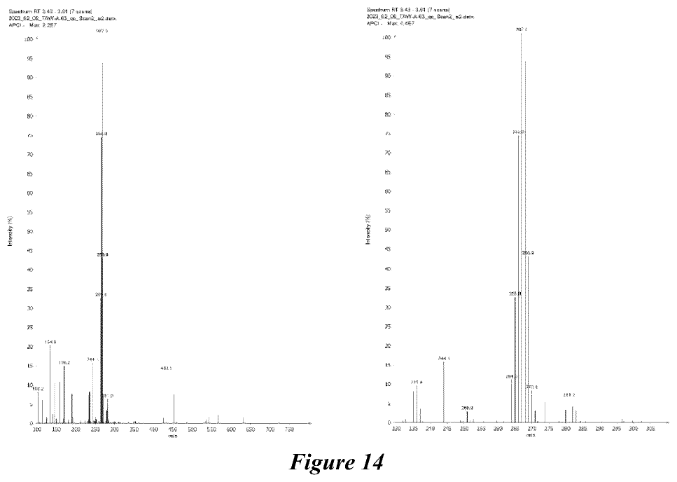

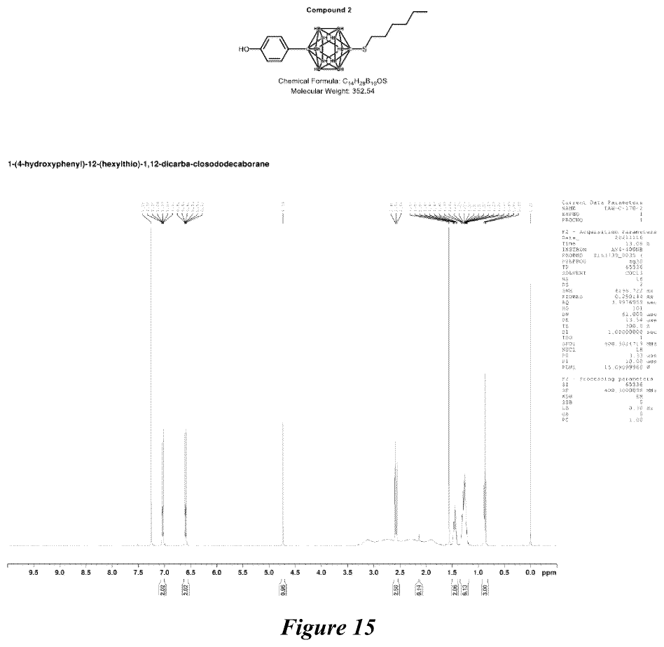

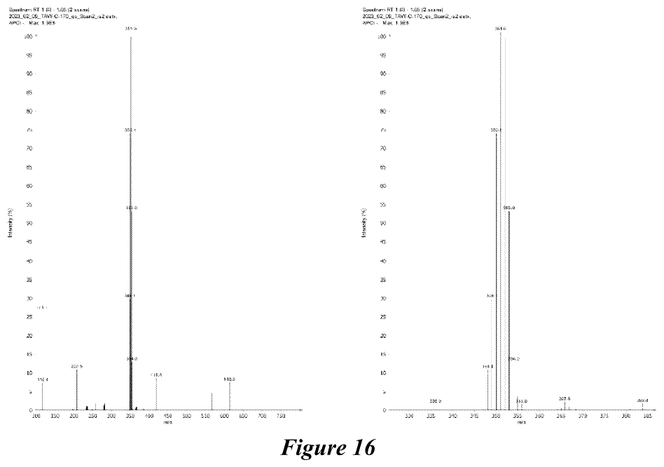

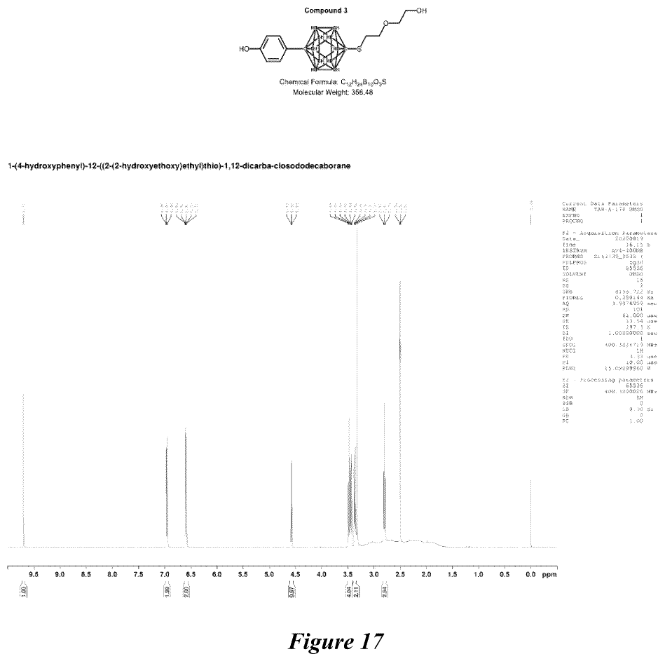

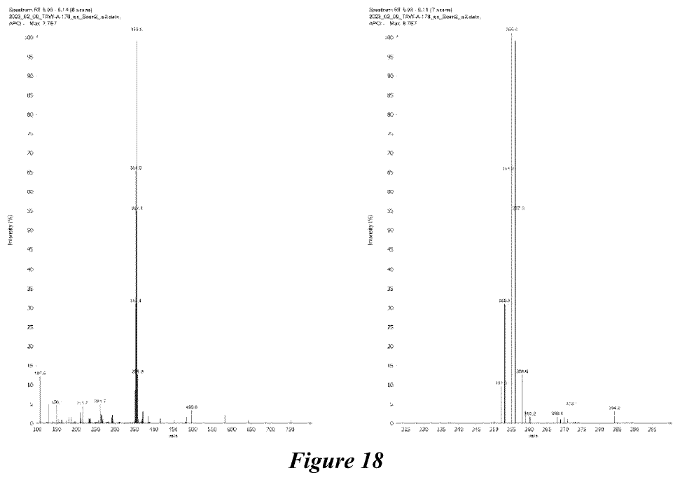

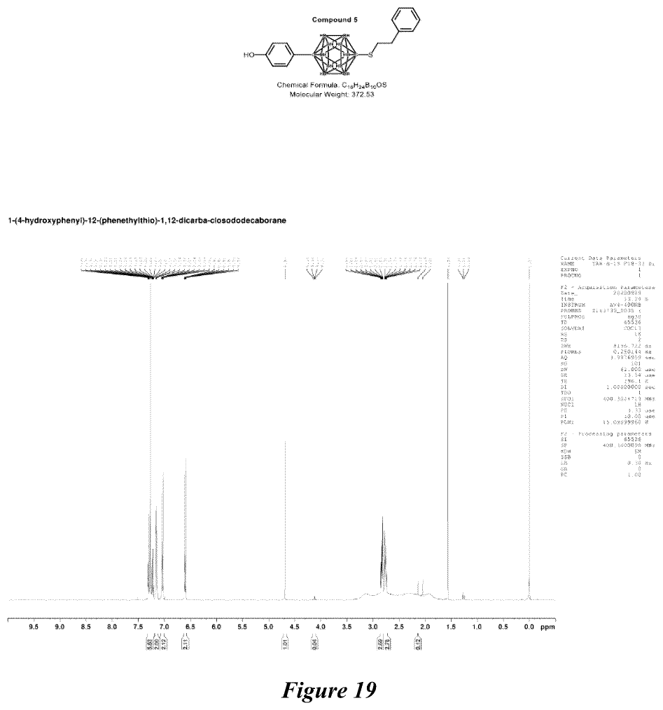

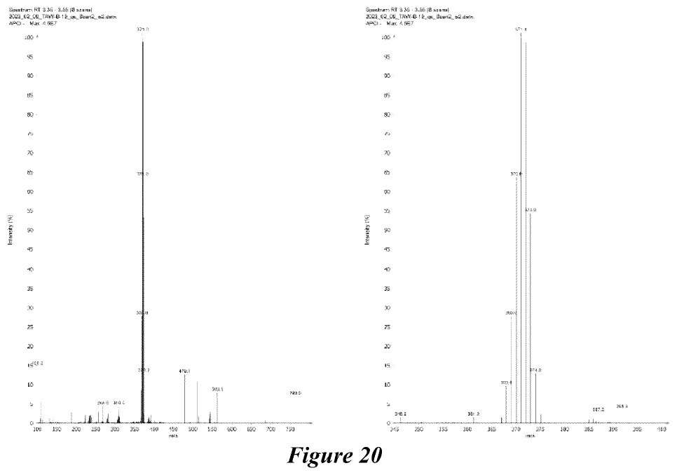

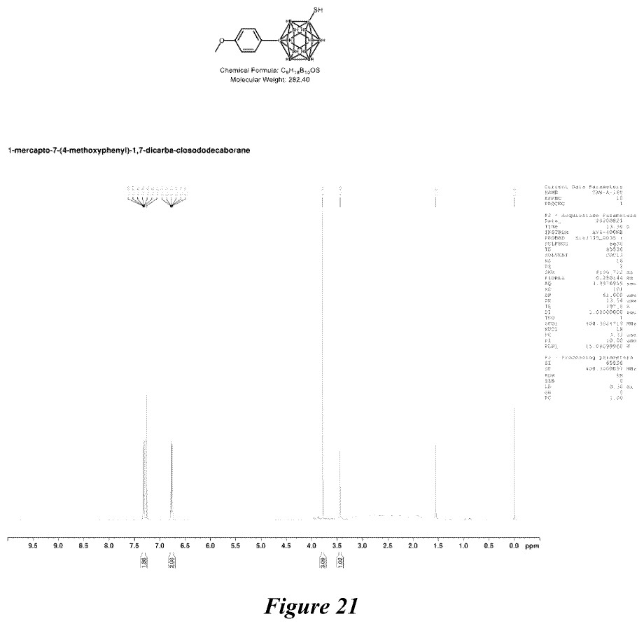

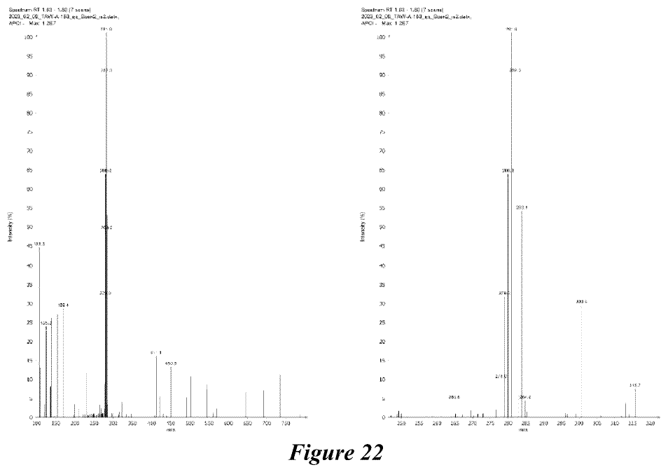

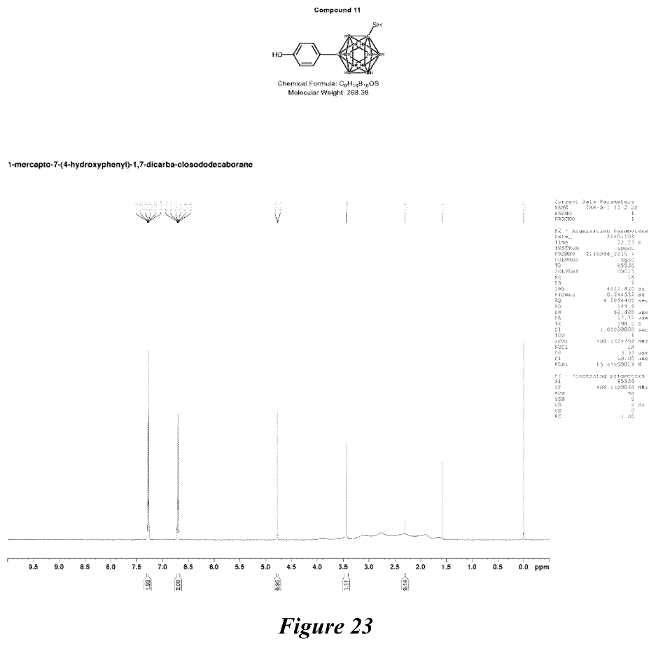

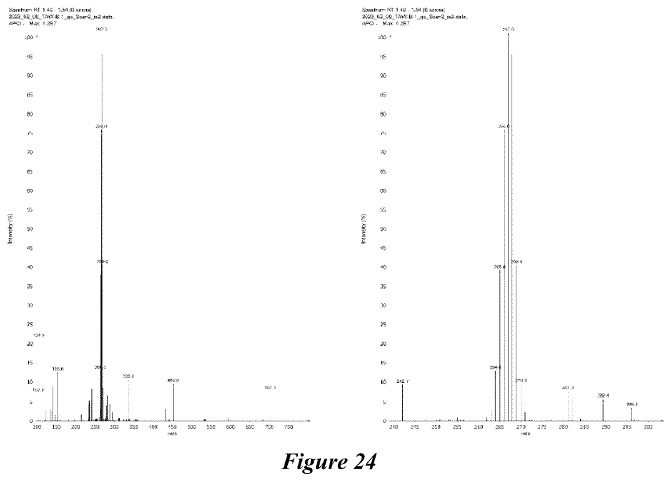

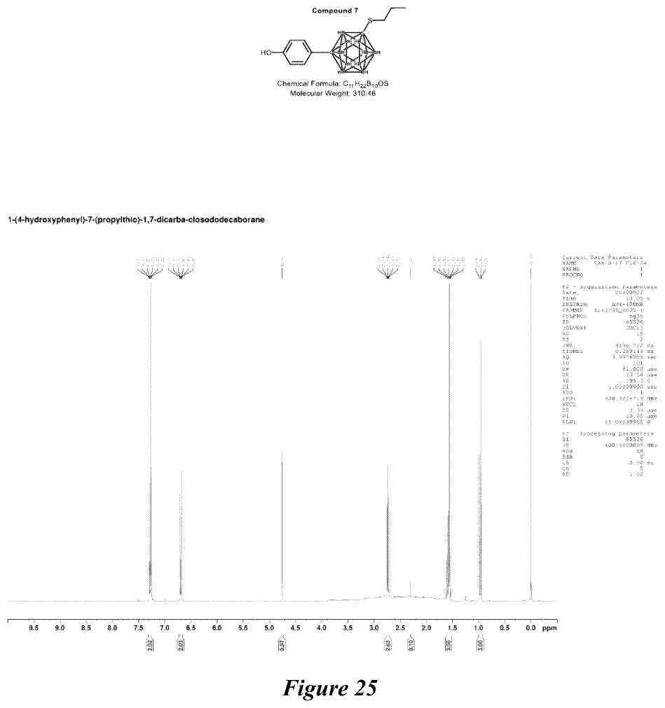

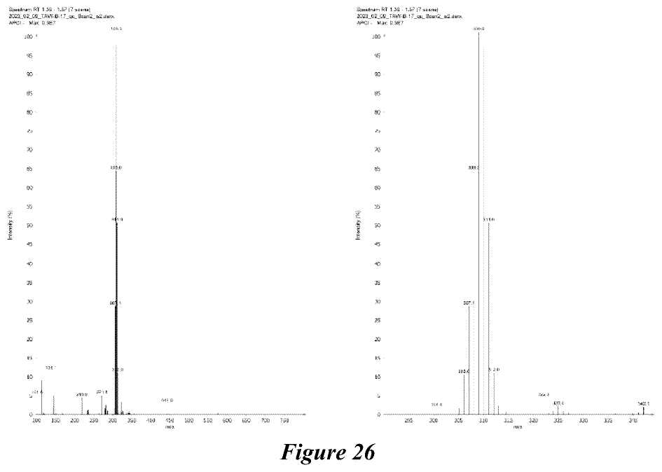

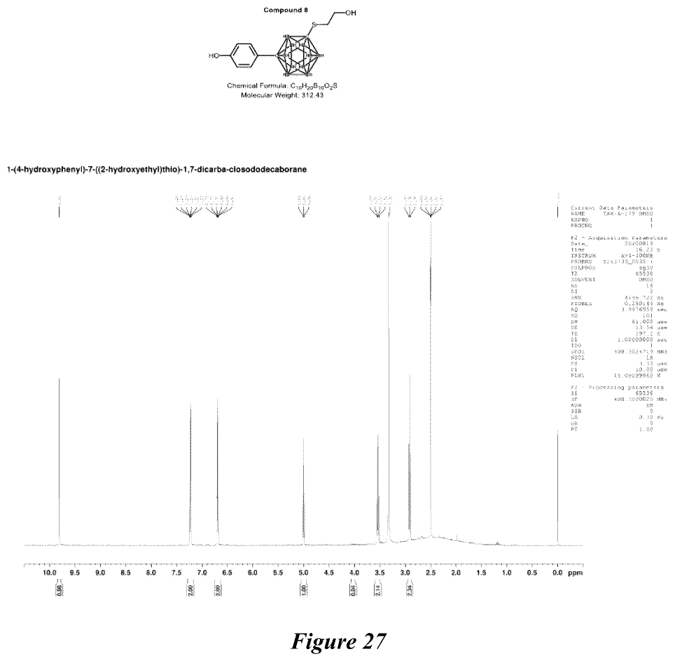

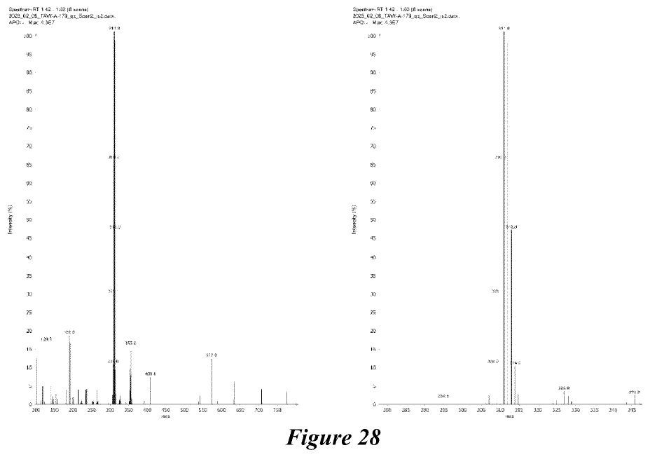

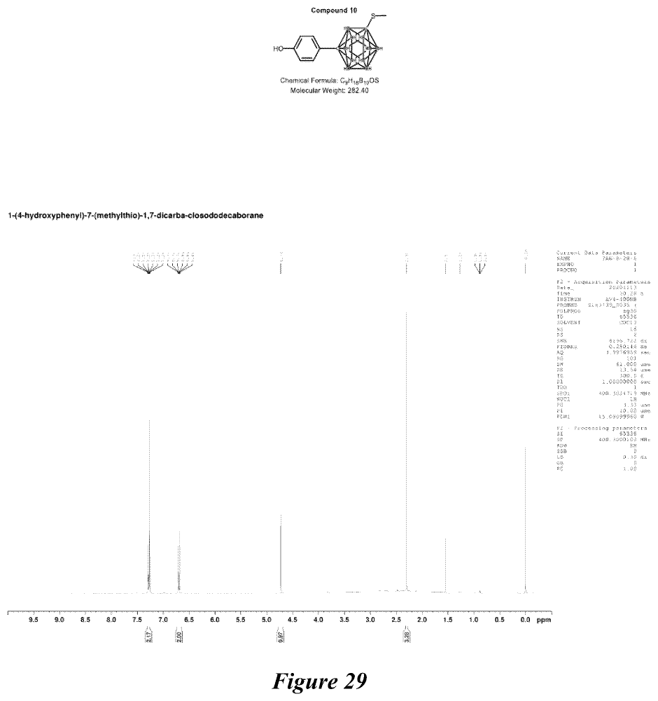

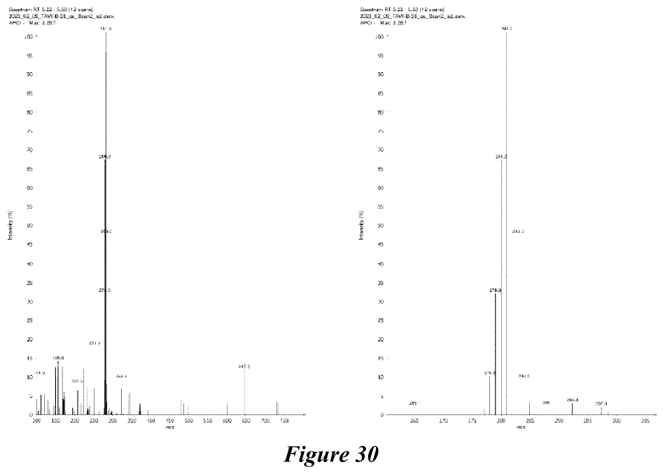

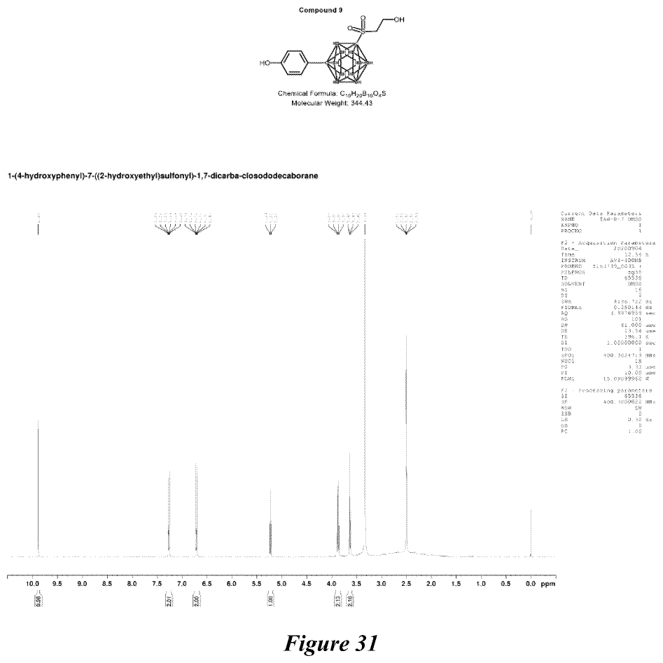

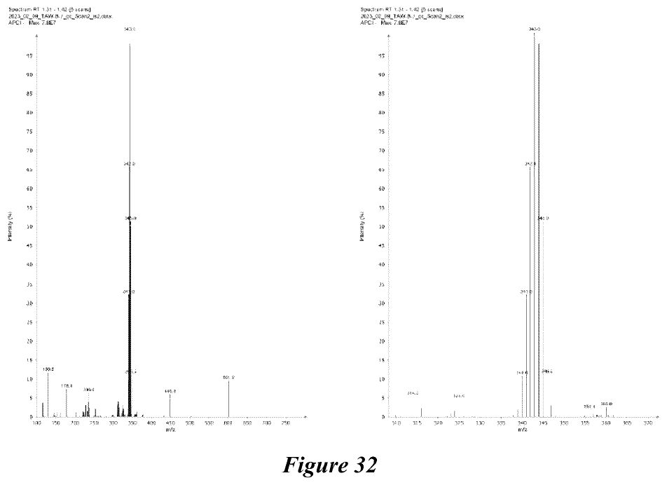

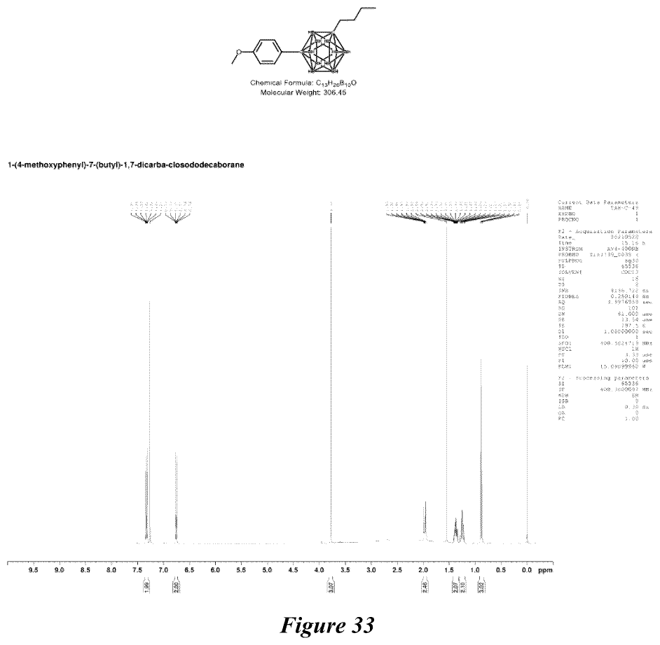

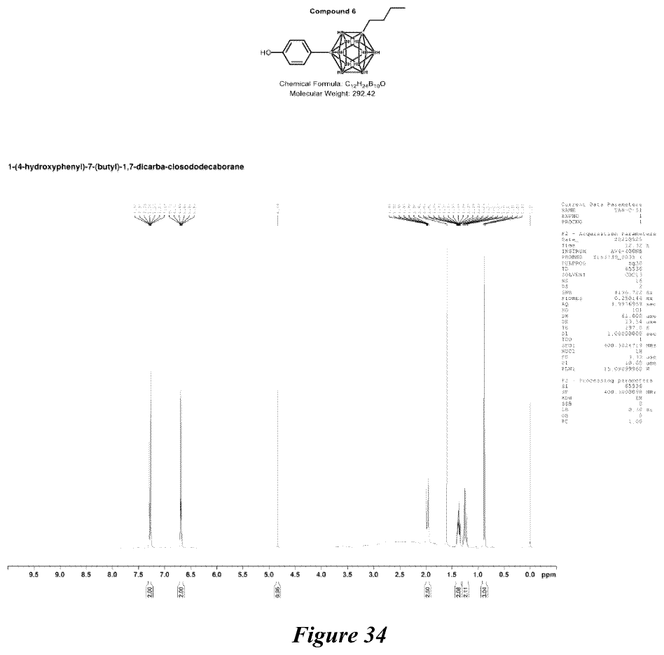

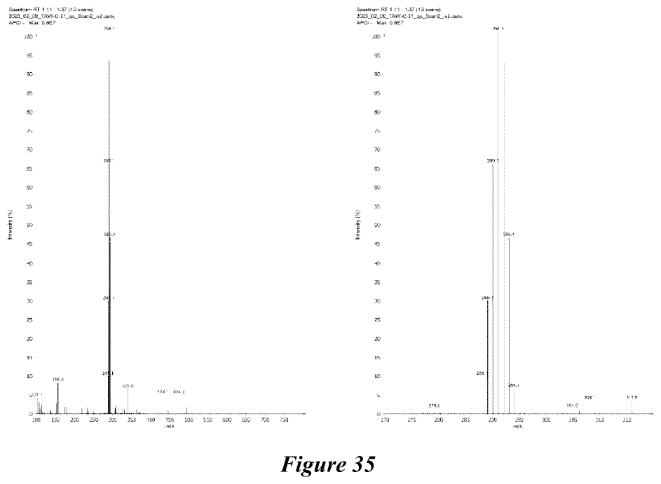

Chemistry. The chemical structure of analogs in the para- and meta-series are shown in FIG. 1. The synthetic protocol for each analog is provided above and results are shown in FIG. 5-FIG. 35.

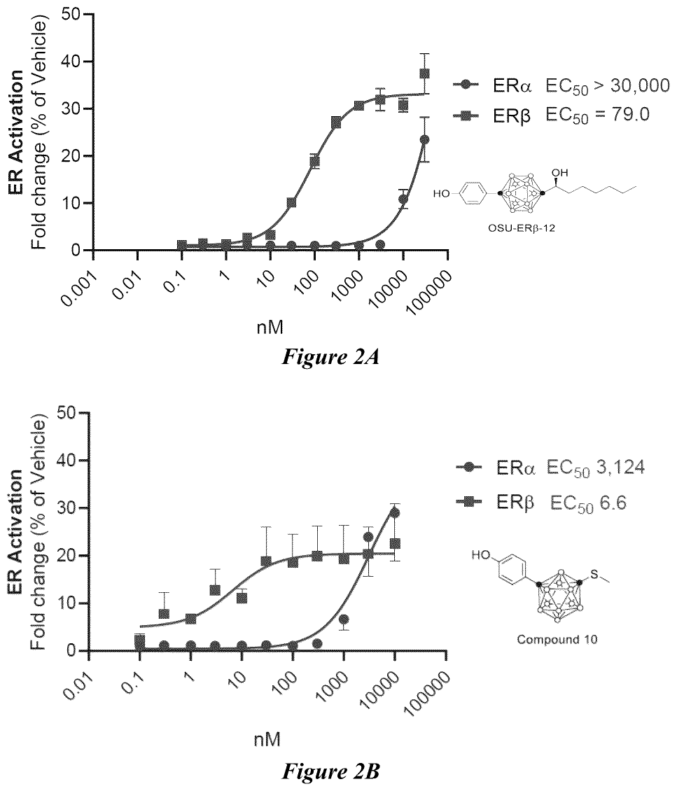

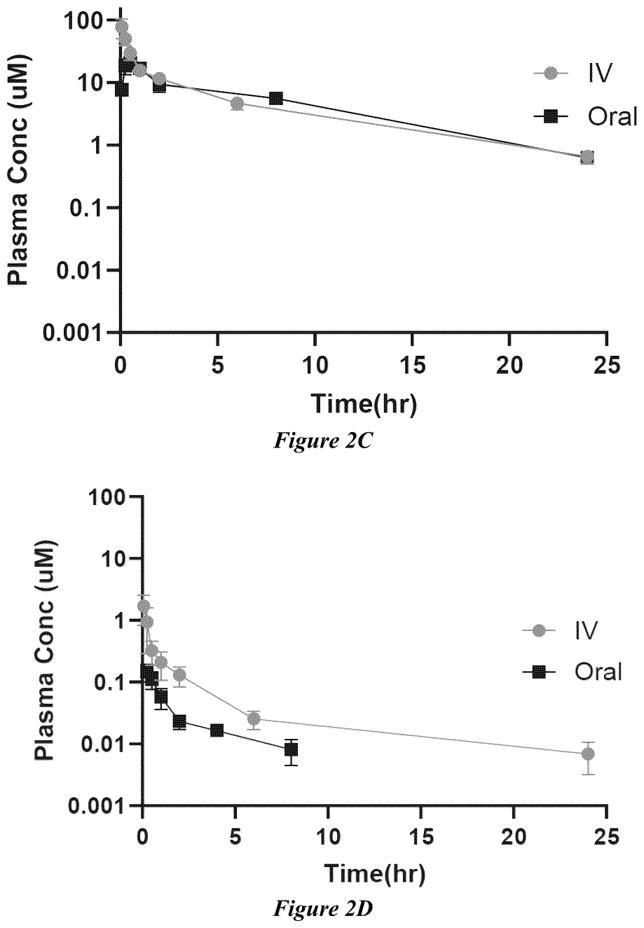

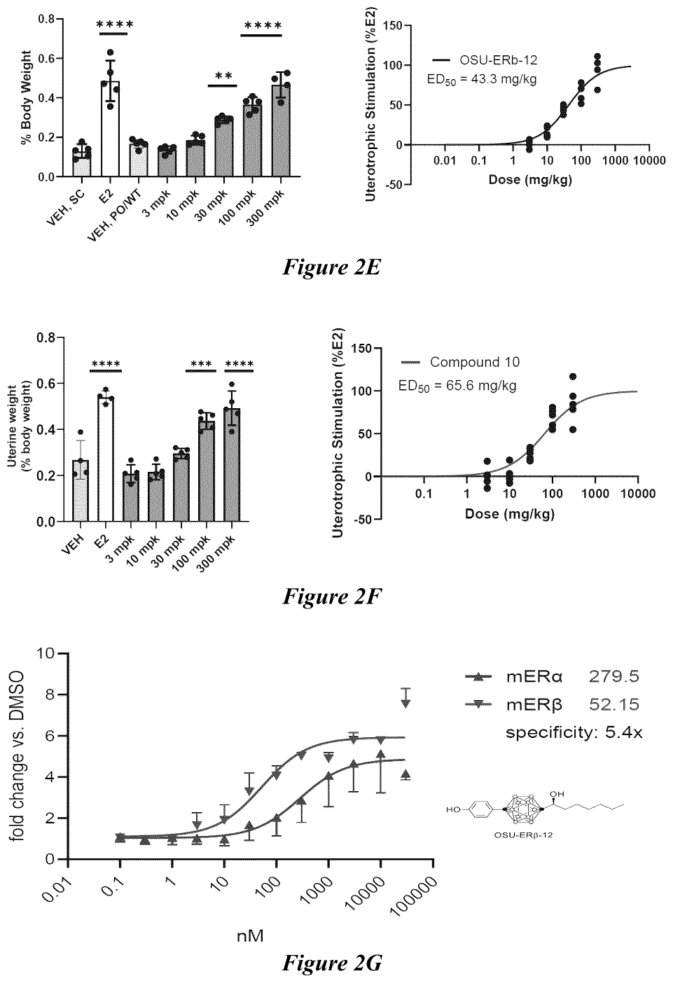

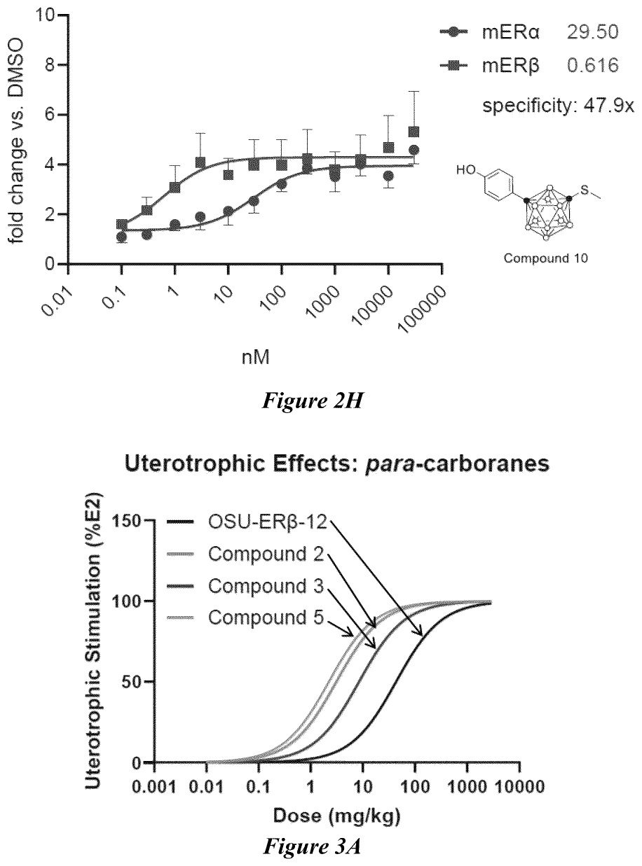

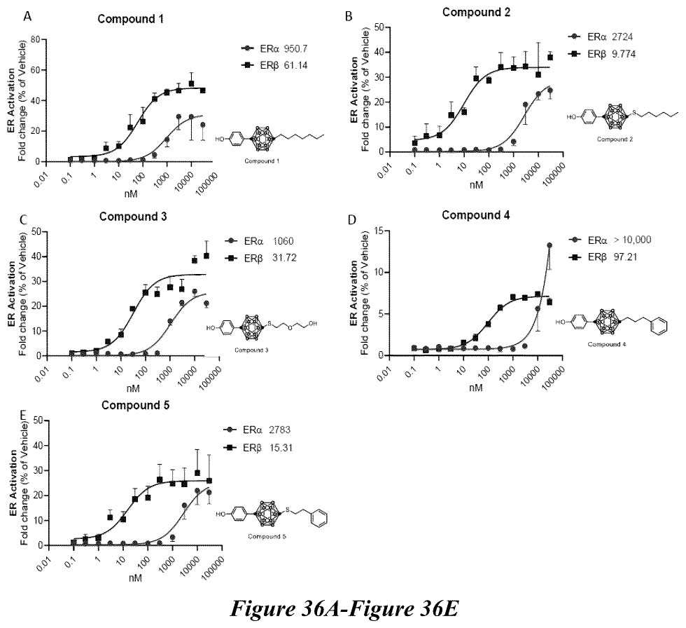

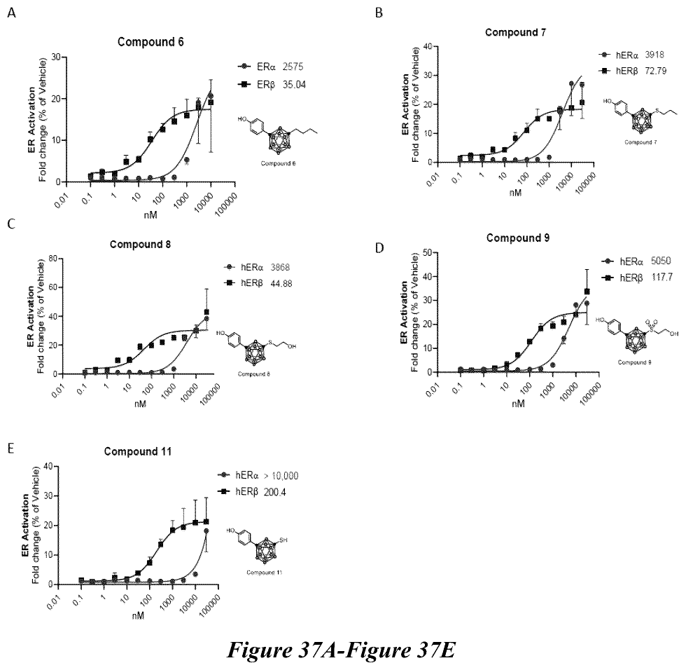

In Vitro Structure-Activity Relationship. To evaluate the cell-free human ER binding and in vitro functional properties of the para- and meta-substituted analogs, competitive radioligand binding and transcriptional activation assays were employed, respectively (Table 4A-Table 4B, FIG. 2A-FIG. 2B, FIG. 36A-FIG. 36E, and FIG. 37A-FIG. 37E).

TABLE 4A
Structures and human ERa/β ligand binding and transactivation activities of para-
substituted analogs.

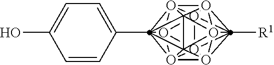

| | | | | | Transactivation^{b} | | |
|---|---|---|---|---|---|---|---|
| | | | | | | hERB | |
| | | Binding Affinity^{a} | | | hERa | EC₅₀, | |
| | | hERα | hERβ | | EC₅₀, | nM | |
| | | Ki | Ki | Binding | nM | (95% | |
| Compound | R¹ | (nM) | (nM) | Selectivity | (95% CI) | CI) | Selectivity |
| OSU-ERβ- 12 | 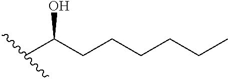 [SMILES: *[C@@H](O)CCCCCC] | 6.6 | 2.0 | 3.3 | >30,000 | 79.0 (60.1- 104) | >376 |
| Compound 1 | 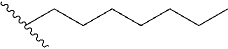 [SMILES: *CCCCCCC] | 43.0 | 20.0 | 2.2 | 950.7 (460.6- 1,911) | 61.1 (35.6- 104.1) | 15.5 |
| Compound 2 | 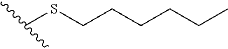 [SMILES: *SCCCCCC] | 9.6 | 4.6 | 2.1 | 2,724 (1,694- 4,427) | 9.7 (4.3- 21.8) | 281 |
| Compound 3 | 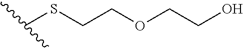 [SMILES: *SCCOCCO] | 2.4 | 0.8 | 3.2 | 1,060 (678- 1,653) | 31.7 (14.0- 77.9) | 33 |
| Compound 4 | 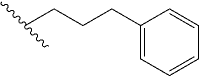 [SMILES: *CCCc1ccccc1] | 22.0 | 12.7 | 1.7 | >10,000 | 97.2 (72.5- 130.0) | >102.8 |
| Compound 5 | 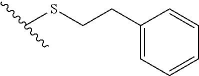 [SMILES: *SCCc1ccccc1] | 14.6 | 3.5 | 4.2 | 2,783 (1,684- 4,649) | 15.3 (5.1- 45.0) | 182 |

^{a}Compound affinities for human ERα or ERβ proteins were evaluated in radioligand-based competitive binding assays. Binding selectivity was calculated as a ratio of the Ki for hERα/hERβ.
^{b}Each compound was tested for human ERα and ERβ agonism in a luciferase transactivation assay. The EC₅₀ values are shown in nanomolar. Transactivation assay selectivity was calculated as a ratio of the EC₅₀ for hERα/hERβ.

TABLE 4B
Structures and human ERα/β ligand binding and transactivation activities of meta-
substituted analogs.

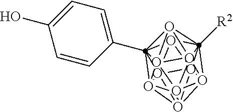

| | | Binding Affinity^{a} | | | Transactivation^{b} | | |
|---|---|---|---|---|---|---|---|
| | | hERα | hERβ | | hERα | hERβ | |
| | | Ki | Ki | Binding | EC₅₀, nM | EC₅₀, nM | |
| Compound | Structure/R² | (nM) | (nM) | Selectivity | (95% CI) | (95% CI) | Selectivity |
| OSU-Erβ- 12 | 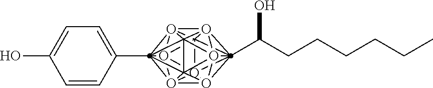 | 6.6 | 2.0 | 3.3 | >30,000 | 79.8 (60.1- 104) | >376 |

Meta series

| | | | | | | | |
|---|---|---|---|---|---|---|---|
| Compound 6 | 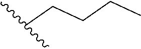 [SMILES: *CCCC] | 16.5 | 4 | 4.1 | 2,575 (1,067- 7,161) | 35.0 (17.8- 70.7) | 74 |
| Compound 7 | 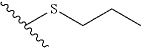 [SMILES: *SCCC] | 26.0 | 5.0 | 5.2 | 3,918 (2,044- 7,733) | 72.8 (29.9- 178.7) | 53.8 |
| Compound 8 | 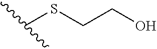 [SMILES: *SCCO] | 4.3 | 0.5 | 8.6 | 3,868 (2,567- 5,594) | 44.9 (10.95- 283.9) | 86.2 |
| Compound 9 | 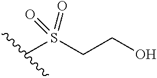 [SMILES: *S(=O)(=O)CCO] | 11.8 | 1.6 | 7.4 | 5,050 (3,332- 7,753) | 118 (61.3- 231.9) | 43 |
| Compound 10 | 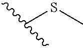 [SMILES: *SC] | 50.0 | 5.9 | 8.4 | 3,124 (1,639- 6,459) | 6.6 (17.7- 23.5) | 473 |
| Compound 11 | 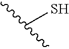 [SMILES: *S] | 200 | 55 | 3.6 | >10,000 | 200 (99.2- 403) | >50 |

^{a}Compound affinities for human ERα or ERβ proteins were evaluated in radioligand-based competitive binding assays. Binding selectivity was calculated as a ratio of the Ki for hERα/hERβ.
^{b}Each compound was tested for human ERa and ERβ agonism in a luciferase transactivation assay. The EC₅₀ values are shown in nanomolar. Transactivation assay selectivity was calculated as a ratio of the EC₅₀ for hERα/hERβ.

### Para-Carborane Series

Consistent with a previous report using slightly different assay systems (Sedlák et al., 2021), the chiral alcohol in the first-position of the alkyl chain of OSU-ERβ-12 was required for potent binding to hERβ and both selective binding and activation over hERα (OSU-ERβ-12 versus Compound 1, Table 4A, FIG. 2A, FIG. 36A-FIG. 36E). Isosteric replacement of the first carbon in the alkyl chain of Compound 1 with sulfur, restored potent hERβ binding and activation over hERα for Compound 2, relative to Compound 1, demonstrating similar selectivity to OSU-ERβ-12 but greater hERβ potency in transactivation (EC₅₀'s of 9.7 versus 61.1 nM, respectively). The insertion of an additional oxygen and terminal hydroxyl group to Compound 2 (see Compound 3), while keeping the chain length the same, resulted in increased potency in both hERβ and hERα binding affinity but reduced hERβ selectivity in transactivation. Also consistent with the previous report, terminating the alkyl chain with an unsubstituted aromatic ring (Compound 4) was associated with reductions in hERβ binding potency and selectivity, which was similar to the unsubstituted alkyl chain compound (Compound 1). However, the terminal phenyl maintained hERβ selectivity in transcriptional activation. Lastly, the sulfide Compound 5, an isostere of Compound 4, exhibited improved hERβ potency and selectivity in both binding and transcriptional activation.

### Meta-Carborane Series

A similar investigation was carried out using various substituted meta-substituted carboranes. When the unsubstituted alkyl chain was shortened to 4 carbons and oriented in the meta position of the carborane, both potent and selective hERβ binding and transactivation were apparent (Compound 6, Table 4B, FIG. 37A-FIG. 37E), in stark contrast to Compound 1 (Table 4A). Compound 7, the sulfur containing isostere of Compound 6 afforded no obvious improvements in desired hERβ activity. Terminating the alkyl chain with an —OH (Compound 8) resulted in the most potent and selective hERβ binder in the series (0.5 nM or a 4-fold improvement over OSU-ERβ-12 binding and 8.6 or a 2.5-fold improvement over OSU-ERβ-12 isotype selectivity, respectively). Compound 8 also retained potency and selectivity in hERβ transactivation. Oxidation of the sulfide of Compound 8 to a sulfone provided Compound 9 which resulted in small reductions in hERβ binding potency and selectivity but a larger reduction in both hERβ potency and selectivity in transactivation. Reducing the chain length of Compound 7 by 2 carbons (Compound 10, Table 4B, FIG. 2B) demonstrated similar hERβ binding potency but greater selectivity and the largest improvements in both hERβ potency and selectivity in transactivation in the series. However, removing the alkyl chain completely to unmask the free thiol (Compound 11) resulted in 10-fold reductions in hERβ binding and matching reductions in potency and selectivity in transactivation.

### Mouse Pharmacokinetic Studies

### Para-Carborane Series

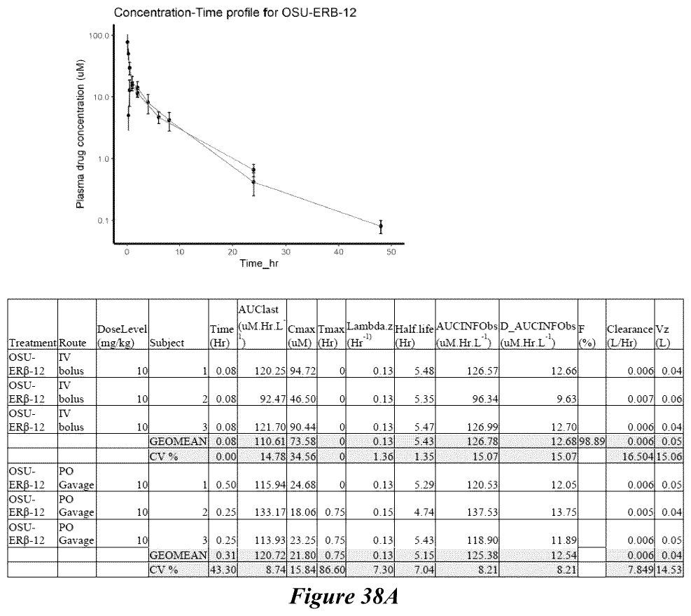

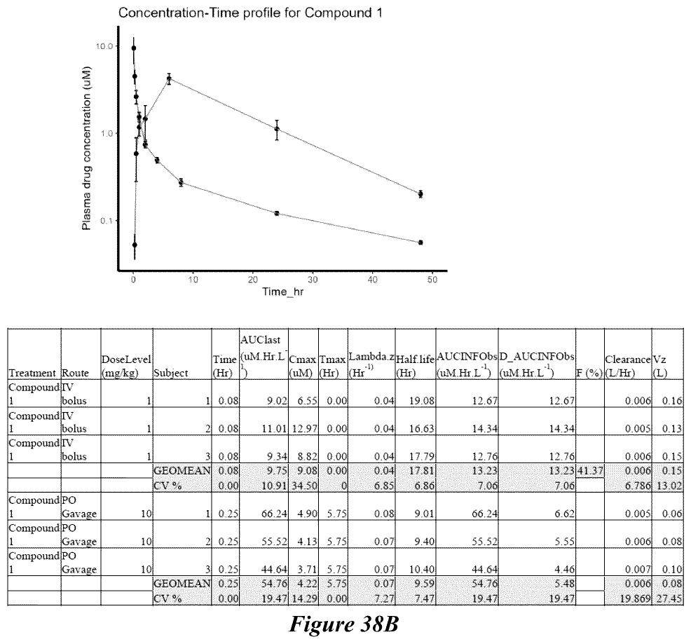

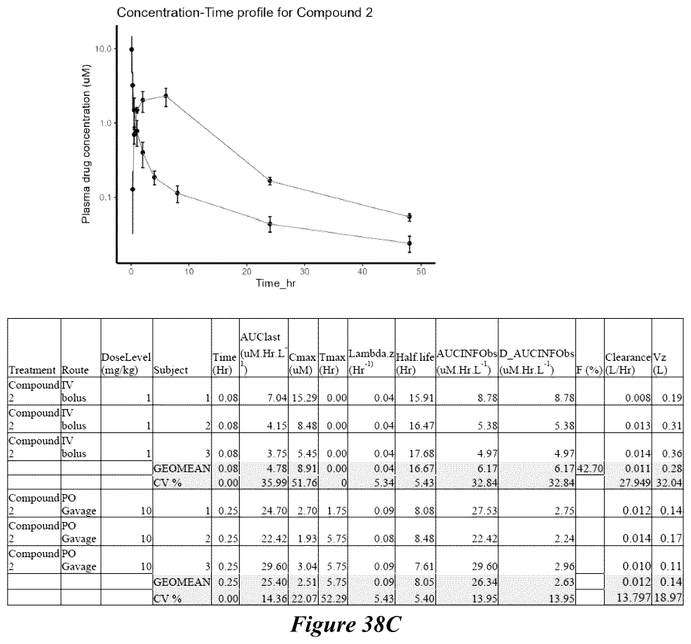

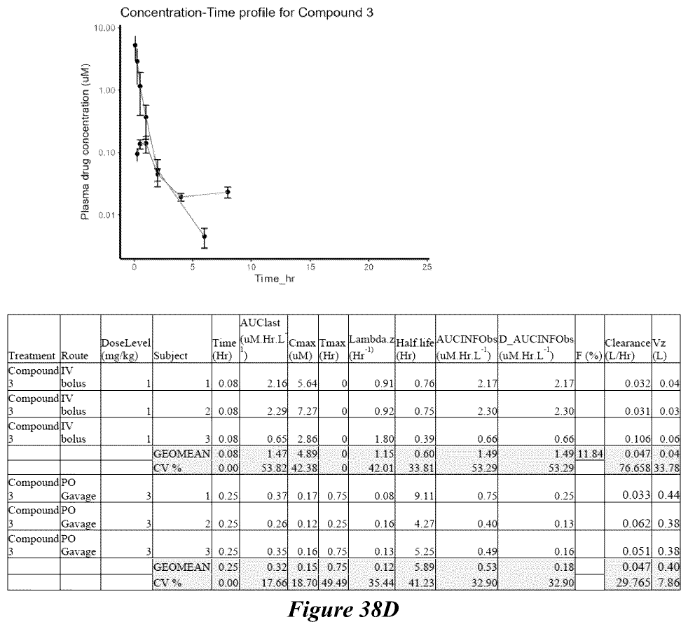

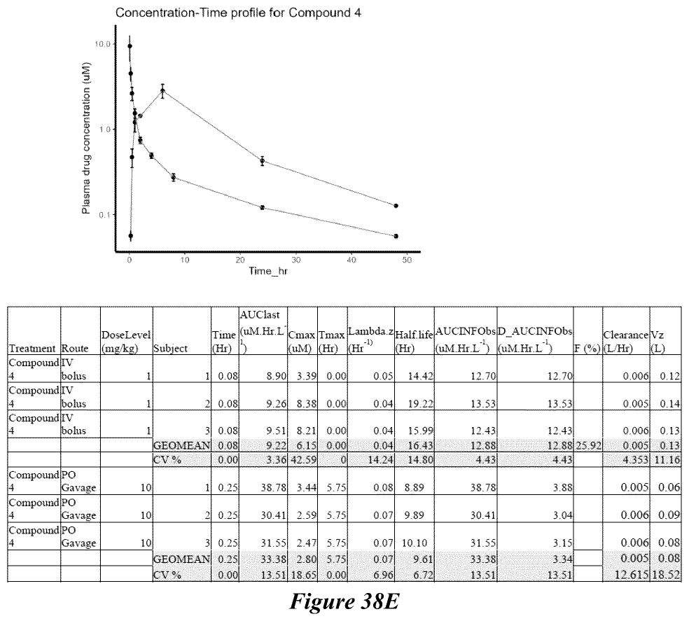

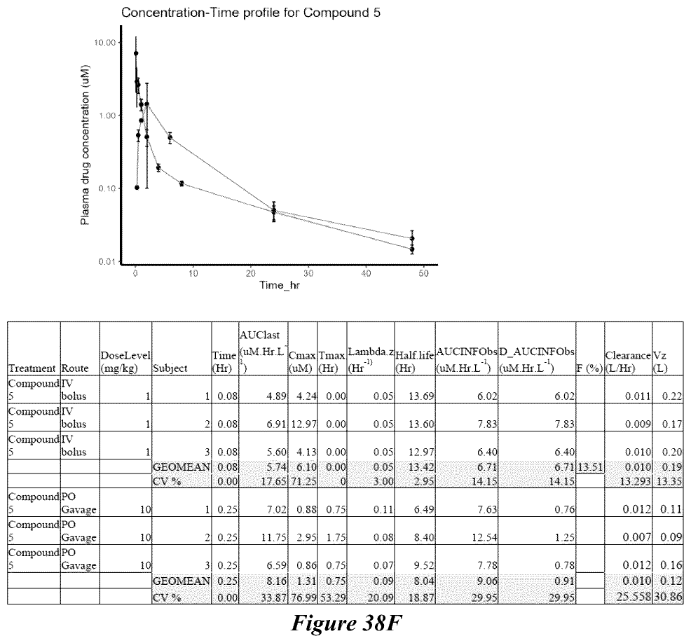

To assess the in vivo stability and potential suitability for oral dosing single dose IV and PO pharmacokinetic studies of the ligands were performed in C57BL/6 mice (Table 5A-Table 5B, FIG. 38A-FIG. 38F). Following an IV dose, OSU-ERβ-12 demonstrated very low systemic clearance (CL) in mice of 0.006 L/h, or less than 5% of total hepatic blood flow (Davies & Morris, 1993), and an elimination half-life of 5.4 hours (Table 5A, FIG. 2C, FIG. 38A). The analog with an unsubstituted alkyl chain (Compound 1) had a similarly low systemic CL following an IV dose, but a substantially longer elimination half-life of nearly 18 hours (Table 5A, FIG. 38B). The sulfur-containing isostere analog of Compound 1 (Compound 2, FIG. 38C), had nearly double the systemic clearance of Compound 1 but a similar elimination half-life of 16.7 hours. Replacement of the central and terminal carbons with oxygen in the alkyl chain of Compound 2 to make Compound 3 (FIG. 38D) increased IV clearance an additional 4-fold (0.047 L/hr), or more than 8-fold compared to OSU-ERβ-12, and drastically reduced elimination half-life to 0.6 hours. The terminal phenyl ring analog, Compound 4 (FIG. 38E), had very similar IV pharmacokinetics as compared to the unsubstituted alkyl chain Compound 1. Whereas the sulfide analog of Compound 4, Compound 5 (FIG. 38F) had nearly double the IV systemic CL. and a slightly shorter elimination half-life of 13.4 hours.

Following an oral dose, OSU-ERβ-12 was rapidly and completely absorbed (F %=98.9) with a Tₘₐₓ of 0.75 hours and a Cₘₐₓ of 21.8 μM, resulting in an oral dose adjusted AUC_{D_INF} of 12.5 μM*hr (Table 5B, FIG. 38A). Dose adjusted Cₘₐₓ values ranged from 5-fold lower for the unsubstituted alkyl chain analog Compound 1 (4.2 μM) to more than 40-fold lower for the ethylene glycol-containing analog Compound 3 (0.2 μM at 3 mg/kg). AUC_{D_INF} values followed this pattern with values ranging from 2.2-fold lower for Compound 1 (5.5 μM*hr) to almost 70-fold lower for Compound 3 (0.2 μM*hr at 3 mg/kg).

TABLE 5A
Mouse IV pharmacokinetic parameter estimates for para-substituted analogs.

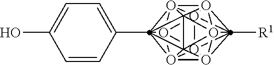

| | | | Clearance, | | Elimination T_{1/2}, |
|---|---|---|---|---|---|
| | | Dose | L/hr | V_{d}, L | hr |
| Compound | R¹ | (mg/kg) | (CV %) | (CV %) | (CV %) |
| OSU-Erβ-12 | 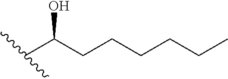 [SMILES: *[C@@H](O)CCCCCC] | 10 | 0.006 (16.5) | 0.05 (15.0) | 5.4 (1.35) |
| Compound 1 | 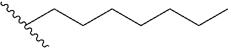 [SMILES: *CCCCCCC] | 1 | 0.006 (6.7) | 0.15 (13.0) | 17.8 (6.86) |
| Compound 2 | 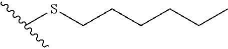 [SMILES: *SCCCCCC] | 1 | 0.011 (27.9) | 0.277 (32.0) | 16.7 (5.43) |
| Compound 3 | 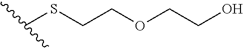 [SMILES: *SCCOCCO] | 1 | 0.047 (76.6) | 0.041 (33.8) | 0.6 (33.8) |
| Compound 4 | 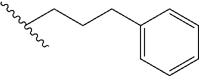 [SMILES: *CCCc1ccccc1] | 1 | 0.005 (4.4) | 0.13 (11.16) | 16.4 (14.8) |
| Compound 5 | 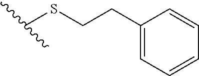 [SMILES: *SCCc1ccccc1] | 1 | 0.01 (13.3) | 0.194 (13.3) | 13.4 (2.95) |

The IV pharmacokinetic characteristics of each compound were determined after single-dose injections in mice followed by serial blood draws. The pharmacokinetic parameter estimates were obtained by non-compartmental analysis.
V_{d}, volume of distribution; T_{1/2}, half-life; CV, coefficient of variation

TABLE 5B
Mouse PO pharmacokinetic parameter estimates for para-substituted analogs.

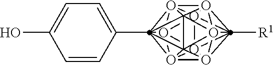

| | | | Cmax, | Tmax, | AUC_{D}_INF, | | Elimination |
|---|---|---|---|---|---|---|---|
| | | Dose | μM | hr | μM*hr | F | T_{1/2}, hr |
| Compound | R¹ | (mg/kg) | (CV %) | (CV %) | (CV %) | (%) | (CV %) |
| OSU-Erβ- 12 | 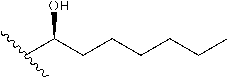 [SMILES: *[C@@H](O)CCCCCC] | 10 | 21.8 (15.8) | 0.75 (86.6) | 12.5 (8.2) | 98.9 | 5.4 (7.04) |
| Compound 1 | 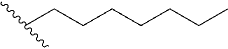 [SMILES: *CCCCCCC] | 10 | 4.2 (14.3) | 5.75 (0.00) | 5.5 (19.5) | 41.4 | 9.6 (7.5) |
| Compound 2 | 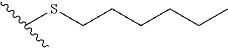 [SMILES: *SCCCCCC] | 10 | 2.5 (22.1) | 5.75 (52.3) | 2.6 (13.95) | 41.7 | 8.1 (5.4) |
| Compound 3 | 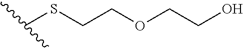 [SMILES: *SCCOCCO] | 3 | 0.2 (18.7) | 0.75 (49.49) | 0.2 (32.9) | 11.9 | 5.9 (41.2) |
| Compound 4 | 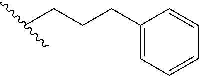 [SMILES: *CCCc1ccccc1] | 10 | 2.8 (18.7) | 5.75 (0.0) | 3.3 (13.5) | 25.9 | 9.6 (6.7) |
| Compound 5 | 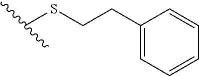 [SMILES: *SCCc1ccccc1] | 10 | 1.3 (76.9) | 0.75 (53.3) | 0.9 (30) | 13.5 | 8.0 (18.9) |

The oral pharmacokinetic characteristics of each compound were determined after single-dose oral administration in mice followed by serial blood draws. The pharmacokinetic parameter estimates were obtained by non-compartmental analysis.
Cmax, maximum concentration; Tmax, time of Cmax; AUC_{D}_INF, Dose-adjusted AUC infinity; F, Absolute bioavailability; T_{1/2}, half-life; CV, coefficient of variation

### Meta-Carborane Series

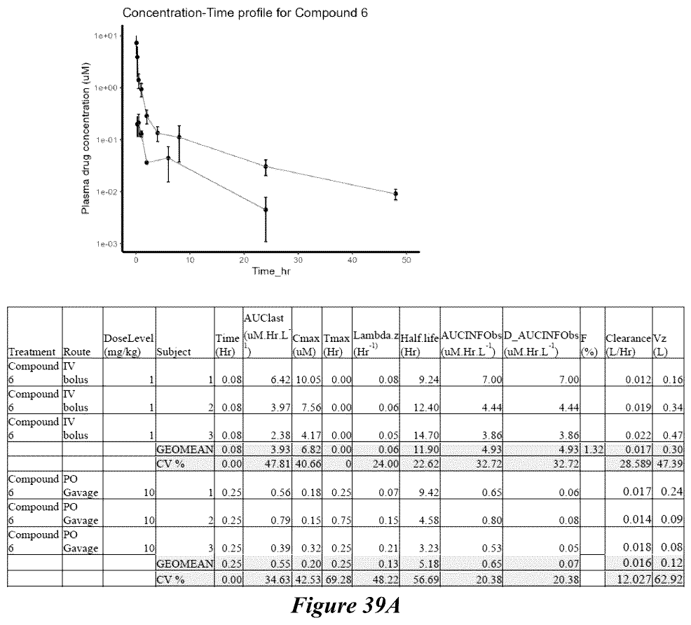

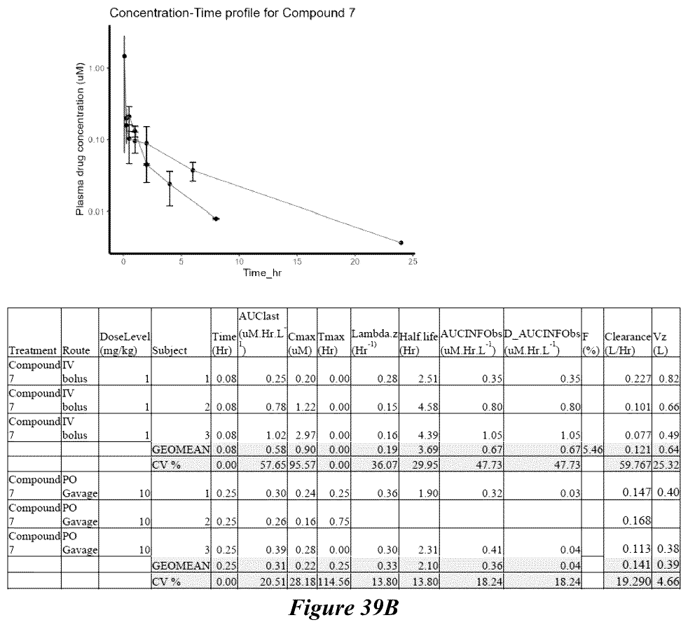

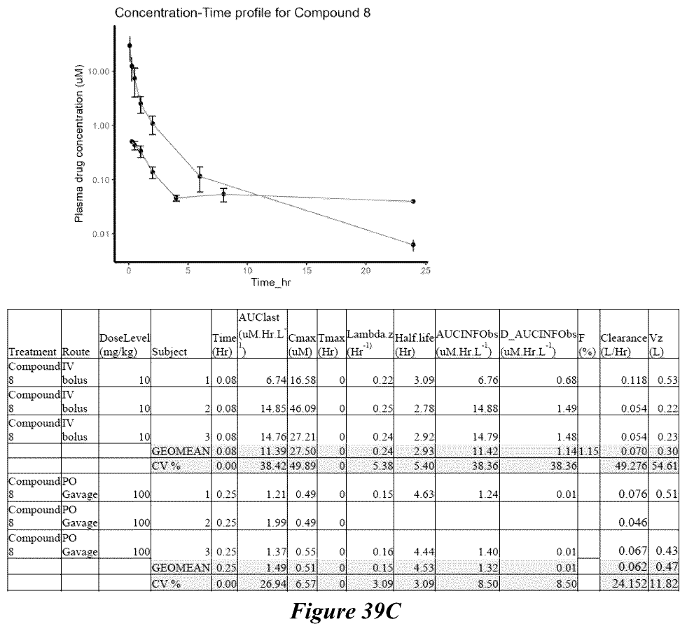

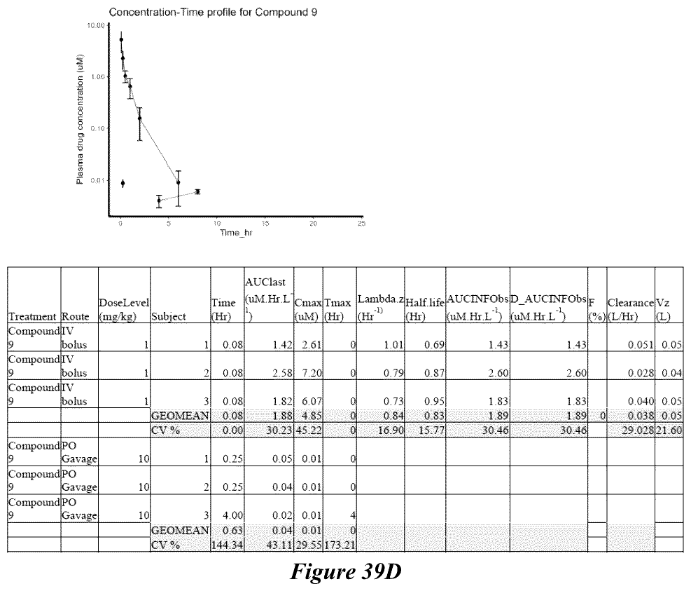

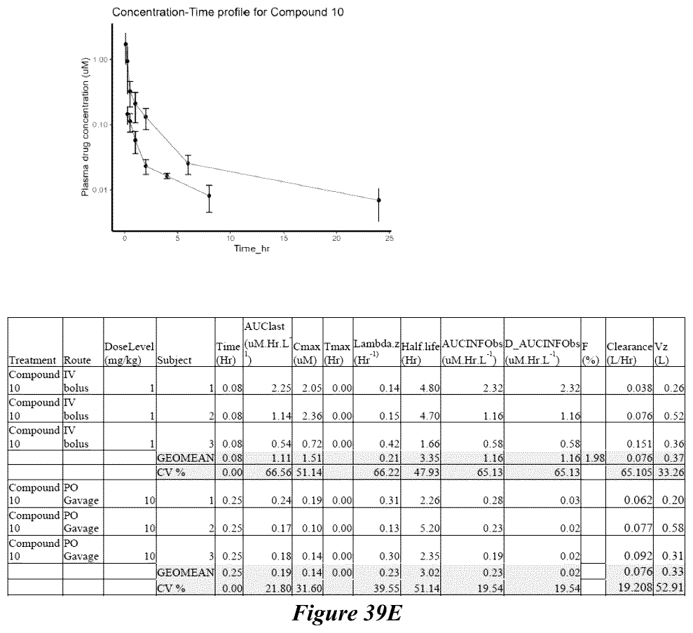

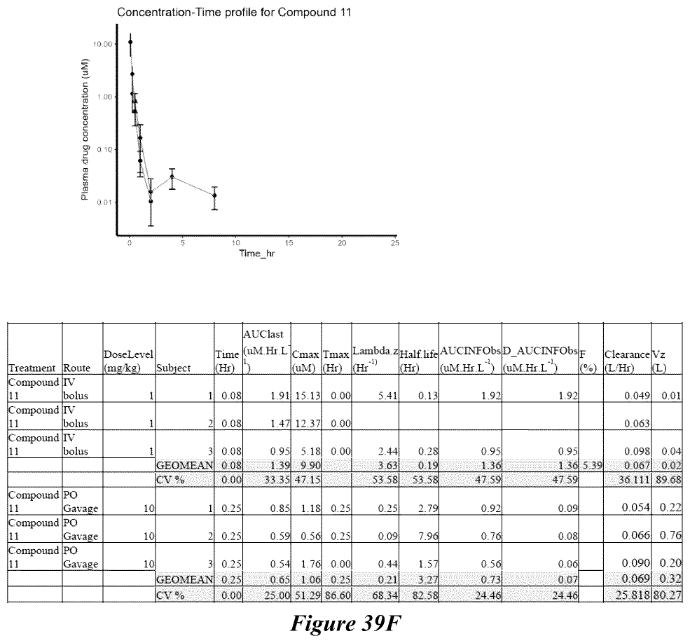

All meta-carborane analogs had elevated systemic IV CL relative to OSU-ERβ-12 (Table 6A) with the 4-carbon alkyl chain analog, Compound 6 (FIG. 39A) having the lowest CL of 0.017 L/hr which is still nearly 3-fold higher than OSU-ERβ-12. Compound 6 was also the only meta-carborane with a longer elimination half-life than OSU-ERβ-12 (11.9 versus 5.4 hrs). Compound 7 (FIG. 39B), the sulfide analog of Compound 6, had a further increase in CL of 7-fold (0.12 L/hr) which was reduced by the inclusion of a terminal —OH to 0.07 L/hr for Compound 8 (FIG. 39C). Oxidation of the sulfide in Compound 8 to a sulfone in Compound 9 (FIG. 39D) was associated with a drop in systemic IV CL to 0.04 L/hr and a reduction in elimination half-life from 2.9 to 0.8 hours. Shortening the carbon tail of Compound 7 by 2 carbons, as in Compound 10 (FIG. 2D, (FIG. 39E)) or 3 carbons to the thiol, as in Compound 11 (FIG. 39F), reduced systemic CL by 5000 in each case, without appreciably impacting elimination half-life. Poor oral absorption and/or extensive first pass metabolism (F %<6) were consistent with rapid IV clearance of the meta series following an oral dose (Table 6B3). Dose adjusted Cₘₐₓ values ranged from 20-fold lower for Compound 11 (1.1 μM) to more than 400-fold lower for Compound 8 (0.5 μM at 100 mg/kg) with similar patterns for AUC_{D_INF} values (179-fold lower [0.07 μM*hr] to 1200-fold lower [0.01 μM*hr] for Compound 11 and Compound 8, respectively).

TABLE 6A
Mouse IV pharmacokinetic parameter estimates for OSU-ERβ-12 and meta-
substituted analogs.

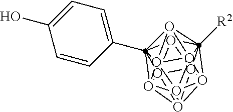

| | | Dose | Clearance, L/hr | V_{d}, L | Elimination T_{1/2}, hr |
|---|---|---|---|---|---|
| Compound | Structure/R² | (mg/kg) | (CV %) | (CV %) | (CV %) |
| OSU-Erβ-12 | 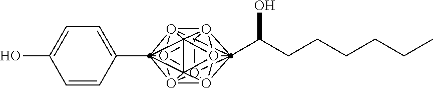 | 10 | 0.006 (16.5) | 0.05 (15.0) | 5.4 (1.35) |

Meta series

| | | | | | |
|---|---|---|---|---|---|
| Compound 6 | 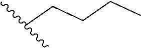 [SMILES: *CCCC] | 1 | 0.017 (28.6) | 0.3 (47.4) | 11.9 (22.62) |
| Compound 7 | 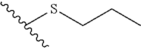 [SMILES: *SCCC] | 1 | 0.12 (59.7) | 0.6 (25.3) | 3.7 (29.9) |
| Compound 8 | 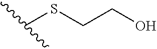 [SMILES: *SCCO] | 10 | 0.07 (49.3) | 0.3 (54.6) | 2.9 (5.4) |
| Compound 9 | 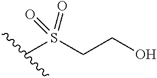 [SMILES: *S(=O)(=O)CCO] | 1 | 0.04 (29.03) | 0.05 (21.6) | 0.8 (15.7) |
| Compound 10 | 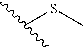 [SMILES: *SC] | 1 | 0.08 (65.1) | 0.37 (33.26) | 3.4 (47.93) |
| Compound 11 | 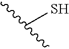 [SMILES: *S] | 1 | 0.07 (36.1) | 0.02 (89.7) | 0.2 (53.58) |

The IV pharmacokinetic characteristics of each compound were determined after single-dose injections in mice followed by serial blood draws. The pharmacokinetic parameter estimates were obtained by non-compartmental analysis.
V_{d}, volume of distribution; T_{1/2}, half-life; CV, coefficient of variation

TABLE 6B
Mouse PO pharmacokinetic parameter estimates for OSU-ERβ-12 and meta-
substituted analogs.

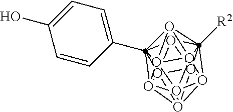

| | | | Cmax, | Tmax, | AUC_{D}_INF, | | Elimination |
|---|---|---|---|---|---|---|---|
| | | Dose | μM | hr | μM*hr | F | T_{1/2}, hr |
| Compound | Structure/R² | (mg/kg) | (CV %) | (CV %) | (CV %) | (%) | (CV %) |
| OSU-Erβ- 12 | 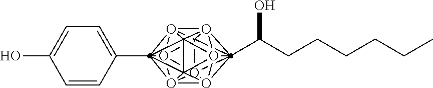 | 10 | 21.8 (15.84) | 0.75 (86.6) | 12.54 (8.21) | 98.9 | 5.4 (7.04) |

Meta series

| | | | | | | | |
|---|---|---|---|---|---|---|---|
| Compound 6 | 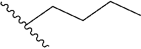 [SMILES: *CCCC] | 10 | 0.2 (42.5) | 0.25 (69.3) | 0.07 (20.38) | 1.3 | 5.2 (56.7) |
| Compound 7 | 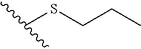 [SMILES: *SCCC] | 10 | 0.2 (28.2) | 0.25 (114.6) | 0.04 (18.24) | 5.5 | 2.1 (13.8) |
| Compound 8 | 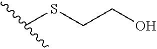 [SMILES: *SCCO] | 100 | 0.5 (6.57) | 0.25 (0.0) | 0.01 (8.5) | 1.2 | 4.5 (3.1) |
| Compound 9 | 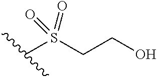 [SMILES: *S(=O)(=O)CCO] | 10 | 0.01 (29.55) | | | | |
| Compound 10 | 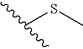 [SMILES: *SC] | 10 | 0.14 (31.6) | 0.25 (0.0) | 0.02 (19.54) | 2 | 3.0 (51.1) |
| Compound 11 | 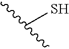 [SMILES: *S] | 10 | 1.1 (51.29) | 0.25 (86.6) | 0.07 (24.46) | 5.4 | 3.3 (82.6) |

The oral pharmacokinetic characteristics of each compound were determined after single-dose oral administration in mice followed by serial blood draws. The pharmacokinetic parameter estimates were obtained by non-compartmental analysis.
Cmax, maximum plasma concentration; Tmax, time of Cmax; AUC_{D}_INF, Dose-adjusted AUC infinity; F, Absolute bioavailability; T_{1/2}, half-life; CV, coefficient of variation

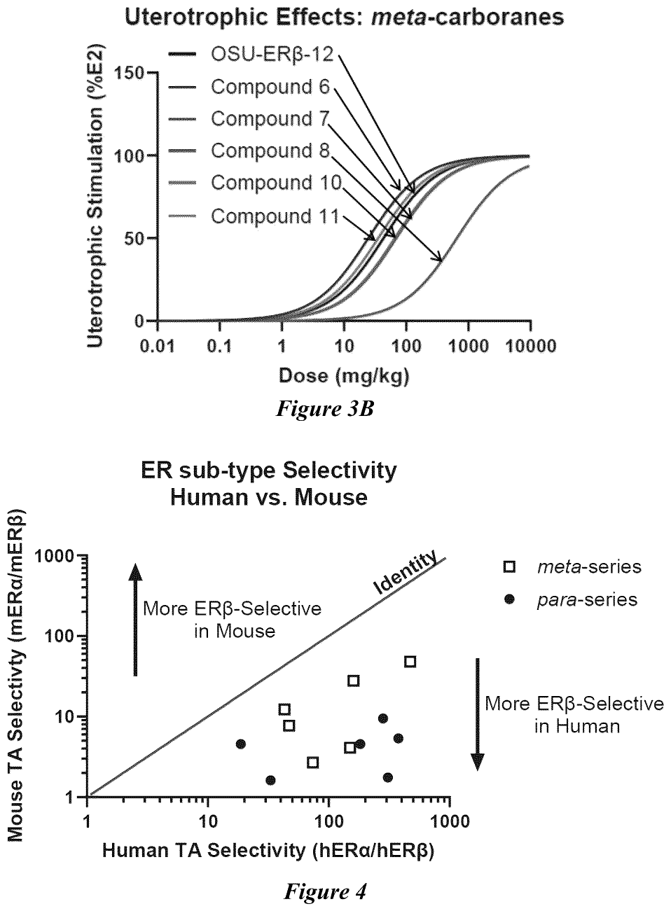

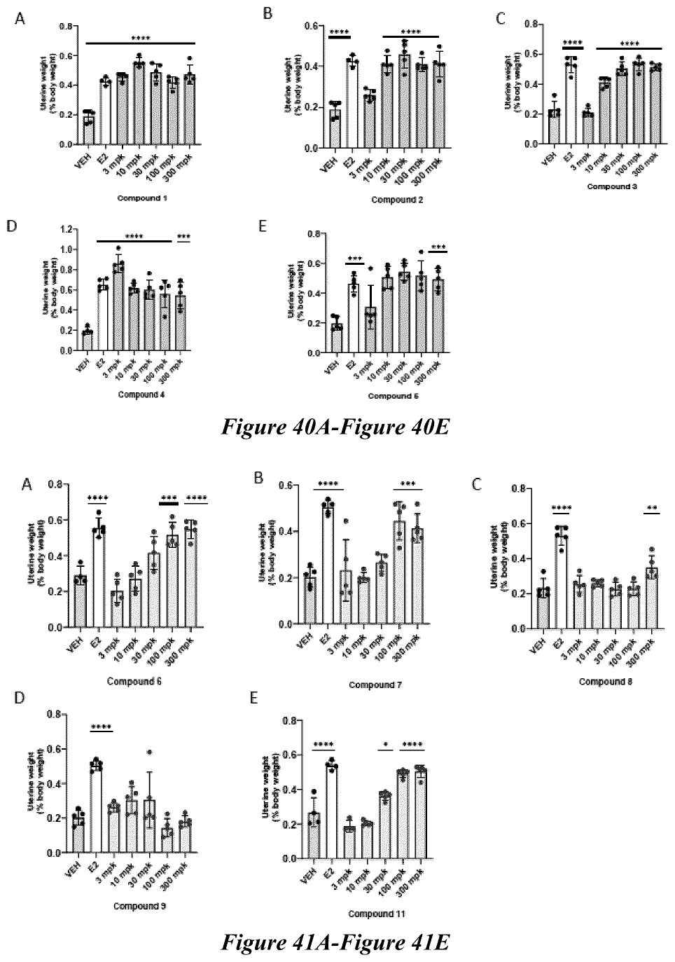

Uterotrophic Studies. To identify dose levels of these analogs that elicit estrogenic responses in vivo, a well-established bioassay of estrogenic uterotrophic stimulation, attributable to ERα, in immature, estrogen-naïve female mice was used (Kleinstreuer et al., 2016). Analogs were administered orally over a dose range to determine the highest dose devoid of ERα-activity, as identified by uterotrophism, and therefore the highest putative ERβ-selective dose. The uterotrophic ED₅₀ of OSU-ERβ-12 was 43.3 mg/kg (Table 7A; FIG. 2E) which was at least 5-fold higher than other para-carborane analogs such as Compound 3 (8.4 mg/kg) and up to 19-fold higher than Compound 5 (2.3 mg/kg). Both Compound 1 and Compound 4 were fully uterotrophic at the lowest dose tested (3 mg/kg) (FIG. 3A-FIG. 3B, FIG. 40A-FIG. 40E), somewhat limiting the ability to compare them to the rest of the para- and meta-carborane series. By contrast, only two meta-carboranes had lower uterotrophic ED₅₀ values compared to that of OSU-ERβ-12, the unsubstituted analog Compound 6 and the thiol analog Compound 11 (Table 7B, 24.6 and 35.5 mg/kg, respectively). The remaining meta-substituted analogs had higher ED₅₀ values including two compounds, Compound 8, which was the least potent in the series, and Compound 9, that had no uterine stimulation at the highest dose tested (300 mg/kg) (FIG. 4, FIG. 41A-FIG. 41E).

Simulated pharmacokinetics at uterotrophic dose levels. To account for large differences in the oral pharmacokinetics among the analogs (Table 5B and Table 6B) in the interpretation of the uterotrophic dose response data, the pharmacokinetics of a uterotrophic ED₅₀ dose were simulated for each analog. These simulated parameters (AUC_{0-24 hr} and Cₘₐₓ) are reported in Table 7A and Table 7B. When correcting for differences in pharmacokinetics among the para-carboranes, the AUC_{0-24 hr} associated with OSU-ERβ-12's uterotrophic stimulation is 316 μM*hr or 316-fold to 29-fold higher than Compound 3 (1 μM*hr) or Compound 2 (11 M*hr), respectively (Table 7A). Likewise, OSU-ERβ-12's Cₘₐₓ of 88.1 μM is 294-fold to 114-fold higher than Compound 5 (0.3 μM) or Compound 2 (0.8 μM), respectively. Similarly, when considering the meta-carboranes, the AUC₀₋₂₄ hr associated with OSU-ERβ-12's uterotrophic stimulation ranged from 226-fold to 102-fold higher than Compound 10 (1.4 μM*hr) or Compound 11 (3.1 μM*hr), respectively (Table 7B). Likewise, OSU-ERβ-12's uterotrophic Cₘₐₓ was 176-fold to 24-fold higher than Compound 6 (0.5 μM) or Compound 11 (3.7 μm), respectively.

In an attempt to account for both known differences in pharmacokinetics among the analogs and differences in hERβ selectivity, the ratio of the simulated uterotrophic Cₘₐₓ and the hERα transactivation EC₅₀ was additionally calculated (last column, Table 7A and Table 7B). The interpretation of this ratio is that values>1 suggest maximum circulating total compound levels exceed those needed to stimulate hERα at uterotrophic dose levels. Amongst the para-carboranes, this analysis predicted only OSU-ERβ-12 and Compound 1 to have sufficient circulating levels to stimulate hERα transactivation at murine uterotrophic doses (ratios of 2.9 and 1. 1, respectively, Table 7A). The remainder of the para-carboranes had ratios ranging from <0.03 (Compound 4) to 0.4 (Compound 3), suggesting that these agents are not predicted to achieve sufficient circulating total compound levels to stimulate hERα. Similarly, none of the meta-carboranes (Table 7B) are predicted to have sufficient circulating levels to stimulate hERα at uterotrophic doses as ratios range from <0.1 (Compound 11) to 0.4 (Compound 7).

TABLE 7A
Simulated pharmacokinetic parameters and predicted hERα activation at uterotrophic
dose for para-substituted analogs.

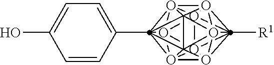

| | | | | | hERα | |
|---|---|---|---|---|---|---|
| | | Uterotrophic | Uterotrophic | | EC₅₀, | (Uterotrophic |
| | | ED₅₀, | ED₅₀ | Uterotrophic | μM | ED₅₀ |
| | | mg/kg | AUC₀₋₂₄ₕᵣ | ED₅₀ Cₘₐₓ | (95% | Cₘₐₓ)/hERα |
| Compound | R¹ | (95% CI) | (μM*hr) | (μM) | CI) | EC₅₀ |
| OSU-Erβ- 12 | 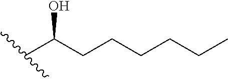 [SMILES: *[C@@H](O)CCCCCC] | 43.3 (34-55) | 316 | 88.1 | >30 | <2.9 |
| Compound 1 | 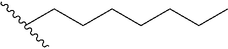 [SMILES: *CCCCCCC] | <3.0 | <23 | <1.3 | 1.1 (0.8- 1.7) | <1.1 |
| Compound 2 | 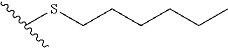 [SMILES: *SCCCCCC] | 3.1 (1.6-5.6) | 11 | 0.8 | 2.7 (1.7- 4.4) | 0.3 |
| Compound 3 | 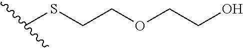 [SMILES: *SCCOCCO] | 8.4 (5.7-12.5) | 1.0 | 0.4 | 1.1 (0.7- 1.7) | 0.4 |
| Compound 4 | 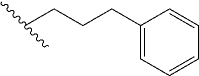 [SMILES: *CCCc1ccccc1] | <3.0 | <14 | 0.8 | >30 | <0.03 |
| Compound 5 | 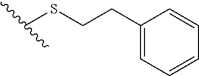 [SMILES: *SCCc1ccccc1] | 2.3 (0.5-5.6) | 2.3 | 0.3 | 2.8 (1.7- 4.6) | 0.1 |

Uterotrophic ED₅₀ represents the dose of the compound required to stimulate 50% of the uterotrophic effects induced by the reference agonist estradiol. For the ratio of Uterotrophic ED₅₀ Cₘₐₓ/hERα EC₅₀, values greater than 1 indicate that, at the uterotrophic dose, the peak plasma concentration exceeds the proposed concentration required to activate ERα.

TABLE 7B
Simulated pharmacokinetic parameters and predicted hERα activation at uterotrophic
dose for OSU-ERβ-12 and meta-substituted analogs

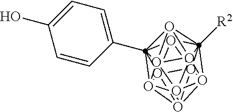

| | | | | | hERα | |
|---|---|---|---|---|---|---|
| | | | | Utero- | EC₅₀, | (Uterotrophic |
| | | Uterotrophic | Uterotrophic | trophic | μM | ED₅₀ |
| | | ED₅₀, mg/kg | ED₅₀ AUC₀₋ | ED₅₀ Cₘₐₓ | (95% | Cₘₐₓ)/hERα |
| Compound | Structure/R² | (95% CI) | ₂₄ₕᵣ (μM*hr) | (μM) | CI) | EC₅₀ |
| OSU-Erβ- 12 | 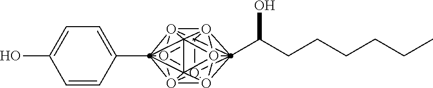 | 43.3 (34-55) | 316 | 88.1 | >30 | <2.9 |

Meta series

| | | | | | | |
|---|---|---|---|---|---|---|
| Compound 6 | 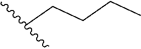 [SMILES: *CCCC] | 24.6 (15.7-38.6) | 1.7 | 0.5 | 2.6 (1.1- 7.2) | 0.2 |
| Compound 7 | 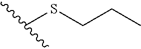 [SMILES: *SCCC] | 69.3 (37.1-133) | 2.4 | 1.5 | 4 (2.2- 8.5) | 0.4 |
| Compound 8 | 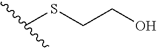 [SMILES: *SCCO] | >300 | >5.2 | >1.5 | 3.9 (2.6- 5.6) | >0.4 |
| Compound 9 | 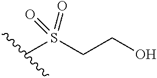 [SMILES: *S(=O)(=O)CCO] | >300 | >1.3 | >0.2 | 5.1 (3.3- 7.8) | >0.04 |
| Compound 10 | 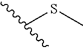 [SMILES: *SC] | 65.7 (47.4-91.4) | 1.4 | 0.9 | 3.1 (16.- 6.5) | 0.3 |
| Compound 11 | 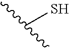 [SMILES: *S] | 35.5 (26.7-47.1) | 3.1 | 3.7 | >30 | <0.1 |

Uterotrophic ED₅₀ represents the dose of the compound required to stimulate 50% of the uterotrophic effects induced by the reference agonist estradiol. For the ratio of Uterotrophic ED₅₀ Cₘₐₓ/hERα EC₅₀, values greater than 1 indicate that, at the uterotrophic dose, the peak plasma concentration exceeds the proposed concentration required to activate ERα.

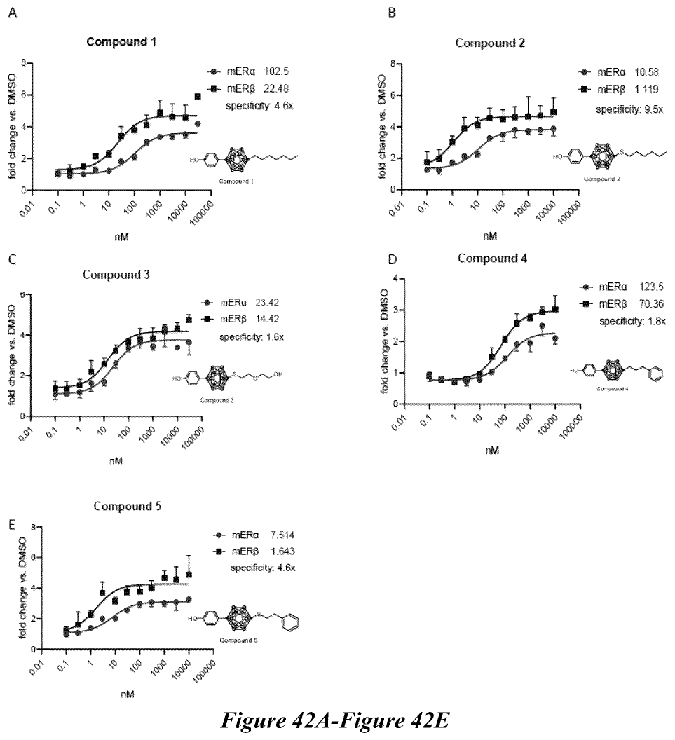

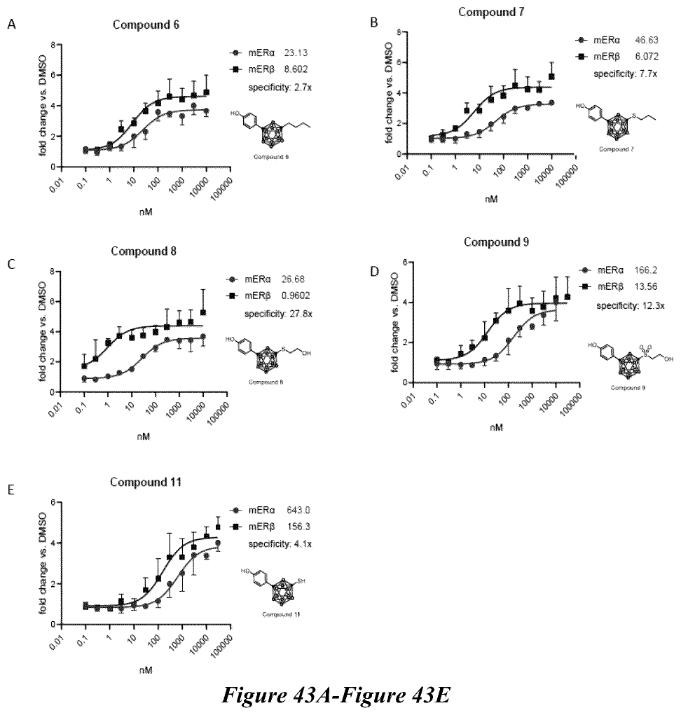

Murine ER α/β transactivation. To better understand why the majority of the analogs demonstrated uterotrophic activity in estrogen naïve female mice at dose levels that were, in many cases, well below those predicted to activate hERα, the transactivation assays were repeated with murine versions of each ER subtype (mERα and mERβ, Table 8A-Table 8B, FIG. 2G-FIG. 2H; FIG. 42A-FIG. 42E and FIG. 43A-FIG. 43E). OSU-ERβ-12 remained ERβ-selective (5.4-fold versus mERα) in the murine transactivation assay with an EC₅₀ 52.2 nM for mERβ (Table 8A). Except for Compound 4 (EC₅₀ for ERβ, 70.4 nM), all of the other para-carboranes tested were more potent in mERβ transactivation than OSU-ERβ-12 with EC₅₀ values ranging from 1.1 nM (Compound 2) to 22.5 nM (Compound 1). Compound 2 was also more selective for mERβ (9.5-fold over mERα) than OSU-ERβ-12 but all other para-carborane analogs demonstrated reduced mERβ-selectivity ranging from 1.6-fold (Compound 3) to 4.6-fold (Compound 1 and Compound 5). In general, the para-carborane series was more potent and less ERβ-selective in murine transactivation assays when compared to human, while also being less sensitive to structural modifications. With the exception of Compound 11 (EC₅₀ 156 nM), each of the meta-carboranes tested was also more potent in mERβ transactivation than OSU-ERβ-12 (Table 8B), with EC₅₀ values ranging 1 nM (Compound 8) to 14 nM (Compound 9). Selectivity for mERβ over mERα varied widely in the meta-carborane series with most analogs demonstrating greater selectivity than OSU-ERβ-12, ranging from 7.7-fold (Compound 7) to 48-fold (Compound 10; FIG. 2H). Only Compound 11 (4.1-fold) and Compound 6 (2.7-fold) were less selective. As with the para-carborane series, the meta-carboranes were more potent and less ERβ-selective in murine transactivation assays when compared to human, but activity profiles varied widely by structure.

Given the lack of concordance in the structure-activity relationship of both the meta- and para-series between human and mouse transactivation assays, the analysis of the ratio of the projected Cₘₐₓ at a uterotrophic dose and the transactivation EC₅₀ in mERα was repeated (Table 8A and Table 8B, last column). For each compound, across both series, the projected Cₘₐₓ exceeded the mERα transactivation EC₅₀ (all values>1).

TABLE 8A
Mouse ERα/β transactivation activities and predicted mERα activation at uterotrophic
dose of para-substituted analogs.

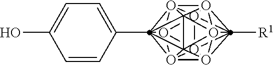

| | | | Transactivation^{b} | | | |
|---|---|---|---|---|---|---|
| | | | mERα | mERβ | | |
| | | | EC₅₀, | EC₅₀, | | (Uterotrophic |
| | | Uterotrophic | nM | nM | | EC₅₀ |
| | | ED₅₀ Cₘₐₓ^{a} | (95% | (95% | | Cₘₐₓ)/mERα |
| Compound | R¹ | (μM) | CI) | CI) | Selectivity | EC₅₀^{c} |
| OSU-ERβ- 12 | 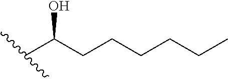 [SMILES: *[C@@H](O)CCCCCC] | 88.1 | 280 (103.8- 720.7) | 52.2 (27.2- 102.6) | 5.4 | 615.1 |
| Compound 1 | 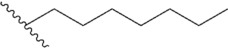 [SMILES: *CCCCCCC] | <1.3 | 102.5 (70.7- 147.6) | 22.5 (10.2- 50.2) | 4.6 | <12.3 |
| Compound 2 | 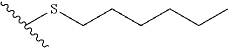 [SMILES: *SCCCCCC] | 0.8 | 10.6 (6.0- 18.3) | 1.1 (0.37- 3.4) | 9.5 | 72.8 |
| Compound 3 | 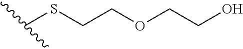 [SMILES: *SCCOCCO] | 0.4 | 23.4 (12.4- 43.9) | 14.4 (7.0- 30.2) | 1.6 | 17.5 |
| Compound 4 | 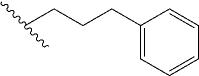 [SMILES: *CCCc1ccccc1] | <0.8 | 123.5 (70.8- 216.5) | 70.4 (48.6- 102) | 1.8 | <6.8 |
| Compound 5 | 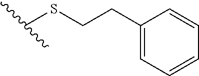 [SMILES: *SCCc1ccccc1] | 0.3 | 7.5 (3.3- 16.8) | 6 (0.5-7.7) | 4.6 | 39.9 |

^{a}Uterotrophic ED₅₀ represents the dose of the compound required to stimulate 50% of the uterotrophic effects induced by the reference agonist, estradiol.
^{b}Each compound was tested for mouse ERα and ERβ agonism in a luciferase transactivation assay. The EC₅₀ values are shown in nanomolar. Transactivation assay selectivity was calculated as a ratio of the EC₅₀ for mERα/mERβ.
^{c}For the ratio of Uterotrophic ED₅₀ Cₘₐₓ/mERα EC₅₀, values greater than 1 indicate that, at the uterotrophic dose, the peak plasma concentration exceeds the proposed concentration required to activate mouse ERα.

TABLE 8B
Mouse ERα/β transactivation activities and predicted mERα activation at uterotrophic
dose of OSU-ERβ-12 and meta-substituted analogs

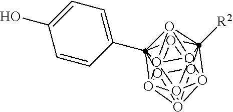

| | | | Transactivation^{b} | | | |
|---|---|---|---|---|---|---|
| | | | mERα | mERβ | | |
| | | | EC₅₀, | EC₅₀, | | (Uterotrophic |
| | | Uterotrophic | nM | nM | | dose |
| | | dose EC₅₀ Cₘₐₓ^{a} | (95% | (95% | | Cₘₐₓ)/mERα |
| Compound | Structure/R² | (μM) | CI) | CI) | Selectivity | EC₅₀^{c} |
| OSU-ERβ- 12 | 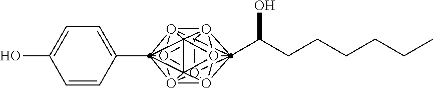 | 88.1 | 280 (102-721) | 52.2 (27.2- 103) | 5.4 | 315.1 |

Meta series

| | | | | | | |
|---|---|---|---|---|---|---|
| Compound 6 | 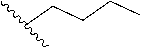 [SMILES: *CCCC] | 0.5 | 23.1 (11.3- 46.8) | 8.6 (3.4- 21.6) | 2.7 | 21.6 |
| Compound 7 | 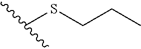 [SMILES: *SCCC] | 1.5 | 46.6 (26.9- 80.8) | 6 (1.8- 21.9) | 7.7 | 32.6 |
| Compound 8 | 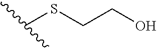 [SMILES: *SCCO] | 1.5 | 26.7 (15.9- 44.7) | 1 (0.1- 29.2) | 27.8 | 57 |
| Compound 9 | 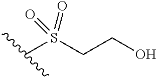 [SMILES: *S(=O)(=O)CCO] | 0.2 | 166.2 (75-392) | 13.7 (4.9- 36.3) | 12.3 | 1.3 |
| Compound 10 | 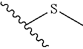 [SMILES: *SC] | 0.9 | 29.5 (12.2- 69.1) | 0.6 (0.07- 3.8) | 47.9 | 21.3 |
| Compound 11 | 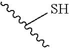 [SMILES: *S] | 3.7 | 643 (328- 1261) | 156 (67-375) | 4.1 | 5.8 |

^{a}Uterotrophic ED₅₀ represents the dose of the compound required to stimulate 50% of the uterotrophic effects induced by the reference agonist, estradiol.
^{b}Each compound was tested for mouse ERα and ERβ agonism in a luciferase transactivation assay. The EC₅₀ values are shown in nanomolar. Transactivation assay selectivity was calculated as a ratio of the EC₅₀ for mERα/mERβ.
^{c}For the ratio of Uterotrophic ED₅₀ Cₘₐₓ/mERα EC₅₀, values greater than 1 indicate that, at the uterotrophic dose, the peak plasma concentration exceeds the proposed concentration required to activate mouse ERα

Discussion. SERMs have a firmly established role in the management of many diseases related to estrogen signaling [1,14]. Interest in improving therapeutic estrogen pharmacology remains high as the understanding of endocrine disease mechanisms, and thus the potential utility of estrogen therapy, expands (An, 2016; Goldstein, 2022; Li et al., 2016; Liu, 2020; Patel & Bihani, 2018; Pollock & Parker, 2022). ERβ-selective carborane-based estrogens have already demonstrated their potential application in the treatment of several hormone-dependent diseases (Banerjee et al., 2022; Datta et al., 2022; Sharma et al., 2022). This report builds on the findings from an earlier in vitro structure-activity relationship study of para-carborane ERβ-selective agonists (Sedlák et al., 2021) with additional para- and meta-carborane analogs in the pursuit of increased ERβ potency and selectivity over ERα. The use of carborane pharmacophores in medicinal chemistry to elicit high affinity ligand-receptor interactions is uncommon but a growing number of research groups are exploring carboranes to target diverse proteins of therapeutic interest (Marfavi et al., 2022). To date, work with carborane-based pharmacophores has been largely limited to cell-free and in vitro systems such that in-depth characterization of in vivo carborane pharmacology has never been reported. To this end, in vitro receptor binding and activation studies were expanded upon with full characterization of the murine pharmacokinetics of a carborane pharmacophore and an accounting of in vivo estrogen pharmacology in immature, estrogen naïve female mice.

Of the para-carborane analogs tested, only Compound 3 improved upon OSU-ERβ-12's cell-free binding to hERβ, and only Compound 5 demonstrated a modest improvement in binding selectivity to hERβ over hERα. Most of the para-carboranes displayed more potent stimulation of hERβ transactivation, which was, however, accompanied by more potent stimulation of hERα, and thus reduced functional hERβ selectivity. It is interesting to note that the meta series of analogs were more selective in their cell-free binding to hERβ than the lead OSU-ERβ-12, however, this did not translate to functional activity at the receptor. Apart from Compound 10, each of the meta series analogs was less selective for ERβ transactivation (Table 4B). Similarly, ERβ binding potency did not readily translate to potency in ERβ transactivation. For example, Compound 10, the compound with the highest ERβ transactivation potency, (EC₅₀ of 6.6 nM) demonstrated nearly 4- to 12-fold lower ERβ binding affinity (5.9 nM) relative to the most potent ERβ binding analogs (Compound 8 and Compound 9). However, the in vitro functional activity of the analogs was assessed following transient transfection of either ER subtype into ER negative HEK-293 cells, resulting in non-physiological levels of ER expression, which likely obscures subtle differences in ligand-dependent ER-mediated transcription. When this limitation is considered along with the likelihood that most estrogen responsive tissues contain cells expressing both ER subtypes, these results should be interpreted with caution when projecting to in vivo activity.

Comparisons of the murine pharmacokinetic properties of the para and meta-substituted analogs with the lead ERβ agonist, OSU-ERβ-12 revealed that, among the para-carboranes, only Compound 3 departed substantially from the IV pharmacokinetics of OSU-ERβ-12, with the majority of analogs having low clearance rates and longer elimination half-lives. As a series, the meta-carboranes exhibited both elevated systemic clearance and reduced elimination half-lives following an IV dose. Collectively, the analogs in both series had poorer pharmacokinetic characteristics than OSU-ERβ-12 following a single oral dose (F % ranging from 1 to 43%, Table 5A-Table 6B). This finding highlights the importance of the sites of the sulfur and oxygen substitutions on the alkyl chain as they pertain to metabolic stability and systemic exposure following an oral dose. For example, the terminal hydroxyl group of Compound 3, Compound 8 and Compound 9 increased IV systemic clearance substantially compared to close analogs without terminal hydroxyls (Compound 2 and Compound 7). Likewise, the sulfide compounds Compound 2, Compound 5, and Compound 7, had higher IV systemic clearance compared to their carbon counterparts, Compound 1, Compound 4, and Compound 6, respectively. Only one analog, Compound 1, which differs from OSU-ERβ-12 by its absence of a chiral hydroxyl moiety, achieved even 50% of the systemic exposure resulting from an oral dose of OSU-ERβ-12. However, Compound 1's 4-fold reduction in Cₘₐₓ and its substantially lower bioavailability (F %=41.3) suggests this compound's absorption may be limiting and the added polarity provided by OSU-ERβ-12's chiral —OH is an important feature of its exceptional oral pharmacokinetics in mice. Mouse pharmacokinetic data for the relevant clinical comparators ERB-041 or LY500307 have not been published. However, limited rat pharmacokinetic data for LY500307 suggest 10 mg/kg oral doses are associated with 517-1,299 ng*hr/ml or 1.8-4.6 M*hr AUC_{0-24 hrs} which is ˜25-fold lower than the corresponding AUCₗₐₛₜ following a 10 mg/kg oral dose for OSU-ERβ-12 in mice (120.72 μM*hr) (Hilbish et al., 2013). By this means of indirect comparison, all but one meta-carborane (Compound 3) demonstrated improved oral rodent pharmacokinetics compared to LY500307. Furthermore, OSU-ERβ-12 displays improved ERβ potency (79 vs. 234 nM EC₅₀, respectively), selectivity over ERα (>376 vs. 40-fold, respectively) and reduced systemic clearance following an IV dose in mice (6 mL/hr vs. 700 mL/hr, respectively) when compared to the recently published ERβ-selective agonist CIDD-0149897 which has reported anti-glioblastoma effects (Pratap et al., 2023) similar to OSU-ERβ-12 (Sharma et al., 2022). When taken together, these data suggest OSU-ERβ-12 represents a compelling therapeutic estrogen.

Uterotrophic effects of estrogens are well characterized and completely ascribed to ERα as opposed to ERβ stimulation (Yan et al., 2006). It was therefore surprising to observe that some analogs, especially within the para series, were more potent on a mg/kg basis than OSU-ERβ-12 at inducing uterotrophic effects in estrogen naïve female mice despite their poor pharmacokinetic profile and high selectivity for ERβ (FIG. 3A and FIG. 3B). It is important to highlight that dosing regimens of highly ERα selective ligands, such as PPT (Harris, Katzenellenbogen, et al., 2002), have been reported that result in beneficial estrogen pharmacology absent overt uterotrophic effects (Bolego et al., 2010) in mice. The existence of such regimens suggests that well accepted assays for uterotrophism, like those utilized herein, do not necessarily constitute definitive evidence of in vivo ERβ selectivity. Nevertheless, it was hypothesized that a possible explanation for the discrepancy between the in vitro and in vivo activity data is that the initial selectivity data were generated in cell systems expressing hERα or β whereas the uterotrophic studies were conducted in mice. After conducting similar experiments using cell systems expressing mER α or β (Table 8A and Table 8B), it was observed that potency in mERα transactivation was increased over that of hERα, and ERβ selectivity was reduced for every compound (FIG. 4). The most ERβ selective agonist in mouse (48-fold, Compound 10, Table 8B) was 473-fold ERβ selective in human assays (Table 4B). Likewise, some highly selective compounds in human assays (Compound 4, >102.8-fold ERβ-selective, Table 4B), were essentially non-selective in mouse (1.75-fold ERβ-selective, Table 8A). The data clearly highlight differences in the ability of these carborane subtype selective agonists to activate hERs versus mERs as likely confounding the interpretation of the uterotrophic dose response data.

There is less than 90% sequence homology between human and murine ERα/β and species specific ER binding of novel therapeutic estrogens has been previously reported (Harris, Bapat, et al., 2002). In human, the ligand binding domain (LBD) of ERα differs from ERβ by only two amino acids which are thought to interact with bound ligands (Manas et al., 2004). There are no published crystal structures of mERα and mERβ, but homology models support similar modes of binding for several ligands between mERα and hERα (Gonzalez et al., 2019). A simple sequence alignment of human and mouse ERα and ERβ also suggest similar differences in residues interacting with ligands in the LBD may afford mERβ selectivity over mERα (data not shown). Therefore, the apparent differences in mouse activity are unlikely due to large differences in binding conformation between the species, but instead are likely the result of differential transcriptional co-regulator interactions subsequent to ligand binding that drive fine-tuned ER-mediated transcription (Green & Carroll, 2007). To account for this, an improved murine assay system would include murine cells over expressing murine receptor.

To further understand the unexpected potent uterotrophic effects, simulation analyses were conducted to explore the possibility of species-specific differences in ER pharmacology. The PK profile of each analog at its uterotrophic EC₅₀ dose was simulated and compared with the human and mouse ERα EC₅₀ for transcriptional activation. In most cases, the Cₘₐₓ from the simulated pharmacokinetic curves at a uterotrophic dose exceeded mouse, but not human, ERα EC₅₀ (Table 7A-Table 7B and Table 8A-Table 8B). It is appreciated that differences in plasma protein binding among the analogs are likely given the effects of some of the substitutions on polarity and are expected to impact the uterotrophic effects. Nevertheless, the simplistic pharmacokinetic simulations substantiated the species differences in ER pharmacology and helped explained the observed uterotrophic effects of the analogs in estrogen naïve female mice.

The majority of considerations given to differences between human and preclinical species in small molecule drug development focus on drug disposition and how it may in turn impact pharmacodynamics (Namdari et al., 2021). There is renewed focus on the importance of species dependent drug-target interactions with mAb therapeutics given their exquisite target specificity (Mould & Meibohm, 2016), however few reports on small molecules consider species-specific drug action at target receptors. This data shows that pharmacokinetic data when combined with robust in vitro and in vivo pharmacology assays can be used to define species-specific receptor pharmacology and deconvolute complex structure activity relationships. Analyses such as that herein occur frequently within industrial drug development programs but seldom, if ever, are published. As such, the analysis using a series of subtype selective para- and meta-carborane estrogen receptor agonists is provided herein as a case study in the elucidation of species-specific in vivo receptor pharmacology.

In conclusion, these studies highlight the species-specific estrogen receptor pharmacology of subtype selective para- and meta-carboranes and the challenges in characterizing new therapeutic estrogens for potential human use in preclinical species like the mouse. The limitations of the in vitro affinity and activity assays to model the complexity of estrogen signaling within tissues are recognized, which is to say nothing of the well-recognized variant splice forms of ERα or ERβ that make modelling composite estrogen pharmacology even more challenging (Warner et al., 2021). Nevertheless, the data shows that carborane-based subtype selective estrogen receptor agonists like OSU-ERβ-12 have activity profiles and pharmacokinetics consistent with a potential therapeutic and represent an exciting new class of therapeutic estrogens.

### REFERENCES

An, K. C. (2016). Selective Estrogen Receptor Modulators. Asian Spine J, 10(4), 787-791. https://doi.org/10.4184/asj.2016.10.4.787
Archer, D. F. (2011). The gynecologic effects of lasofoxifene, an estrogen agonist/antagonist, in postmenopausal women. Menopause, 18(1), 6-7. https://doi.org/10.1097/gme.0b013e318203a46b
Asnake, S. et al. (2019). Species differences in ligand interaction and activation of estrogen receptors in fish and human. J Steroid Biochem Mol Biol, 195, 105450. https://doi.org/10.1016/j.jsbmb.2019.105450
Banerjee, A. et al. (2022). A Novel Estrogen Receptor R Agonist Diminishes Ovarian Cancer Stem Cells via Suppressing the Epithelial-to-Mesenchymal Transition. Cancers (Basel), 14(9). https://doi.org/10.3390/cancers14092311
Bingham, B. et al. (2007). Species-specific in vitro pharmacological effects of the cannabinoid receptor 2 (CB2) selective ligand AM1241 and its resolved enantiomers. Br J Pharmacol, 151(7), 1061-1070. https://doi.org/10.1038/sj.bjp.0707303
Bolego, C. et al. (2010). Selective estrogen receptor-alpha agonist provides widespread heart and vascular protection with enhanced endothelial progenitor cell mobilization in the absence of uterotrophic action. Faseb j, 24(7), 2262-2272. https://doi.org/i0.1096/fj.09-139220
Cheng, Y., & Prusoff, W. H. (1973). Relationship between the inhibition constant (K1) and the concentration of inhibitor which causes 50 percent inhibition (I50) of an enzymatic reaction. Biochem Pharmacol, 22(23), 3099-3108. https://doi.org/10.1016/0006-2952(73)90196-2
Cleuren, A. C. et al. (2010). 17α-Ethinylestradiol rapidly alters transcript levels of murine coagulation genes via estrogen receptor α. J Thromb Haemost, 8(8), 1838-1846. https://doi.org/10.1111/j.1538-7836.2010.03930.x
Datta, J. et al. (2022). Activity of Estrogen Receptor β Agonists in Therapy-Resistant Estrogen Receptor-Positive Breast Cancer. Front Oncol, 12, 857590. https://doi.org/10.3389/fonc.2022.857590
Davies, B., & Morris, T. (1993). Physiological parameters in laboratory animals and humans. Pharm Res, 10(7), 1093-1095. https://doi.org/10.1023/a:1018943613122
Davies, M. et al. (2020). Improving the Accuracy of Predicted Human Pharmacokinetics: Lessons Learned from the AstraZeneca Drug Pipeline Over Two Decades. Trends Pharmacol Sci, 41(6), 390-408. https://doi.org/10.1016/j.tips.2020.03.004
Dupont, S. et al. (2000). Effect of single and compound knockouts of estrogen receptors alpha (ERalpha) and beta (ERbeta) on mouse reproductive phenotypes. Development, 127(19), 4277-4291. https://doi.org/10.1242/dev.127.19.4277
Farzaneh, S., & Zarghi, A. (2016). Estrogen Receptor Ligands: A Review (2013-2015). Sci Pharm, 84(3), 409-427. https://doi.org/10.3390/scipharm84030409
Goldstein, S. R. (2022). Selective estrogen receptor modulators and bone health. Climacteric, 25(1), 56-59. https://doi.org/10.1080/13697137.2021.1936485
Gonzalez, T. L. et al. (2019). Homology models of mouse and rat estrogen receptor-α ligand-binding domain created by in silico mutagenesis of a human template: molecular docking with 17β-estradiol, diethylstilbestrol, and paraben analogs. Comput Toxicol, 10, 1-16. https://doi.org/10.1016/j.comtox.2018.11.003
Grant, C. V. et al. (2022). A novel targeted approach to delineate a role for estrogen receptor-β in ameliorating murine mammary tumor-associated neuroinflammation. Endocrine, 75(3), 949-958. https://doi.org/10.1007/si2020-021-02931-7
Green, K. A., & Carroll, J. S. (2007). Oestrogen-receptor-mediated transcription and the influence of cofactors and chromatin state. Nat Rev Cancer, 7(9), 713-722. https://doi.org/10.1038/nrc2211
Harris, H. A., Bapat, A. R. et al. (2002). The ligand binding profiles of estrogen receptors alpha and beta are species dependent. Steroids, 67(5), 379-384. https://doi.org/10.1016/s0039-128x(01)00194-5
Harris, H. A., Katzenellenbogen, J. A. et al. (2002). Characterization of the biological roles of the estrogen receptors, ERalpha and ERbeta, in estrogen target tissues in vivo through the use of an ERalpha-selective ligand. Endocrinology, 143(11), 4172-4177. https://doi.org/10.1210/en.2002-220403
Heldring, N. et al. (2007). Estrogen receptors: how do they signal and what are their targets. Physiol Rev, 87(3), 905-931. https://doi.org/10.1152/physrev.00026.2006
Helms, T. et al. (2020). Selective estrogen receptor modulator, OSU-ERB-12, ameliorates preclinical models of hepatic fibrosis and nash. Hepatology, 72(number1), 1035-1036.
Hewitt, S. C., & Korach, K. S. (2018). Estrogen Receptors: New Directions in the New Millennium. Endocr Rev, 39(5), 664-675. https://doi.org/10.1210/er.2018-00087
Hilbish, K. G., et al. (2013). Fertility and developmental toxicity assessment in rats and rabbits with LY500307, a selective estrogen receptor beta (ERβ) agonist. Birth Defects Res B Dev Reprod Toxicol, 98(5), 400-415. https://doi.org/10.1002/bdrb.21083
Jordan, V. C. (2001). Selective estrogen receptor modulation: a personal perspective. Cancer Res, 61(15), 5683-5687.
Jordan, V. C., & Brodie, A. M. H. (2007). Development and evolution of therapies targeted to the estrogen receptor for the treatment and prevention of breast cancer. Steroids, 72(1), 7-25. https://doi.org/https://doi.org/10.1016/j.steroids.2006.10.009
Kleinstreuer, N. C. et al. (2016). A Curated Database of Rodent Uterotrophic Bioactivity. Environ Health Perspect, 124(5), 556-562. https://doi.org/10.1289/ehp.1510183
Kumar, P. et al. (2023). SAT005 Effect Of OSU-ERβ-12, A Carborane-based ERβ-selective Agonist, On Hepatic Fibrosis And NASH. J Endocr Soc. 2023 Oct. 5; 7(Suppl 1):bvad114.1743. doi: 10.1210/jendso/bvad114.1743.
Li, F. et al. (2016). The selective estrogen receptor modulators in breast cancer prevention. Cancer Chemother Pharmacol, 77(5), 895-903. https://doi.org/10.1007/s00280-016-2959-0
Liu, J. H. (2020). Selective estrogen receptor modulators (SERMS): keys to understanding their function. Menopause, 27(10), 1171-1176. https://doi.org/10.1097/gme.0000000000001585
Manas, E. S. et al. (2004). Structure-based design of estrogen receptor-beta selective ligands. J Am Chem Soc, 126(46), 15106-15119. https://doi.org/10.1021/ja047633o
Marfavi, A., Kavianpour, P., & Rendina, L. M. (2022). Carboranes in drug discovery, chemical biology and molecular imaging. Nature Reviews Chemistry, 6(7), 486-504. https://doi.org/10.1038/s41570-022-00400-x
Mohler, M. L. et al. (2010). Estrogen receptor beta selective nonsteroidal estrogens: seeking clinical indications. Expert Opin Ther Pat, 20(4), 507-534. https://doi.org/10.1517/13543771003657164
Mould, D. R., & Meibohm, B. (2016). Drug Development of Therapeutic Monoclonal Antibodies. BioDrugs, 30(4), 275-293. https://doi.org/10.1007/s40259-016-0181-6
Namdari, R. et al. (2021). Species selection for nonclinical safety assessment of drug candidates: Examples of current industry practice. Regul Toxicol Pharmacol, 126, 105029. https://doi.org/10.1016/j.yrtph.2021.105029
OECD. (2007). Test No. 440: Uterotrophic Bioassay in Rodents. https://doi.org/doi:https://doi.org/i0.1787/9789264067417-en
Ohta, K. et al. (2017). Design and synthesis of iodocarborane-containing ligands with high affinity and selectivity toward ERβ. Bioorg Med Chem Lett, 27(17), 4030-4033. https://doi.org/10.1016/j.bmcl.2017.07.053
Patel, H. K., & Bihani, T. (2018). Selective estrogen receptor modulators (SERMs) and selective estrogen receptor degraders (SERDs) in cancer treatment. Pharmacol Ther, 186, 1-24. https://doi.org/10.1016/j.pharmthera.2017.12.012
Paterni, I. et al. (2014). Estrogen receptors alpha (ERα) and beta (ERβ): subtype-selective ligands and clinical potential. Steroids, 90, 13-29. https://doi.org/10.1016/j.steroids.2014.06.012
Pollock, J. A., & Parker, H. K. (2022). Advances in the Development of Prodrugs as Selective Modulators of Estrogen Receptors. J Endocr Soc, 6(12), bvac158. https://doi.org/10.1210/jendso/bvac158
Pratap, U. P. et al. (2023). Preclinical Development of Brain Permeable ERβ Agonist for the Treatment of Glioblastoma. Mol Cancer Ther, 22(11), 1248-1260. https://doi.org/10.1158/1535-7163.Mct-23-0031
Riggs, B. L., & Hartmann, L. C. (2003). Selective estrogen-receptor modulators—mechanisms of action and application to clinical practice. N Engl J Med, 348(7), 618-629. https://doi.org/10.1056/NEJMra022219
Roehrborn, C. G. et al. (2015). Estrogen receptor beta agonist LY500307 fails to improve symptoms in men with enlarged prostate secondary to benign prostatic hypertrophy. Prostate Cancer Prostatic Dis, 18(1), 43-48. https://doi.org/10.1038/pcan.2014.43
Roman-Blas, J. A. et al. (2010). Efficacy and safety of a selective estrogen receptor R agonist, ERB-041, in patients with rheumatoid arthritis: a 12-week, randomized, placebo-controlled, phase II study. Arthritis Care Res (Hoboken), 62(11), 1588-1593. https://doi.org/10.1002/acr.20275
Rosenzweig, R. et al. (2022). Estrogen Receptor-β Agonists Modulate T-Lymphocyte Activation and Ameliorate Left Ventricular Remodeling During Chronic Heart Failure. Circ Heart Fail, 15(7), e008997. https://doi.org/10.1161/circheartfailure.121.008997
Sedlák, D. et al. (2021). Structure-Activity Relationship of para-Carborane Selective Estrogen Receptor R Agonists. J Med Chem, 64(13), 9330-9353. https://doi.org/10.1021/acs.jmedchem.1c00555
Sharma, P. et al. (2022). Preclinical characterization of OSU-ERb-12, a novel ERβ agonist in Glioblastoma. Cancer Research, 82(12_Supplement), 1867-1867.
Swaby, R. F. et al. (2007). SERMs for the treatment and prevention of breast cancer. Rev Endocr Metab Disord, 8(3), 229-239. https://doi.org/10.1007/s11154-007-9034-4
Thakur, A. et al. (2024). Sex and the Kidney Drug-Metabolizing Enzymes and Transporters: Are Preclinical Drug Disposition Data Translatable to Humans? Clin Pharmacol Ther. https://doi.org/10.1002/cpt.3277
Warner, M. et al. (2021). 25 years of ERβ: a personal journey. J Mol Endocrinol, 68(1), R1-r9. https://doi.org/10.1530/jme-21-0121
Warner, M. et al. (2020). Ventral prostate and mammary gland phenotype in mice with complete deletion of the ERβ gene. Proc Natl Acad Sci USA, 117(9), 4902-4909. https://doi.org/10.1073/pnas.1920478117
Yan, W. et al. (2006). Uterotrophic effects of relaxin related to age and estrogen receptor activation in neonatal pigs. Reproduction, 131(5), 943-950. https://doi.org/10.1530/rep.1.00762
Zidon, T. M. et al. (2020). Effects of ERβ and ERα on OVXinduced changes in adiposity and insulin resistance. J Endocrinol, 245(1), 165-178. https://doi.org/10.1530/joe-19-0321

### List of Abbreviations

ER—Estrogen Receptor
SERM—Selective Estrogen Receptor Modulator
LLOQ—Lower Limit of Quantification
DMSO—Dimethyl Sulfoxide
DMEM—Dulbecco's Modified Eagle Medium

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1.-22. (canceled)

23. A compound defined by Formula XI, or a pharmaceutically acceptable salt thereof 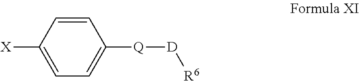
wherein
Q is a substituted or unsubstituted dicarba-closo-dodecaborane cluster, and 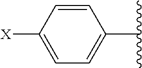
and D are attached to Q in a meta configuration;
D is —S—, —S(O)—, —S(O)(O)—, —S(O)(NH)—, —P(O)(OH)O—, —P(O)(OH)NH—, or —O—;
X is OH, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, or substituted or unsubstituted C₄-C₂₀ alkylheterocycloalkyl; and
R² is H, OH, halogen, or substituted or unsubstituted C₁-C₄ alkyl.

24. The compound of claim 23, wherein Q is 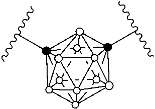
wherein
● is a carbon atom or a boron atom; and
◯ is C—H, C-halogen, C-alkyl, C—OH, C—NH₂, B—H, B-halogen, B-alkyl, B—OH, or B—NH₂.

25. The compound of claim 23, wherein the compound is defined by Formula XIA, or a pharmaceutically acceptable salt thereof: 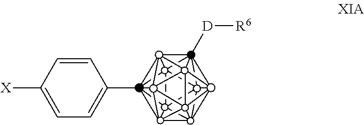
wherein
● is a carbon atom or a boron atom; and
◯ is C—H, C-halogen, C-alkyl, C—OH, C—NH₂, B—H, B-halogen, B-alkyl, B—OH, or B—NH₂
D is —S—, —S(O)—, —S(O)(O)—, —S(O)(NH)—, —P(O)(OH)O—, —P(O)(OH)NH—, or —O—;
X is OH, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, or substituted or unsubstituted C₄-C₂₀ alkylheterocycloalkyl; and
R² is H, OH, halogen, or substituted or unsubstituted C₁-C₄ alkyl.

26. The compound of claim 23, wherein X is OH.

27. The compound of claim 23, wherein D is —S—, or —S(O)(O)—.

28. The compound of claim 23, wherein R⁶ is substituted or unsubstituted C₁-C₂₀ alkyl.

29. The compound of claim 23, wherein R⁶ is C₁-C₂₀ alkyl optionally substituted with OH.

30. The compound of claim 23, wherein R⁶ is substituted or unsubstituted C₁-C₆ alkyl.

31. The compound of claim 23, wherein R⁶ is substituted or unsubstituted C₁-C₃ alkyl.

32. The compound of claim 23, wherein R⁶ is unsubstituted C₁-C₃ alkyl.

33. The compound of claim 23, wherein R⁶ is C₁-C₃ alkyl substituted with OH.

34. The compound of claim 23, wherein R⁶ is selected from the group consisting of:

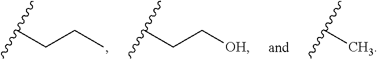

35. (canceled)

36. (canceled)

37. The compound of claim 23, wherein D-R⁶ is selected from the group consisting of:

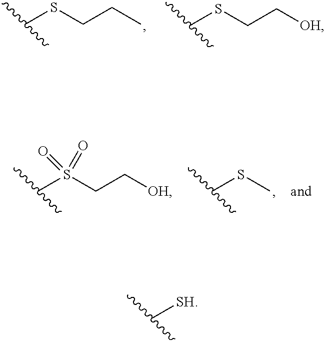

38. (canceled)

39. (canceled)

40. The compound of claim 23, wherein ● is a carbon atom and ◯ is B—H.

41. A compound comprising: 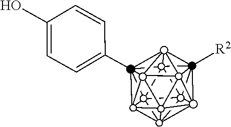
wherein
● is a carbon atom;
◯ is B—H; and
R² is selected from the group consisting of: 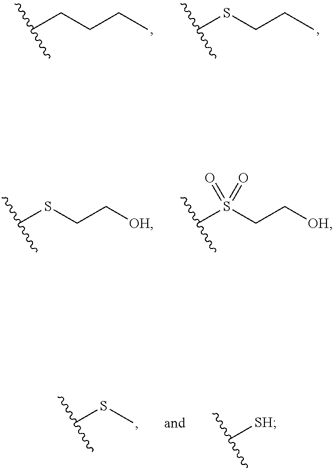
and pharmaceutically acceptable salts thereof.

42. The compound of claim 41, wherein the compound is selected from the group consisting of: 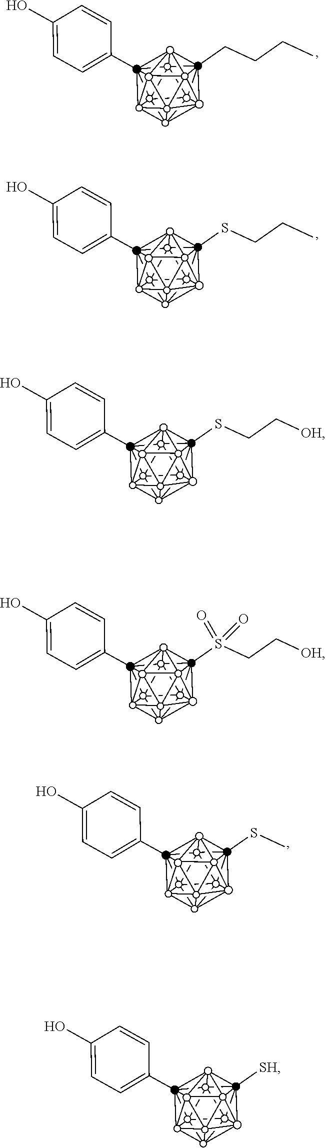
and pharmaceutically acceptable salts thereof, where ● is a carbon atom, and ◯ is B—H.

43. The compound of claim 41, wherein the compound is selected from the group consisting of: 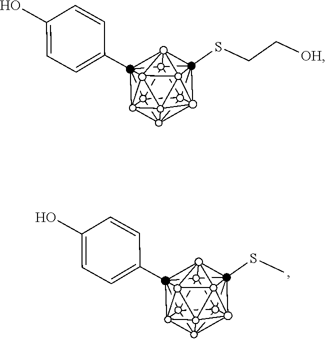
and pharmaceutically acceptable salts thereof, where ● is a carbon atom, and ◯ is B—H.

44. (canceled)

45. The compound of claim 23, wherein the compound is a selective ERβ agonist, wherein the compound is a selective ERβ agonist in both a human model and a mouse model, wherein the compound has an EC₅₀ of 200 nM or less at estrogen receptor beta (ERβ) in both a human model and a mouse model, wherein the compound has an ERβ-to-ERα agonist ratio of 8 or more in both a human model and a mouse model, or a combination thereof.

46.-55. (canceled)

56. A pharmaceutical composition comprising the compound of claim 23 and a pharmaceutically acceptable excipient.

57. A method of evaluating the clinical efficacy of an ERβ agonist in a human patient, the method comprising administering the ERβ agonist to a non-human preclinical species model; wherein the ERβ agonist has an ERβ-to-ERα ratio of 8 or more in both a human model and the non-human preclinical species model.

58.-73. (canceled)
